(19) United States
(12) Patent Application Publication
(10) Pub. No.: US 2012/0130144 A1
(43) Pub. Date: May 24, 2012

(54) TREATMENT OF LUNG CANCER WITH A NITROBENZAMIDE COMPOUND IN COMBINATION WITH A GROWTH FACTOR INHIBITOR

(75) Inventors: Barry M. Sherman, Hillsbourogh, CA (US); Charles Bradley, Half Moon Bay, CA (US); Valeria S. Ossovskaya, San Francisco, CA (US)

(73) Assignee: BiPar Sciences, Inc., South San Francisco, CA (US)

(21) Appl. No.: 13/146,865

(22) PCT Filed: Feb. 4, 2010
(86) PCT No.: PCT/US10/23137

§ 371 Date: Dec. 22, 2011

Related U.S. Application Data
(60) Provisional application No. 61/149,977, filed on Feb. 4, 2009.

(51) Int. Cl. A61N 5/00; A61K 39/395; A61K 39/00; A61K 31/519; A61K 31/42; A61K 31/4709; A61K 38/19; A61K 38/12; A61K 31/4985; A61K 31/4545; A61K 31/47; A61K 38/16; A61K 31/444; A61K 38/44; A61K 31/53; A61K 31/58; A61K 31/553; A61K 31/502; A61K 31/506; A61K 31/675; A61P 35/00; A61K 31/454; A61K 31/404; A61K 48/00; A61K 31/517

(52) U.S. Cl. 600/1; 514/252.16; 514/266.24; 424/136.1; 424/198.1; 514/267; 514/378; 424/138.1; 514/313; 514/266.22; 514/7.6; 514/249; 514/318; 514/312; 514/8.1; 514/332; 424/94.4; 514/243; 514/171; 514/211.08; 514/333; 514/248; 514/275; 424/142.1; 514/81; 424/133.1; 514/264.11; 514/323; 514/414; 514/418; 514/314; 514/44 R

(57) ABSTRACT

In one aspect, the present invention provides a method of treating lung cancer, comprising administering to a subject at least one PARP inhibitor in combination with at least one growth factor inhibitor. In another aspect, the present invention provides a method of treating non-small cell lung cancer comprising administering to a subject at least one PARP inhibitor in combination with at least one growth factor inhibitor.

### CROSS-REFERENCE TO RELATED APPLICATIONS

This application claims the benefit of U.S. Provisional Application No. 61/149,977 filed Feb. 4, 2009, the disclosure of which is incorporated herein by reference in its entirety.

### BACKGROUND OF THE INVENTION

Cancer is a group of diseases characterized by aberrant control of cell growth. The annual incidence of cancer is estimated to be in excess of 1.3 million in the United States alone. While surgery, radiation, chemotherapy, and hormones are used to treat cancer, it remains the second leading cause of death in the U.S. It is estimated that over 560,000 Americans will die from cancer each year.

Cancer cells simultaneously activate several pathways that positively and negatively regulate cell growth and cell death. This trait suggests that the modulation of cell death and survival signals could provide new strategies for improving the efficacy of current chemotherapeutic treatments.

Lung cancer is a disease of uncontrolled cell growth in tissues of the lung. This growth may lead to metastasis, which is invasion of adjacent tissue and infiltration beyond the lungs. The vast majority of primary lung cancers are carcinomas of the lung, derived from epithelial cells. Lung cancer, the most common cause of cancer-related death in men and the second most common in women, is responsible for 1.3 million deaths worldwide annually. The main types of lung cancer are small cell lung carcinoma and non-small cell lung carcinoma. This distinction is important, because the treatment varies; non-small cell lung carcinoma (NSCLC) is sometimes treated with surgery, while small cell lung carcinoma (SCLC) usually responds better to chemotherapy and radiation. The most common cause of lung cancer is long-term exposure to tobacco smoke. The occurrence of lung cancer in nonsmokers, who account for as many as 15% of cases, is often attributed to a combination of genetic factors, radon gas, asbestos, and air pollution, including secondhand smoke. Lung cancer may be seen on chest x-ray and computed tomography (CT scan). The diagnosis is confirmed with a biopsy. This is usually performed via bronchoscopy or CT-guided biopsy. Treatment and prognosis depend upon the histological type of cancer, the stage (degree of spread), and the patient's performance status. Possible treatments include surgery, chemotherapy, and radiotherapy. With treatment, the five-year survival rate is 14%.

Although there are limited therapeutic options for cancer treatment, variants of cancers, including lung cancer, are especially difficult to treat because they can be refractory to standard chemotherapeutic treatment. There is thus a need for an effective treatment for cancer in general, and cancer variants in particular, including lung cancer.

### BRIEF SUMMARY OF THE INVENTION

In one aspect, the present invention provides a method of treating lung cancer in a patient, comprising administering to the patient at least one PARP inhibitor in combination with at least one growth factor inhibitor. In another aspect, the present invention provides a method of treating non-small cell lung cancer (NSCLC) in a patient, comprising administering to the patient at least one PARP inhibitor in combination with at least one growth factor inhibitor. In yet another aspect, the present invention provides a method of treating lung cancer in a patient, comprising: testing a sample from the patient for PARP expression; and if the PARP expression exceeds a predetermined level, administering to the patient at least one PARP inhibitor and at least one growth factor inhibitor.

In practicing any of the subject methods disclosed herein, in some embodiments, at least one therapeutic effect is obtained, said at least one therapeutic effect being reduction in size of a lung tumor including a non-small cell lung tumor, reduction in metastasis, complete remission, partial remission, stable disease, or a pathologic complete response. In some embodiments, an improvement of clinical benefit rate (CBR=CR+PR+SD≧6 months) is obtained as compared to treatment with the growth factor inhibitor but without the PARP inhibitor. In some embodiments, the improvement of clinical benefit rate is at least about 60%. In some embodiments, the PARP inhibitor is 4-iodo-3-nitrobenzamide or a metabolite thereof. In some embodiments, the PARP inhibitor is of Formula (IIa) or a metabolite thereof:

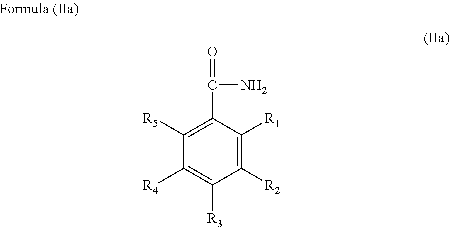

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, and R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄, and R₅ group that is a nitroso, hydroxyamino, hydroxy or amino group. In some embodiments, the compounds of (2) are such that the iodo the iodo group is always adjacent an R₁, R₂, R₃, R₄, and R₅ group that is a nitroso, hydroxyamino, or amino group.

In some embodiments, the growth factor is selected from the group consisting of epidermal growth factor (EGF), nerve growth factor (NGF), insulin-like growth factor I (IGF1), hepatocyte growth factor (HGF), vascular endothelial growth factor (VEGF), hepatoma-derived growth factor (HDGF), fibroblast growth factor (FGF), and platelet derived growth factor (PDGF). In some embodiments, the growth factor inhibitor is an epidermal growth factor receptor (EGFR) inhibitor. In some embodiments, the method further comprises surgery, radiation therapy, chemotherapy, gene therapy, DNA therapy, viral therapy, DNA therapy, adjuvant therapy, neoadjuvant therapy, RNA therapy, immunotherapy, nanotherapy or a combination thereof.

In some embodiments, the lung cancer is a metastatic lung cancer. In some embodiments, the lung cancer is at stage I, stage II, or stage III. In some embodiments, the lung cancer is a non-small cell lung carcinoma (NSCLC). In some embodiments, the non-small cell lung carcinoma is a squamous cell carcinoma, adenocarcinoma, or large cell carcinoma. In some embodiments, the lung cancer is a small cell lung carcinoma (SCLC). In some embodiments, the lung cancer is deficient in homologous recombination DNA repair.

In some embodiments, the growth factor inhibitor is administered as a parenteral injection or infusion. In some embodiments, the PARP inhibitor is 4-iodo-3-nitrobenzamide, which is administered orally, or as a parenteral injection or infusion, or by inhalation. In some embodiments, the method further comprises administering to the patient one or more of the group consisting of a cyclodextrin, a surfactant, and a co-solvent. In some embodiments, the cyclodextrin comprises one or more of hydroxypropyl-β-cyclodextrin, hydroxypropyl-γ-cyclodextrin, and sulfobutyl ether-β-cyclodextrin.

In a further aspect is provided pharmaceutical compositions of at least one PARP inhibitor and at least one growth factor inhibitor, such as those described herein, for the preparation of a medicament for use in the treatment of lung cancer. The compositions and formulations described herein may be used in the preparation of medicaments suitable for use in the methods such as described herein and for the treatment of lung cancer, including the sub-types of lung cancer described herein (e.g., small cell lung cancer, non-small cell lung cancer, etc.). In some embodiments, the PARP inhibitor is 4-iodo-3-nitrobenzamide, or a pharmaceutically acceptable salt, isomer, solvate or tautomer thereof. In some embodiments are provided a pharmaceutical composition comprising at least one PARP inhibitor in combination with at least one growth factor inhibitor, wherein said PARP inhibitor is of Formula (Ia), or a metabolite thereof:

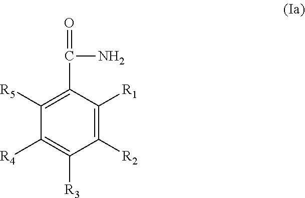

wherein R₁, R₂, R₃, R₄, and R₅ are, independently selected from the group consisting of hydrogen, hydroxy, amino, nitro, nitroso, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents is always nitro, and at least one substituent positioned adjacent to a nitro is always iodo, or a pharmaceutically acceptable salt, solvate, isomer, or tautomer thereof, and, wherein the growth factor inhibitor is selected from the group consisting of AEE788, GW-974, BIBW 2992, catumaxomab, EGF vaccine, icotinib, leflunomide, necitumumab, neratinib, pertuzumab, PF-299804, zalutumumab, CNTF, tanezumab, dalotuzumab, AMG-479, rilotumumab, lanreotide, OSI 906, pasireotide, PF-2341066, MetMab, XL-184, aflibercept, apatinib, BIBF-1120, PAM-1, XL-999, brivanib, fluocinolone, midostaurin, motesanib, OTS-102, OSI-632, vatalanib, pazopanib, BMS-690514, ramucirumab, ridoforolimus, tivozanib, alacizumab pegol, PD173074, PHA 665752, DMQ, SU4312, K252a, XL-647, VEGF-Trap-Eye, pirfenidone, masitinib, and nilotinib. In some embodiments, the PARP inhibitor is 4-iodo-3-nitrobenzamide, or a pharmaceutically acceptable salt thereof.

In another aspect is provided the use of the pharmaceutical compositions described herein for the manufacture of a medicament for treating lung cancer. For example, use as provided herein with respect to the methods described herein.

The present invention also relates to a kit comprising at least one PARP inhibitor and at least one growth factor inhibitor, such as those described herein and a solubilizer, where the solubilizer is a cyclodextrin, a surfactant, a co-solvent, or a mixture of (1) a cylodextrin and a surfactant, (2) a cyclodextrin and a co-solvent, (3) a surfactant and a co-solvent, or (4) a cyclodextrin, a surfactant, and a co-solvent, and packaging. The kits in some rembodiments include the pharmaceuctical formulations described herein.

The present invention also relates more specifically to a kit comprising a PARP inhibitor compound that is 4-iodo-3-nitrobenzamide or a salt, solvate, isomer, or thereof, at least one growth factor inhibitor and a solubilizer, where the solubilizer is a cyclodextrin, a surfactant, a co-solvent, or a mixture of (1) a cylodextrin and a surfactant, (2) a cyclodextrin and a co-solvent, (3) a surfactant and a co-solvent, or (4) a cyclodextrin, a surfactant, and a co-solvent, and packaging.

In the kits provided herein, in some embodiments, the formulation is an oral formulation, such as a tablet or capsule. In some embodiments, the formulation is a parenteral formulation, such as in intravenous or intraperitoneal injection.

In another aspect is provided the use of at least one PARP inhibitor and at least one growth factor inhibitor (or a composition including at least one PARP inhibitor and at least one growth factor inhibitor), such as those described herein, for use in the treatment of lung cancer, including the sub-types of lung cancer described herein and according to the methods described herein for such treatment.

In some embodiments are provided methods of treating lung cancer in a patient, comprising administering to the patient having lung cancer at least one PARP inhibitor in combination with at least one growth factor inhibitor, wherein said PARP inhibitor is of Formula (Ia), or a metabolite thereof:

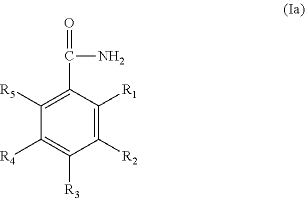

wherein R₁, R₂, R₃, R₄, and R₅ are, independently selected from the group consisting of hydrogen, hydroxy, amino, nitro, nitroso, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents is always nitro, and at least one substituent positioned adjacent to a nitro is always iodo, or a pharmaceutically acceptable salt, solvate, isomer, or tautomer thereof, and, wherein the growth factor inhibitor is selected from the group consisting of AEE788, GW-974, BIBW 2992, catumaxomab, EGF vaccine, icotinib, leflunomide, necitumumab, neratinib, pertuzumab, PF-299804, zalutumumab, CNTF, tanezumab, dalotuzumab, AMG-479, rilotumumab, lanreotide, OSI 906, pasireotide, PF-2341066, MetMab, XL-184, aflibercept, apatinib, BIBF-1120, PAM-1, XL-999, brivanib, fluocinolone, midostaurin, motesanib, OTS-102, OSI-632, vatalanib, pazopanib, BMS-690514, ramucirumab, ridoforolimus, tivozanib, alacizumab pegol, PD173074, PHA 665752, DMQ, SU4312, K252a, XL-647, VEGF-Trap-Eye, pirfenidone, masitinib, and nilotinib. In some embodiments, at least one therapeutic effect is obtained, said at least one therapeutic effect being reduction in size of a lung tumor, reduction in metastasis, complete remission, partial remission, stable disease, or a pathologic complete response. In some embodiments, wherein an improvement of clinical benefit rate (CBR=CR (complete remission)+PR (partial remission)+SD (stable disease)≧6 months) is obtained as compared to treatment with the growth factor inhibitor administered without the PARP inhibitor. In some embodiments, the improvement of clinical benefit rate is about 60% or higher. In some embodiments the PARP inhibitor is 4-iodo-3-nitrobenzamide, or pharmaceutically acceptable salt thereof. In some embodiments, the growth factor is an epidermal growth factor receptor (EGFR) inhibitor such as BIBW 2992, catumaxomab, XL-647, EGF vaccine (CIMAB/Micromet/Biocon/Bioven), icotinib, leflunomide, necitumumab, neratinib, GW-974, PF-299804, or zalutumumab. In some embodiments, the growth factor inhibitor is a nerve growth factor receptor (NGFR) inhibitor such as CNTF, K252a, or tanezumab. In some embodiments, the growth factor inhibitor is an insulin-like growth factor I (IGF1) receptor inhibitor such as dalotuzumab, AMG-479, rilotumumab, lanreotide, OSI 906, or pasireotide. In some embodiments, the growth factor inhibitor is a hepatocyte growth factor receptor (HGFR) inhibitor such as PF-2341066, MetMab, PHA 665752, or XL-184. In some embodiments, the growth factor inhibitor is a vascular endothelial growth factor receptor (VEGFR) inhibitor such as aflibercept, apatinib, BIBF-1120, brivani, fluocinolone, midostaurin, motesanib, OTS-102, OSI-632, vatalanib, pazopanib, BMS-690514, ramucirumab, ridoforolimus, tivozanib, XL-647, VEGF-Trap-Eye, alacizumab pegol, SU4312, or XL-184. In some embodiments, the growth factor inhibitor is a fibroblast growth factor receptor (FGFR) inhibitor such as BIBF-1120, brivanib, PAM-1, pirfenidone, PD 173074, or masitib. In some embodiments, the growth factor inhibitor is a platelet derived growth factor receptor (PDGFR) inhibitor such as BIBF-1120, leflunomide, masitinib, motesanib, nilotinib, pazopanib, pirfenidone, DMPQ, SU4312, or tivozanib. In some embodiments, the growth factor inhibitor is a platelet derived growth factor receptor (PDGFR) inhibitor and is pazopanib. In some embodiments, the growth factor inhibitor is AEE788. In some embodiments, the method further comprises surgery, radiation therapy, chemotherapy, gene therapy, DNA therapy, viral therapy, DNA therapy, adjuvant therapy, neoadjuvant therapy, RNA therapy, immunotherapy, nanotherapy or a combination thereof. In some embodiments, the lung cancer is a metastatic lung cancer. In some embodiments, the lung cancer is at stage I, stage II, or stage III. In some embodiments, the lung cancer is a non-small cell lung carcinoma (NSCLC). In some embodiments, the non-small cell lung carcinoma is a squamous cell carcinoma, adenocarcinoma, or large cell carcinoma. In some embodiments, the lung cancer is a small cell lung carcinoma (SCLC). In some embodiments, the lung cancer is deficient in homologous recombination DNA repair. In some embodiments, the growth factor inhibitor is administered as a parenteral injection or infusion. In some embodiments, wherein the PARP inhibitor is 4-iodo-3-nitrobenzamide, which is administered orally, or as a parenteral injection or infusion, or inhalation. In some embodiments, one or more of the group consisting of a cyclodextrin, a surfactant, and a co-solvent is administered in combination with the PARP inhibitor. In some embodiments, the cyclodextrin is selected from the group consisting of hydroxypropyl-β-cyclodextrin, hydroxypropyl-γ-cyclodextrin, and sulfobutyl ether-β-cyclodextrin, or a combination thereof.

In some embodiments, the lung cancer is a non-small cell lung carcinoma (NSCLC). In some embodiments, at least one therapeutic effect is obtained, and said at least one therapeutic effect is reduction in size of a non-small cell lung tumor, reduction in metastasis, complete remission, partial remission, stable disease, or a pathologic complete response. In some embodiments, the improvement of clinical benefit rate (CBR=CR (complete remission)+PR (partial remission)+SD (stable disease)≧6 months) is obtained as compared to treatment with the growth factor inhibitor administered without the PARP inhibitor. In some embodiments, the improvement of clinical benefit rate is about 60% or higher. In some embodiments, the PARP inhibitor is 4-iodo-3-nitrobenzamide, or a pharmaceutical salt thereof, or a metabolite thereof. In some embodiments, the growth factor is an epidermal growth factor receptor (EGFR) inhibitor such as BIBW 2992, catumaxomab, EGF vaccine (CIMAB/Micromet/Biocon/Bioven), icotinib, leflunomide, necitumumab, neratinib, or zalutumumab. In some embodiments, the growth factor inhibitor is pazopanib. In some embodiments, the growth factor inhibitor is AEE788. In some embodiments, the method further includes surgery, radiation therapy, chemotherapy, gene therapy, DNA therapy, viral therapy, DNA therapy, adjuvant therapy, neoadjuvant therapy, RNA therapy, immunotherapy, nanotherapy or a combination thereof. In some embodiments, the non-small cell lung cancer is a metastatic non-small cell lung cancer. In some embodiments, the non-small cell lung carcinoma is a squamous cell carcinoma, adenocarcinoma, or large cell carcinoma. In some embodiments, the non-small cell lung cancer is deficient in homologous recombination DNA repair. In some embodiments, the growth factor inhibitor is administered as a parenteral injection or infusion. In some embodiments, the PARP inhibitor is PARP inhibitor is 4-iodo-3-nitrobenzamide, or pharmaceutically acceptable salt thereof. In some embodiments, the 4-iodo-3-nitrobenzamide, or pharmaceutically acceptable salt thereof, is administered orally, or as a parenteral injection or infusion, or by inhalation.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows that BA potentiates the activity of gefitinib (IRESSA), an EGFR inhibitor, in the HCC827 cell line.

FIG. 2a-2c illustrate that BA potentiates antiproliferative effect of growth factor inhibitors; where FIG. 2a-b illustrate the effect of BA, gefitinib and combination of BA with gefitinib on proliferation of lung carcinoma HCC827 cells; FIG. 2c reports FACS-based cell cycle analysis showing that BA induced number of dead cells (sub-G1) and reduced number of cells in G1 and S phases of cell cycle in gefitinib-treated HCC827 cells. Data for BNO are shown in FIG. 2d-2f.

FIG. 3 shows data from the CellTiter 96® Aqueous Cell Proliferation Assay for gefitinib (EGFR inhibitor) with and without BA for the HCC827 cell line; absorbance at 490 nm vs. gefitinib concentration are plotted for indicated concentrations of BA and gefitinib alone.

FIG. 4 shows data from the CellTiter 96® Aqueous Cell Proliferation Assay for PD 173074 (FGFR inhibitor), with and without BA for the HCC827 cell line; absorbance at 490 nm vs. PD 173074 concentration are plotted for indicated concentrations of BA and PD 173074 alone.

FIG. 5 shows data from the CellTiter 96® Aqueous Cell Proliferation Assay for picropodophyllotoxin (PPP) (IGF1R inhibitor, a IGF receptor subtype), with and without BA for the HCC827 cell line; absorbance at 490 nm vs. PPP concentration are plotted for indicated concentrations of BA and PPP alone.

FIG. 6 shows data from the CellTiter 96® Aqueous Cell Proliferation Assay for PHA 665752 (HGFR inhibitor), with and without BA for the HCC827 cell line; absorbance at 490 nm vs. PHA 665752 concentration are plotted for indicated concentrations of BA and PHA 665752 alone.

FIG. 7 shows data from the CellTiter 96® Aqueous Cell Proliferation Assay for DMPQ dihydrochloride (PDGFR inhibitor (specifically PDGFR-beta)), with and without BA for the HCC827 cell line; absorbance at 490 nm vs. DMPQ dihydrochloride concentration are plotted for indicated concentrations of BA and DMPQ dihydrochloride alone.

FIG. 8 shows data from the CellTiter 96® Aqueous Cell Proliferation Assay for SU4312 (selective inhibitor of VEGFR and PDGFR), with and without BA for the HCC827 cell line; absorbance at 490 nm vs. SU4312 concentration are plotted for indicated concentrations of BA and SU4312 alone.

FIG. 9 shows data from the CellTiter 96® Aqueous Cell Proliferation Assay for K252a (NGFR inhibitor), with and without BA for the HCC827 cell line; absorbance at 490 nm vs. K252a concentration are plotted for indicated concentrations of BA and K252a alone.

### DETAILED DESCRIPTION OF THE INVENTION

In some embodiments, the present invention provides a method of treating cancer in a patient, comprising administering to the patient at least one PARP inhibitor in combination with at least one growth factor inhibitor. In some embodiments, at least one therapeutic effect is obtained, said at least one therapeutic effect being reduction in size of a tumor, reduction in metastasis, complete remission, partial remission, pathologic complete response, or stable disease. In some embodiments, a comparable clinical benefit rate (CBR=CR+PR+SD≧6 months) is obtained with treatment of the PARP inhibitor as compared to treatment with a growth factor inhibitor. In some embodiments, the improvement of clinical benefit rate is at least about 60%. In some embodiments, the PARP inhibitor is a PARP-1 inhibitor. In some embodiments, the PARP inhibitor is of Formula (IIa) or a metabolite thereof:

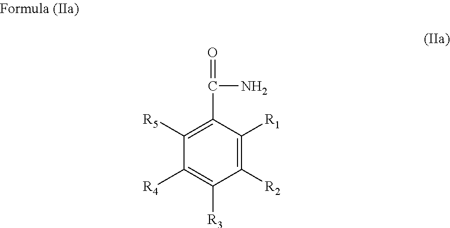

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, and R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄, and R₅ group that is a nitroso, hydroxyamino, hydroxy or amino group. In some embodiments, the compounds of (2) are such that the iodo the iodo group is always adjacent a R₁, R₂, R₃, R₄, and R₅ group that is a nitroso, hydroxyamino, or amino group. In some embodiments, the PARP 1 inhibitor is 4-iodo-3-nitrobenzamide or a metabolite thereof.

In some embodiments, the treatment comprises a treatment cycle of at least 11 days, wherein on days 1, 4, 8 and 11 of the cycle, the patient receives about 1 to about 100 mg/kg of 4-iodo-3-nitrobenzamide or a molar equivalent of a metabolite thereof. In some embodiments, 4-iodo-3-nitrobenzamide is administered orally, as a parenteral injection or infusion, or inhalation. In some embodiments, the treatment cycle is about 11 to about 30 days in length.

In some embodiments, the growth factor inhibitor is an inhibitor of epidermal growth factor (EGF), nerve growth factor (NGF), insulin-like growth factor I (IGF1), hepatocyte growth factor (HGF), vascular endothelial growth factor (VEGF), hepatoma-derived growth factor (HDGF), fibroblast growth factor (FGF), platelet derived growth factor (PDGF), or a combination thereof. In some embodiments, the growth factor inhibitor is EGFR inhibitor. In some embodiments, the method further comprises administering to the patient a PARP inhibitor in combination with more than one growth factor inhibitors. The growth factor inhibitor is administered prior to, concomitant with or subsequent to administering the PARP inhibitor. In some embodiments, the method further comprises surgery, radiation therapy, chemotherapy, gene therapy, DNA therapy, adjuvant therapy, neoadjuvant therapy, RNA therapy, DNA therapy, viral therapy, immunotherapy, nanotherapy or a combination thereof.

In some embodiments, the cancer is an adrenal cortical cancer, anal cancer, aplastic anemia, bile duct cancer, bladder cancer, bone cancer, bone metastasis, CNS tumors, peripheral CNS cancer, Castleman's Disease, cervical cancer, childhood Non-Hodgkin's lymphoma, colon and rectum cancer, esophagus cancer, Ewing's family of tumors, eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, vaginal cancer, vulvar cancer, Waldenstrom's macroglobulinemia or cancers of viral origin. In some embodiments, the cancer is lung cancer. In some embodiments, the lung cancer is a metastatic lung cancer. In some embodiments, the lung cancer is at stage I, II or III. In some embodiments, the lung cancer is a non-small cell lung carcinoma (NSCLC). In some embodiments, the NSCLC is a squamous cell carcinoma, an adenocarcinoma, or a large cell carcinoma. In some embodiments, the lung cancer is a small cell lung carcinoma (SCLC). In some embodiments, the lung cancer is deficient in homologous recombination DNA repair.

Some embodiments described herein provide a method of treating lung cancer in a patient, comprising administering to the patient at least one PARP inhibitor and at least one growth factor inhibitor. In some embodiments, at least one therapeutic effect is obtained, said at least one therapeutic effect being reduction in size of a lung tumor, reduction in metastasis, complete remission, partial remission, pathologic complete response, or stable disease. In some embodiments, a comparable clinical benefit rate (CBR=CR+PR+SD≧6 months) is obtained with treatment of the PARP inhibitor as compared to treatment with a growth factor inhibitor. In some embodiments, the improvement of clinical benefit rate is at least about 60%. In some embodiments, the PARP inhibitor is a PARP-1 inhibitor. In some embodiments, the PARP inhibitor is of Formula (IIa) or a metabolite thereof:

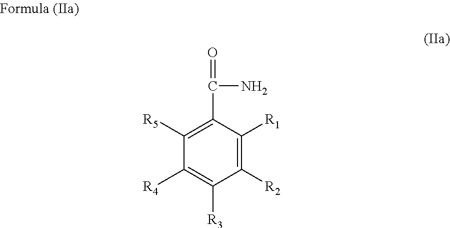

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, and R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄, and R₅ group that is a nitroso, hydroxyamino, hydroxy or amino group. In some embodiments, the compounds of (2) are such that the iodo the iodo group is always adjacent a R₁, R₂, R₃, R₄, and R₅ group that is a nitroso, hydroxyamino, or amino group. In some embodiments, the PARP 1 inhibitor is 4-iodo-3-nitrobenzamide or a metabolite thereof.

Some embodiments described herein provide a method of treating non-small cell lung cancer (NSCLC) in a patient, comprising administering to the patient at least one PARP inhibitor and at least one growth factor inhibitor. In some embodiments, at least one therapeutic effect is obtained, said at least one therapeutic effect being reduction in size of a non-small cell lung tumor, reduction in metastasis, complete remission, partial remission, pathologic complete response, or stable disease. In some embodiments, a comparable clinical benefit rate (CBR=CR+PR+SD≧6 months) is obtained with treatment of the PARP inhibitor as compared to treatment with a growth factor inhibitor. In some embodiments, the improvement of clinical benefit rate is at least about 60%. In some embodiments, the PARP inhibitor is a PARP-1 inhibitor. In some embodiments, the PARP inhibitor is of Formula (IIa) or a metabolite thereof:

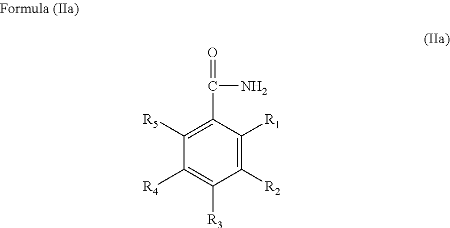

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, and R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄, and R₅ group that is a nitroso, hydroxyamino, hydroxy or amino group. In some embodiments, the compounds of (2) are such that the iodo the iodo group is always adjacent a R₁, R₂, R₃, R₄, and R₅ group that is a nitroso, hydroxyamino, or amino group. In some embodiments, the PARP 1 inhibitor is 4-iodo-3-nitrobenzamide or a metabolite thereof.

In some embodiments, the treatment comprises a treatment cycle of at least 11 days, wherein on days 1, 4, 8 and 11 of the cycle, the patient receives about 1 to about 100 mg/kg of 4-iodo-3-nitrobenzamide or a molar equivalent of a metabolite thereof. In some embodiments, 4-iodo-3-nitrobenzamide is administered orally, as a parenteral injection or infusion, or inhalation. In some embodiments, the treatment cycle is about 11 to about 30 days in length.

In some embodiments, the growth factor inhibitor is an inhibitor of epidermal growth factor (EGF), nerve growth factor (NGF), insulin-like growth factor I (IGF1), hepatocyte growth factor (HGF), vascular endothelial growth factor (VEGF), hepatoma-derived growth factor (HDGF), fibroblast growth factor (FGF), platelet derived growth factor (PDGF), or a combination thereof. In some embodiments, the growth factor inhibitor is EGFR inhibitor. In some embodiments, the growth factor inhibitor is an inhibitor of epidermal growth factor (EGF), nerve growth factor (NGF), insulin-like growth factor I (IGF1), hepatocyte growth factor (HGF), vascular endothelial growth factor (VEGF), hepatoma-derived growth factor (HDGF), fibroblast growth factor (FGF), or platelet derived growth factor (PDGF) as described herein. In some embodiments, the growth factor inhibitor is EGFR inhibitor. In some embodiments, the method further comprises administering to the patient a PARP inhibitor in combination with more than one growth factor inhibitors. The growth factor inhibitor is administered prior to, concomitant with or subsequent to administering the PARP inhibitor. In some embodiments, the method further comprises surgery, radiation therapy, chemotherapy, gene therapy, DNA therapy, adjuvant therapy, neoadjuvant therapy, RNA therapy, DNA therapy, viral therapy, immunotherapy, nanotherapy or a combination thereof.

In some embodiments, the growth factor inhibitor is an inhibitor of epidermal growth factor (EGF). Exemplary EGF inhibitors include, but are not limited to: BIBW 2992 (also known as: BIBW2992, TOVOK; Boehringer Ingelheim), MDX-447 (Medarex), catumaxomab (also known as: Removal, triomab-1; Trion Pharma), cetuximab (also known as: Anti-EGFR monoclonal antibody 225, C 225, ERBITUX, IMC-C225; Bristol-Myers Squibb Co.), EGF vaccine (CIMAB/Micromet/Biocon/Bioven), erlotinib (also known as: CP 358774, NSC 718781, OSI 774, R1415, RG1415, TARCEVA; Chugai Pharmaceutical, Genentech Inc.), gefitinib (also known as: IRESSA, ZD-1839, IRESSAT, M-387783, M-537194, M-523595; AstraZeneca plc), icotinib (also known as: BPI-1096, BPI-2009H; Zhejiang Beta Pharma), lapatinib (also known as: 572016, GW2016, GW572016, GW572016F, TYCERB, TYKERB, TYVERB; GlaxoSmithKline), lapatinib+pazopanib (also known as: TYKERB+ARMALA; GlaxoSmithKline), XL-647, matuzumab (also known as EMD-7200), leflunomide (also known as: ARAVA, HWA 486, SU 101; sanofi-aventis), necitumumab (also known as: IMC 11F8, IMC-11F8; ImClone Systems), neratinib (also known as: HKI-272; Pfizer, Inc), nimotuzumab (also known as: Anti-EGFR mAb hR3, BIOMAB EGFR, h-R3, hR3, OSAG101, TheraCIM, TheraCIM hR3, THERALOC, VECTHIX; Biocon Biopharmaceuticals), panitumumab (also known as: ABX-EGF, E7.6.3, rHuMAb-EGFr, VECTIBIX; Amgen/Takeda), pertuzumab (also known as: 2C4 antibody (Genentech), Omnitarg, R-1273, R1273, RG-1273, RG1273, rhuMAb 2C4), Polyphenon E Ointment (also known as: sinecatechins, VEREGEN; Epitome Pharmaceuticals), trastuzumab (also known as: anti-HER-2 MAb, Genentech, anti-HER-2 MAb, Roche, HER-2 MAb, Genentech, HER-2 MAb, Roche, HERCEPTIN, R-597, R597, RG-597, RG597, rhuMAb HER2, Ro-45-2317), trastuzumab-DM1 (also known as: HERCEPTIN+DM1, Pro-132365, R-3502, R3502, RG-3502, RG3502, T-DM1, trastuzumab-Mcc-DM1; Hoffmann-La Roche), vandetanib (also known as: AZD6474, ZACTIMA, ZD6474, AstraZeneca plc), pelitinib (also know as: EKB-569) and zalutumumab (also known as: HUMAX-EGFR; Genmab).

In some embodiments, the growth factor inhibitor is an inhibitor of nerve growth factor (NGF). Exemplary NGF inhibitors include, but are not limited to: CNTF (also known as: NTC-201E, NTC-501; Neurotech), K25a (LC Labs), and tanezumab (also known as: PF 4383119, PF-04383119, PF-4383119, R 624, RN624, RN624).

In some embodiments, the growth factor inhibitor is an inhibitor of insulin-like growth factor I (IGF1). Exemplary IGF inhibitors include, but are not limited to: dalotuzumab (also known as: F-50035, MK-0646, h7C10, A2CHM; Pierre Fabre S A), picropodophyllotoxin (also known as: Picropodophyllin, PPP, PPT, (5R,5aS,8aR,9R)-5,8,8a,9-Tetrahydro-9-hydroxy-5-(3,4,5-trimethoxyphenyl)-furo[3′,′: 6,7]naphtho[2,3-d]-1,3-dio xol-6(5aH)-one; Tocris Bioscience), figitumumab (also known as: CP 751871, CP-751, 871; Pfizer, Inc.), lanreotide (also known as: dermopeptin, somatuline, BIM-23014C, BN-52030, ipstyl, ITM-014, DC13-116, Angiopeptin; Ipsen, Inc.), OSI 906 (OSI Pharmaceuticals), AMG-479, and pasireotide (also known as: SOM 230, SOM230C (Novartis, Inc.).

In some embodiments, the growth factor inhibitor is an inhibitor of hepatocyte growth factor (HGF). Exemplary HGF inhibitors include, but are not limited to: PF-2341066 (also known as: PF-02341066; Pfizer, Inc.), MetMab (Genentech), PHA 665752 (Torcris Bioscience), and XL-184 (also known as: BMS-907351; Exelixis Inc/Bristol-Myers Squibb Co.).

In some embodiments, the growth factor inhibitor is an inhibitor of vascular endothelial growth factor (VEGF). Exemplary VEGF inhibitors include, but are not limited to: aflibercept (also known as: AVE 0005, AVE 005, AVE0005; Bayer Healthcare/Sanofi-Aventis), apatinib (also known as: YN-968D1, YN968D1; Advenchen, Inc.), axitinib (also known as: AG-13736, AG-013736, Agouron/Pfizer), bevacizumab (also known as: AVASTIN, R 435, R435, RG435; Genentech), BIBF-1120 (also known as: Vargatef, Boehringer Ingelheim), brivanib (also known as: BMS-582664, BMS-540215, IDDBCP180722; Bristol-Myers Squibb Co), semaxinib (also known as SU5416), XL-999 (Exelixis), cediranib (also known as: RECENTIN, AZD-2171; AstraZeneca plc), fluocinolone (also known as: MEDIDUR; ILUVIEN; Alimera Sciences Inc.), lapatinib, lapatinib+pazopanib (also known as: TYKERB+ARMALA, GlaxoSmithKline), linifanib (also known as: ABT-869, HT-1080, RG-3635, RG3635; Hoffmann-La Roche), midostaurin (also known as: 4-N benzoylstaurosporine, 4-N-benzoyl staurosporine, Benzoylstaurosporine, CGP 41251, N-benzoyl-staurosporine, PKC412, PKC412A; Novartis), motesanib (also known as: AMG-706; Amgen, Inc.), OTS-102 (OncoTherapy Science, Inc.), AE-941 (also known as: Neovastat; Aeterna Laboratories), pazopanib (also known as: GW-786034, VOTRIENT, ARMALA, 786034, GW-786034B; GlaxoSmithKline), alacizumab pegol, XL-647, BMS-690514, pegaptanib (also known as: Macuverse (Macugen), EYE-001 (OcuPhor), (OSI; Eyetech/IOMED) NX-1838), ramucirumab (also known as: IMC-2C6, IMC-1121, IMC-1121B; ImClone Systems Inc.), ranibizumab (also known as: Y0317, LUCENTIS, RG-3645; Genentech, Inc., Novartis, Inc), ridoforolimus (also known as: AP-23573, AP-573, Ariad573; deforolimus, MK-8669; Ariad/Merck & Co), sorafenib (also known as: BAY-43-9006; IDDBCP150446, NEXAVAR, BAY-54-9085, Bayer AG, Onyx Pharmaceuticals, Inc.), sunitinib (also known as: sutene, PHA-290940AD, SU-010398, SU-011248, SU-11248J, SU-12662, SUTENT, SU-11248; SUGEN Inc./Pfizer Inc., Pharmacia Corp.), tivozanib (also known as: KRN-951, AV-951, AVEO Pharmaceuticals Inc), vandetanib (also known as: AZD6474, ZACTIMA, ZD6474; AstraZeneca plc), VEGF-Trap-Eye (Bayer), SU4312 (Tocris Bioscience) and XL-184 (also known as: BMS-907351, Bristol-Myers Squibb Co/Exelixus, Inc.).

In some embodiments, the growth factor inhibitor is an inhibitor of hepatoma-derived growth factor (HDGF)

In some embodiments, the growth factor inhibitor is an inhibitor of fibroblast growth factor (FGF). Exemplary FGF inhibitors include, but are not limited to: BIBF-1120 (also known as: Vargatef, Boehringer Ingelheim), PAM-1, brivanib (also known as: BMS-582664, BMS-540215, IDDBCP180722; Bristol-Myers Squibb Co), XL-999 (Exelixis), pirfenidone (also known as: AMR-69, Deskar, S-7701, PIRESPA; Marnac Inc.), PD 173074 (also known as: STEMOLECULE; Tocris Bioscience), and masitinib (also known as AB-1010; AB Science). In some embodiments, the FGF inhibitor is an inhibitor selective for one or both of FGFR1 or FGFR3, e.g., PD 173074.

In some embodiments, the growth factor inhibitor is an inhibitor of platelet derived growth factor (PDGF). Exemplary PDGF inhibitors include, but are not limited to: axitinib (also known as: AG-13736, AG-013736, Agouron/Pfizer), XL-999 (Exelixis), BIBF-1120 (Boehringer Ingelheim), dasatinib (also known as: BMS-354825, SPRYCEL, SPRYCELL, Src/ABL, Bristol-Myers Squibb), leflunomide (also known as: A-77-1726 prodrug, Airohua, Arava, HWA-486, teriflunomide prodrug; sanofi-aventis), linifanib (also known as: ABT-869, HT-1080, RG-3635, RG3635; Abbott), masitinib (also known as: AB-1010, AB1010, AB Science), motesanib (also known as: AMG-706; Amgen, Inc.), nilotinib (also known as: AMN-107, TASIGNA; Novartis AG), pazopanib (also known as: 786034, ARMALA, GW-2286, GW-786034; GlaxoSmithKline), pirfenidone (also known as: AMR-69, Deskar, S-7701, PIRESPA; Marnac Inc.), sorafenib (also known as: Bay 43-9006, BAY 54-9085, Bay-43-9006, NEXAVAR, Onyx Pharmaceuticals), sunitinib (also known as: PNU 290940, PNU-290940, SU 011248, SU 11248, SU 11428, SUTENT; Pfizer, Inc.), DMPQ (e.g., DMPQ dihydrochloride; Torcris Bioscience), SU4312, and tivozanib (also known as: AV-951, KRN-951, KRN951; AVEO, Inc.). In some embodiments, the PDGF inhibitor is BIBF-1120, leflunomide, masitinib, motesanib, nilotinib, pazopanib, pirfenidone, DMPQ, SU4312, or tivozanib. In some embodiments, the PDGF inhibitor is an inhibitor selective for human vascular β-type platelet derived growth factor receptor tyrosine kinase (β-type PDGFR tyrosine kinase), e.g., DMPQ. In some embodiments, the PDGFR inhibitor is also selective for VEGFR (e.g., SU4312).

In some embodiments, the growth factor inhibitor is pelitinib, GW-974, tozasertib, MDX-447, antagonist D, ICRF, AE-941, OSI-632, NSTPBP-01250, PAM-1, XL-999, muparfostat, kahalalide F, vatalanib, squalamine, BMS-690514, PF-299804, AMG-479, elisidepsin, danusertib, rilotumumab, linifanib, XL-647, MetMAb, cixutumumab, ARQ-197, alacizumab pegol, OSI-906, pertuzumab, fenretinide, cediranib, axitinib, BIBW-2992, ramucirumab, vandetanib, PF-2341066, tivozanib, BIBF-1120, XL-184, BPI-2009-H, MK-0646, motesanib, figitumumab, necitumumab, neratinib, pazopanib, nimotuzumab, gefitinib, sorafenib, trastuzumab, CIMAB, dasatinib, cetuximab, panitumumab, sunitinib, erlotinib, or lapatinib. In some embodiments, the growth factor inhibitor is pelitinib, GW-974, tozasertib, MDX-447, antagonist D, ICRF, OSI-632, NSTPBP-01250, PAM-1, XL-999, muparfostat, kahalalide F, vatalanib, squalamine, BMS-690514, PF-299804, AMG-479, elisidepsin, danusertib, rilotumumab, XL-647, MetMAb, ARQ-197, alacizumab pegol, OSI-906, pertuzumab, BIBW-2992, ramucirumab, vandetanib, PF-2341066, tivozanib, BIBF-1120, XL-184, BPI-2009-H, MK-0646, motesanib, figitumumab, necitumumab, neratinib, pazopanib, or CIMAB. In some embodiments, the growth factor inhibitor is pelitinib, GW-974, MDX-447, ICRF, OSI-632, PAM-1, XL-999, vatalanib, BMS-690514, PF-299804, AMG-479, rilotumumab, XL-647, MetMAb, cixutumumab, alacizumab pegol, OSI-906, pertuzumab, BIBW-2992, ramucirumab, vandetanib, PF-2341066, tivozanib, BIBF-1120, XL-184, BPI-2009-H, MK-0646, motesanib, necitumumab, neratinib, pazopanib, or CIMAB. In some embodiments, the growth factor inhibitor is pazopanib.

In some embodiments, the growth factor inhibitor is AEE788, CP-751871, BIBW 2992, catumaxomab, cetuximab, EGF vaccine (CIMAB/Micromet/Biocon/Bioven), erlotinib, gefitinib, icotinib, lapatinib, lapatinib+pazopanib, leflunomide, necitumumab, neratinib, nimotuzumab, panitumumab, pertuzumab, Polyphenon E, trastuzumab, vandetanib, BMS-690514, zalutumumab, CNTF, tanezumab, dalotuzumab, AMG-479, figitumumab, rilotumumab, lanreotide, OSI 906, pasireotide, PF-2341066, MetMab, XL-184, aflibercept, apatinib, bevacizumab, BIBF-1120 brivanib, cediranib, fluocinolone, linifanib, midostaurin, motesanib, pazopanib, pegaptanib, alacizumab pegol, XL-999, XL-647, ramucirumab, ranibizumab, ridoforolimus, sorafenib, tivozanib, vatalanib, VEGF-Trap-Eye, pirfenidone, masitinib, axitinib, dasatinib, linifanib, nilotinib PD 173074, picropodophyllotoxin (PPP), PHA 665752, DMPQ, SU4312, K252a, or sunitinib. In some embodiments, the growth factor is AEE788, CP-751871, BIBW 2992, XL-999, XL-647, catumaxomab, cetuximab, EGF vaccine (CIMAB/Micromet/Biocon/Bioven); erlotinib, icotinib, lapatinib, lapatinib+pazopanib, leflunomide, necitumumab, neratinib, nimotuzumab, panitumumab, pertuzumab, Polyphenon E, trastuzumab, vandetanib, zalutumumab, CNTF, tanezumab, dalotuzumab, AMG-479, figitumumab, rilotumumab, lanreotide, OSI 906, pasireotide, PF-2341066, MetMab, alacizumab pegol, XL-184, aflibercept, apatinib, axitinib, bevacizumab, BIBF-1120 brivanib, cediranib, fluocinolone, linifanib, midostaurin, motesanib, pazopanib, pegaptanib, ramucirumab, ranibizumab, ridoforolimus, sorafenib, tivozanib, VEGF-Trap-Eye, pirfenidone, masitinib, dasatinib, linifanib, nilotinib, PD 173074, PHA 665752, DMPQ, SU4312, K252a, or sunitinib. In some embodiments, the growth factor is AEE788, BIBW 2992, catumaxomab, EGF vaccine (CIMAB/Micromet/Biocon/Bioven), icotinib, leflunomide, XL-999, necitumumab, neratinib, pertuzumab, zalutumumab, CNTF, tanezumab, dalotuzumab, AMG-479, rilotumumab, lanreotide, OSI 906, pasireotide, PF-2341066, alacizumab pegol, XL-184, MetMab, XL-647, aflibercept, apatinib, BIBF-1120 brivanib, fluocinolone, midostaurin, motesanib, pazopanib, ramucirumab, ridoforolimus, tivozanib, vatalanib, VEGF-Trap-Eye, pirfenidone, masitinib, PD 173074, PHA 665752, DMPQ, SU4312, K252a, or nilotinib.

In some embodiments, the growth factor inhibitor is AEE788, AVASTIN/bevacizumab, axitinib, CP-751871, LUCENTIS/ranibizumab, NEXAVAR/sorafenib, pazopanib, SUTENT/sunitinib, ZD6474, canertinib, ERBITUX/cetuximab, TARCEVAJerlotinib, IRESSA/gefitinib, or lapatinib. In some embodiments, the growth factor inhibitor is AEE788, AVASTIN/bevacizumab, axitinib, CP-751871, LUCENTIS/ranibizumab, NEXAVAR/sorafenib, pazopanib, SUTENT/sunitinib, or ZD6474. In some embodiments, the growth factor inhibitor is AEE788 or pazopanib. In some embodiments, the growth factor inhibitor is PD 173074, picropodophyllotoxin (PPP), PHA 665752, DMPQ, SU4312, or K252a. In some embodiments, the growth factor inhibitor is AEE788, AVASTIN/bevacizumab, axitinib, CP-751871, LUCENTIS/ranibizumab, NEXAVAR/sorafenib, pazopanib, SUTENT/sunitinib, ZD6474, PD 173074, PHA 665752, DMPQ, SU4312, or K252a. In some embodiments, the growth factor inhibitor is AEE788, pazopanib, PD 173074, PHA 665752, DMPQ, SU4312, or K252a. In some embodiments, the growth factor inhibitor is gefitinib, PD 173074, picropodophyllotoxin (PPP), PHA 665752, DMPQ, SU4312, or K252a. In some embodiments, the growth factor inhibitor is PD 173074, PHA 665752, DMPQ, SU4312, or K252a. In some embodiments, the growth factor inhibitor is PD 173074, picropodophyllotoxin (PPP), DMPQ, or K252a. In some embodiments, the growth factor inhibitor is PD 173074, DMPQ, or K252a. In some embodiments the growth factor inhibitor is PD 173074, PHA 665752, DMPQ, SU4312, or K252a. In some embodiments the growth factor inhibitor is PD 173074 or DMPQ. In some embodiments, the growth factor inhibitor is gefitinib (IRESSA). In some embodiments, the growth factor inhibitor is erlotinib (TARCEVA). In some embodiments, the growth factor inhibitor is PD 173074. In some embodiments, the growth factor inhibitor is picropodophyllotoxin (PPT). In some embodiments, the growth factor inhibitor is PHA 665752. In some embodiments, the growth factor inhibitor is DMPQ. In some embodiments, the growth factor inhibitor is SU4312. In some embodiments, the growth factor inhibitor is K252a. In some embodiments the growth factor inhibitor is PD 173074, PHA 665752, DMPQ, SU4312, or K252a. In some embodiments, the growth factor inhibitor is not one or more of AG1024, BMS536924, BMS554417, canertinib, EKB-569, Erbitux/cetuximab, Erbitux/IMC-C2225, erlotinib, IGF1 antibodies, IRESSA/gefitinib, lapatinib, mAb 806, matuzuman, MDX-446, nimutozumab, NVP-ADW742, NVP-AEW541, panitumumab, picropodophyllin (PPP), PKI-166, AVASTIN/bevacizumab, LUCENTIS/ranibizumab, NEXAVAR/sorafenib, ZD6474, imatinib, trastuzumab, TheraCIM hR3, 2C4, AE-941, linifanib, cediranib, pagaptanib, dasatinib, semaxinib (SU5416) or EMD 72000. In some embodiments, the growth factor inhibitor is not one or more of the growth factor inhibitors disclosed in WO 07/11962, WO 08/30883, WO 08/30891, WO 08/89272, WO/147418, US 2007/0292883, US 2008/026062, WO 09/064,738, WO 09/073,869, WO 09/064,444, or WO 09/0331117. In some embodiments, the growth factor inhibitor is not one or more of the growth factor inhibitors disclosed in WO 07/11962, WO 08/30883, WO 08/30891, WO 08/89272, WO/147418, US 2007/0292883, or US 2008/026062. In some embodiments, the growth factor inhibitor is not one or more of the growth factor inhibitors disclosed in WO 09/064,738, WO 09/073,869, WO 09/064,444, or WO 09/0331117. In some of these embodiments, the growth factor inhibitor (or a combination of one or more of the growth factor inhibitors described herein) are as described above and the PARP inhibitor is 4-iodo-3-nitrobenzamide or a pharmaceutically acceptable salt thereof. In some of these embodiments, the growth factor inhibitor (or a combination of one or more of the growth factor inhibitors described herein) are as described above and the PARP inhibitor is 4-iodo-3-nitrobenzamide (or metabolite thereof) or a pharmaceutically acceptable salt, isomer, solvate or tautomer thereof.

In other embodiments, the growth factor inhibitor is HGS-TR2J, HGS-ETR2, mapatumumab, edrecolomab, gemtuzumab, alemtuzumab, or rituximab.

In some embodiments, the NSCLC is a metastatic carcinoma. In some embodiments, the NSCLC is a squamous cell carcinoma, an adenocarcinoma, or a large cell carcinoma. In some embodiments, the NSCLC is deficient in homologous recombination DNA repair.

In some embodiments, the treatment comprises a treatment cycle of at least 11 days, wherein on days 1, 4, 8 and 11 of the cycle, the patient receives about 10 to about 100 mg/kg of 4-iodo-3-nitrobenzamide or a molar equivalent of a metabolite thereof. In some embodiments, the treatment comprises a treatment cycle of at least 11 days, wherein on days 4, 8 and 11 of the cycle, the patient receives about 1 to about 50 mg/kg of 4-iodo-3-nitrobenzamide or a molar equivalent of a metabolite thereof. In some embodiments, the treatment comprises a treatment cycle of at least 11 days, wherein on days 1, 4, 8 and 11 of the cycle, the patient receives about 1, 2, 3, 4, 5, 6, 8, or 10, 12, 14, 16, 18, or 20 mg/kg of 4-iodo-3-nitrobenzamide.

Some embodiments described herein provide a method of treating lung cancer in a patient, comprising during a 21 day treatment cycle, on days 1, 4, 8 and 11 of the cycle, administering to the patient about 10 to about 100 mg/kg of 4-iodo-3-nitrobenzamide or a molar equivalent of a metabolite thereof. In some embodiments, the 4-iodo-3-nitrobenzamide is administered orally or as an intravenous infusion.

Some embodiments provide a method of treating lung cancer including but not limited to non-small cell lung cancer in a patient, comprising: (a) testing a sample from the patient for PARP expression; and (b) if the PARP expression exceeds a predetermined level, administering to the patient at least one PARP inhibitor and at least one growth factor inhibitor. In some embodiments, at least one therapeutic effect is obtained, said at least one therapeutic effect being reduction in size of a lung tumor, reduction in metastasis, complete remission, partial remission, pathologic complete response, or stable disease. In some embodiments, an improvement of clinical benefit rate (CBR=CR+PR+SD≧6 months) is obtained as compared to treatment without the PARP inhibitor. In some embodiments, the improvement of clinical benefit rate is at least about 30%, 40%, 50%, or 60%. In some embodiments, the PARP inhibitor is a PARP-1 inhibitor. In other embodiments, the PARP inhibitor is a benzamide or a metabolite thereof. In some embodiments, the benzamide is 4-iodo-3-nitrobenzamide or a metabolite thereof.

In some embodiments, the lung cancer is a metastatic lung cancer. In some embodiments, the lung cancer is at stage I, II or III. In some embodiments, the lung cancer is a non-small cell lung carcinoma (NSCLC). In some embodiments, the NSCLC is a squamous cell carcinoma, an adenocarcinoma, or a large cell carcinoma. In some embodiments, the lung cancer is a small cell lung carcinoma (SCLC). In some embodiments, the lung cancer is deficient in homologous recombination DNA repair. In some embodiments, the lung cancer is a tumor such as, carcinoid tumors (typical or atypical), carcinosarcomas, pulmonary blastomas, or giant or spindle cell carcinomas.

In some embodiments, the PARP inhibitor and/or the growth factor inhibitor may be capable of being present in a variety of physical forms—e.g., free base, salts (especially pharmaceutically acceptable salts), hydrates, polymorphs, solvates, metabolites, etc. Unless otherwise qualified herein, use of a chemical name is intended to encompass all physical forms of the named chemical. For example, recitation of 4-iodo-3-nitrobenzamide, without further qualification, is intended to generically encompass the free base as well as all pharmaceutically acceptable salts, polymorphs, hydrates, and metabolites thereof. Where it is intended to limit the disclosure or claims to a particular physical form of a compound, this will be clear from the context of the passage or claim in which the reference to the compound appears. In some embodiments, the PARP inhibitor and/or growth factor inhibitor (as appropriate given the specific inhibitor and method of administration) are present as a pharmaceutically acceptable salt, solvate, isomer or tautomer thereof. In some embodiments, the PARP inhibitor and/or growth factor inhibitor may be present as a pharmaceutically acceptable salt thereof.

The terms “effective amount” or “pharmaceutically effective amount” refer to a sufficient amount of the agent to provide the desired biological, therapeutic, and/or prophylactic result. That result can be reduction and/or alleviation of the signs, symptoms, or causes of a disease, or any other desired alteration of a biological system including, for example, improved quality of life. For example, an “effective amount” for therapeutic uses is the amount of a nitrobenzamide compound as disclosed herein per se, or a composition comprising the nitrobenzamide compound disclosed herein, required to provide a clinically significant decrease in a disease. An appropriate effective amount in any individual case may be determined by one of ordinary skill in the art using routine experimentation.

By “pharmaceutically acceptable” or “pharmacologically acceptable” is meant a material which is not biologically or otherwise undesirable, i.e., the material may be administered to an individual without causing significant undesirable biological effects or interacting in a deleterious manner with any of the components of the composition in which it is contained.

The term “treating” and its grammatical equivalents as used herein include achieving a therapeutic benefit and/or a prophylactic benefit. By therapeutic benefit is meant eradication or amelioration of the underlying disorder being treated. For example, in a cancer patient, therapeutic benefit includes eradication or amelioration of the underlying cancer. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For prophylactic benefit, a method of the invention may be performed on, or a composition of the invention administered to a patient at risk of developing cancer, or to a patient reporting one or more of the physiological symptoms of such conditions, even though a diagnosis of the condition may not have been made.

### Growth Factor Inhibitors

The term growth factor refers to a naturally occurring protein capable of stimulating cellular growth, proliferation and cellular differentiation. Growth factors are important for regulating a variety of cellular processes. Growth factors typically act as signaling molecules between cells. Examples are cytokines and hormones that bind to specific receptors on the surface of their target cells. They often promote cell differentiation and maturation, which varies between growth factors. For example, bone morphogenic proteins stimulate bone cell differentiation, while fibroblast growth factors and vascular endothelial growth factors stimulate blood vessel differentiation (angiogenesis).

Individual growth factor proteins tend to occur as members of larger families of structurally and evolutionarily related proteins. There are many families of growth factors including but not limited to bone morphogenetic proteins (BMPs), epidermal growth factor (EGF), erythropoietin (EPO), fibroblast growth factor (FGF), Granulocyte-colony stimulating factor (G-CSF), Granulocyte-macrophage colony stimulating factor (GM-CSF), growth differentiation factor-9 (GDF9), hepatocyte growth factor (HGF), insulin-like growth factor (IGF), myostatin, nerve growth factor (NGF) and other neurotrophins, platelet-derived growth factor (PDGF), thrombopoietin (TPO), transforming growth factor alpha(TGF-α), transforming growth factor beta (TGF-β) and vascular endothelial growth factor (VEGF).

In some embodiments, the growth factor is selected from the group consisting of epidermal growth factor (EGF), nerve growth factor (NGF), insulin-like growth factor I (IGF1), hepatocyte growth factor (HGF), vascular endothelial growth factor (VEGF), hepatoma-derived growth factor (HDGF), fibroblast growth factor (FGF), and platelet derived growth factor (PDGF). In some embodiments, the growth factor is selected from the group consisting of epidermal growth factor (EGF), nerve growth factor (NGF), insulin-like growth factor I (IGF1), hepatocyte growth factor (HGF), vascular endothelial growth factor (VEGF), fibroblast growth factor (FGF), and platelet derived growth factor (PDGF). In some embodiments, the growth factor is selected from the group consisting of fibroblast growth factor (FGF (e.g., PD 173074)) and platelet derived growth factor (PDGF (e.g., DMPQ)). Exemplary inhibitors of these growth factors are provided herein.

Exemplary growth factor inhibitors include, but are not limited to, e.g., AEE788, AVASTIN/bevacizumab, axitinib, CP-751871, LUCENTIS/ranibizumab, NEXAVAR/sorafenib, pazopanib, SUTENT/sunitinib, ZD6474, canertinib, ERBITUX/cetuximab, TARCEVA/erlotinib, IRESSA/gefitinib, lapatinib and additional inhibitors described herein.

Growth factors have been increasingly used in the treatment of hematologic and oncologic diseases and cardiovascular diseases including but not limited to neutropenia, myelodysplastic syndrome (MDS), leukemias, aplastic anaemia, bone marrow transplantation, angiogenesis for cardiovascular diseases.

Epidermal Growth Factor Receptor (EGFR)

In some embodiments, the methods of the invention may comprise administering to a patient with cancer, specifically lung cancer, an effective amount of a PARP inhibitor in combination with an inhibitor targeting a growth factor receptor disclosed herein. One example is the epidermal growth factor receptor (EGFR). Exemplary EGFR inhibitors include, e.g., GW-974, BIBW 2992 (also known as: BIBW2992, TOVOK; Boehringer Ingelheim), matuzumab (also known as EMD-7200), MDX-447 (Medarex), catumaxomab (also known as: Removab, triomab-1; Trion Pharma), cetuximab (also known as: Anti-EGFR monoclonal antibody 225, C 225, ERBITUX, IMC-C225; Bristol-Myers Squibb Co.); EGF vaccine (CIMAB/Micromet/Biocon/Bioven); erlotinib (also known as: CP 358774, NSC 718781, OSI 774, R1415, RG1415, TARCEVA; Chugai Pharmaceutical, Genentech Inc.), gefitinib (also known as: IRESSA, ZD-1839, IRESSAt, M-387783, M-537194, M-523595; AstraZeneca plc), icotinib (also known as: BPI-1096, BPI-2009H; Zhejiang Beta Pharma), lapatinib (also known as: 572016, GW2016, GW572016, GW572016F, TYCERB, TYKERB, TYVERB; GlaxoSmithKline), lapatinib+pazopanib (also known as: TYKERB+ARMALA; GlaxoSmithKline), leflunomide (also known as: ARAVA, HWA 486, SU 101; sanofi-aventis), necitumumab (also known as: IMC 11F8, IMC-11F8; ImClone Systems), neratinib (also known as: HKI-272; Pfizer, Inc.), nimotuzumab (also known as: Anti-EGFR mAb hR3, BIOMAB EGFR, h-R3, hR3, OSAG101, TheraCIM, TheraCIM hR3, THERALOC, VECTHIX; Biocon Biopharmaceuticals), panitumumab (also known as: ABX-EGF, E7.6.3, rHuMAb-EGFr, VECTIBIX, panitumimab; Amgen/Takeda), pertuzumab (also known as: 2C4 antibody (Genentech), Omnitarg, R-1273, R1273, RG-1273, RG1273, rhuMAb 2C4), Polyphenon E Ointment (also known as: sinecatechins, VEREGEN; Epitome Pharmaceuticals), trastuzumab (also known as: anti-HER-2 MAb, Genentech, anti-HER-2 MAb, Roche, HER-2 MAb, Genentech, HER-2 MAb, Roche, HERCEPTIN, R-597, R597, RG-597, RG597, rhuMAb HER2, Ro-45-2317), trastuzumab-DM1 (also known as: HERCEPTIN+DM1, Pro-132365, R-3502, R3502, RG-3502, RG3502, T-DM1, trastuzumab-Mcc-DM1; Hoffmann-La Roche), vandetanib (also known as: AZD6474, ZACTIMA, ZD6474, AstraZeneca plc), pelitinib (also known as EKB-569), PF-299804, XL-647 (Exelixis), and zalutumumab (also known as: HUMAX-EGFR; Genmab).

In some embodiments, the EGF inhibitor is BIBW 2992, catumaxomab, EGF vaccine (CIMAB/Micromet/Biocon/Bioven), icotinib, leflunomide, necitumumab, neratinib, PF-299804, zalutumumab. In some embodiments, the EGF inhibitor is BIBW 2992, catumaxomab, cetuximab; MDX-447, EGF vaccine (CIMAB/Micromet/Biocon/Bioven), erlotinib, icotinib, lapatinib, lapatinib+pazopanib, leflunomide, necitumumab, neratinib, nimotuzumab, panitumumab, pertuzumab, Polyphenon E, trastuzumab, PF-299804, vandetanib or zalutumumab.

EGFR is overexpressed in the cells of certain types of human carcinomas including but not limited to lung and breast cancers. Highly proliferating, invasive breast cancer cells often express abnormally high levels of the EGFR, and this is known to control both cell division and migration. The interest in EGFR is further enhanced by the availability and FDA approval of specific EGFR tyrosine kinase inhibitors, for example, gefitinib. Inhibition of EGFR is an important anti-cancer treatment. Examples of EGFR inhibitors include but are not limited to cetuximab, which is a chimeric monoclonal antibody given by intravenous injection for treatment of cancers including but not limited to metastatic colorectal cancer and head and neck cancer. Panitumimab is another example of EGFR inhibitor. It is a humanized monoclonal antibody against EGFR. Panitumimab has been shown to be beneficial and better than supportive care when used alone in patients with advanced colon cancer and is approved by the FDA for this use.

The epidermal growth factor receptor (EGFR; ErbB-1; HER1 in humans) is the cell-surface receptor for members of the epidermal growth factor family (EGF-family) of extracellular protein ligands. The epidermal growth factor receptor is a member of the ErbB family of receptors, a subfamily of four closely related receptor tyrosine kinases: EGFR (ErbB-1), HER2/c-neu (ErbB-2), Her 3 (ErbB-3) and Her 4 (ErbB-4). Mutations affecting EGFR expression or activity could result in cancer. Epidermal growth factor receptor (EGFR) plays a critical role in the control of cellular proliferation, differentiation, and survival. Abnormalities in signaling of the EGFR pathway have been found in a wide range of cancers, including but not limited to carcinomas of the lung, breast, and colon. Inhibitors of EGFR such as gefitinib are used in the treatment of these cancers, particularly non-small cell lung cancers which have mutations within the EGFR gene.

Tyrosine kinase inhibitors are promising agents for the treatment and prevention of human cancers. Tyrosine kinase inhibitors directed against EGFR are the first molecular-targeted agents to be approved in the US and other countries for the treatment of advanced non-small-cell lung cancer after failure of chemotherapy. Some patient characteristics, such as never-smoking, female gender, East Asian origin, adenocarcinoma histology, and bronchioloalveolar subtype, are associated with a greater benefit from treatment with EGFR inhibitors.

Lung cancer is the leading cause of cancer-related death in the Western world and the mortality rate is rapidly increasing in Asia; 1.2 million cancer deaths worldwide were from lung cancer in the year 2002. There are primarily two major types of lung cancer: non-small-cell lung cancer (NSCLC) and small-cell lung cancer. More than 50% of NSCLC patients are candidates for systemic treatment with chemotherapy, either for advanced disease, or as adjuvant or neoadjuvant treatment, in addition to local therapy. Chemotherapy has, however, modest activity in NSCLC and, in the past few years, several drugs that are more specific for cancer cell targets have shown activity in NSCLC. There are two molecular-targeted agents approved for the treatment of advanced NSCLC: gefitinib (IRESSA, AstraZeneca, Wilmington, Del.) and erlotinib (TARCEVA, OSI Pharmaceuticals Inc, Melville, N.Y.). Both agents are small molecules that belong to the quinazolinamine class and inhibit the tyrosine kinase activity of the epidermal growth factor receptor (EGFR) by competing with ATP for the ATP-binding site (Giuseppe Giaccone; Jose Antonio Rodriguez, Nat Clin Pract Oncol. 2005; 2(11):554-561). Besides these two rather selective tyrosine kinase inhibitors (TKIs) of EGFR, other TKIs with a broader spectrum of activity, and monoclonal antibodies to the extracellular domain of the receptor, are also being tested in advanced NSCLC. Among broader spectrum TKIs are lapatinib and canertinib, which have activity on more members of the ErbB family of receptors, and ZD6474 and AEE788, which inhibit the vascular endothelial factor receptor in addition to EGFR. After failure of chemotherapy, gefitinib and erlotinib are able to induce major objective responses in approximately 10% of Caucasian patients and 25-30% of Japanese patients (gefitinib) with NSCLC tumors. The response rate to the EGFR monoclonal antibody cetuximab (ERBITUX, ImClone Systems/Bristol-Myers Squibb) appears similar in the same setting.

Recently, studies have identified gene mutations targeting the kinase domain of the EGFR that are related to the response to inhibitors. Most EGFR mutations predict a higher benefit from treatment compared with wild-type receptors and are correlated with clinical features related to better outcome; some EGFR mutations, however, confer drug resistance. The analysis of material usually available from lung cancer patients, using techniques such as direct sequencing to determine EGFR mutational status, can be technically challenging. In this regard, high EGFR copy number and EGFR protein detected by immunohistochemistry can also be used to select those patients who would benefit from treatment.

Gefitinib (originally coded ZD1839) is a drug used in the treatment of certain types of cancer. Acting in a similar manner to erlotinib (marketed as TARCEVA), gefitinib selectively targets the mutant proteins in malignant cells. It is marketed by AstraZeneca and Teva under the trade name IRESSA. ZD1839 (gefitinib or IRESSA) is an orally active epidermal growth factor receptor (EGFR) tyrosine kinase inhibitor that blocks signal transduction pathways in epithelial cells. Research on gefitinib-sensitive non-small cell lung cancers has shown that a mutation in the EGFR tyrosine kinase domain is responsible for activating anti-apoptotic pathways (Pao W, et al. Proc Nall Acad Sci USA 2004; 101:13306-11; Sordella R, et al. Science 2004; 305:1163-7). These mutations tend to confer increased sensitivity to tyrosine kinase inhibitors such as gefitinib and erlotinib. Of the types of non-small cell lung cancer histologies, adenocarcinoma is the type that most often harbors these mutations. These mutations are more commonly seen in Asians, women, and non-smokers (who also tend to more often have adenocarcinoma). Gefitinib inhibits EGFR tyrosine kinase by binding to the adenosine triphosphate (ATP)-binding site of the enzyme. Thus the function of the EGFR tyrosine kinase in activating the Ras signal transduction cascade is inhibited, and malignant cells are inhibited. Gefitinib is currently only indicated for the treatment of locally advanced or metastatic non-small cell lung cancer (NSCLC) in patients who have previously received chemotherapy. While gefitinib has yet to be proven to be effective in other cancers, there is certainly potential for its use in the treatment of other cancers where EGFR overexpression is involved.

It has been demonstrated that approximately 85% of NSCLC patients who show radiographic responses to treatment with gefitinib or erlotinib have somatic mutations in the EGFR gene (Paez J G, et al. Science 2004; 304:1497-500; Lynch T J, et al. N Engl J Med 2004; 350:2129-39; Pao W, et al. Proc Nall Acad SciUSA 2004; 101:13306-11; Mitsudomi T, et al. J Clin Oncol 2005; 23:2513-20; Han S W, J Clin Oncol 2005; 23:2493-501). These mutations found to date are located in the first four exons that encode the kinase domain (i.e., exons 18-21) and include small overlapping deletions, insertions, and missense mutations. The most common mutations, which account for approximately 85% of the mutations described to date, include deletions in exon 19 and the L858R missense mutation in exon 21 (Han S W, et al. J Clin Oncol 2005; 23: 2493-501; Kosaka T, et al. Cancer Res 2004; 64: 8919-23; Shigematsu H, et al. J Natl Cancer Inst 2005; 97: 339-46; Huang S F, et al. Clin Cancer Res 2004; 10: 8195-203). Autophosphorylation of both of these mutant EGFR proteins is inhibited at concentrations of gefitinib that are 10- to 100-fold lower than those that are necessary to inhibit wild-type EGFR (Paez J G, et al. Science 2004; 304:1497-500; Tracy S. et al., Cancer Res 2004; 64:7241-4). In addition, NSCLC cells with mutant but not wild-type EGFR undergo apoptosis following gefitinib treatment (Paez J G, et al. Science 2004; 304:1497-500; Tracy S. et al., Cancer Res 2004; 64:7241-4).

Overexpression of EGFR is reported to occur in 40%-80% of NSCLC cases (Salomon D S, et al. Crit. Rev Oncol Hematol 1995; 19:183-232), and patients with somatic mutations of the EGFR gene have a significantly higher response rate to treatment with EGFR TKIs than patients with wildtype EGFR (von Eyben F E. Crit. Rev Clin Lab Sci 2006; 43:291-323). Additionally, of those patients achieving a partial response, patients with EGFR mutations show a trend toward a longer duration of response in comparison with patients without EGFR mutations. EGFR gene amplification detected by fluorescence in situ hybridization can also be used to select patients for EGFR tyrosine kinase inhibitor (TKI) therapy (Cappuzzo F, Hirsch F R, Rossi E et al. J Natl Cancer Inst 2005; 97:643-655; Hirsch F R, et al. J Clin Oncol 2005; 23:6838-6845). Higher response rates to EGFR TKIs have also been seen in other patient subgroups, such as women, those with an Asian background, never-smokers, and patients with adenocarcinoma. This may be a result of an association between these clinical characteristics and the EGFR mutations (usually amino acid deletions or substitutions) thought to occur on exons 19 and 21 (von Eyben F E. Crit. Rev Clin Lab Sci 2006; 43:291-323; Lynch T J, Bell D W, Sordella R et al. N Engl J Med 2004; 350:2129-2139).

TARCEVA (erlotinib) is an oral anti-cancer drug developed by OSI Pharmaceuticals, Genentech and Roche. It is a member of the Epidermal Growth Factor Receptor (EGFR) inhibitor class of agents and currently indicated for treatment of Non-Small Cell Lung Cancer (NSCLC) and pancreatic cancer. TARCEVA received US FDA approval for the treatment of NSCLC in 2004 and gained the distinction of being the first EGFR inhibitor to show a survival benefit in lung cancer patients. European approval for treatment of NSCLC in patients failing prior chemotherapy followed in 2005.

On the back of successful phase III trials in pancreatic cancer, TARCEVA has now secured approval for treatment of advanced pancreatic cancer in combination with gemcitabine in chemotherapy-naïve patients in both the US and Europe. As the first new pancreatic cancer therapy for a decade, this represents a major development for this difficult-to-treat disease.

Over expression of EGFR is common in many solid tumors including, but not limited to, colorectal and lung carcinomas as well as cancers of the head and neck. It correlates with increased metastasis, decreased survival and a poor prognosis. EGFR protects malignant tumour cells from the cytotoxic effects of chemotherapy and radiotherapy, making these treatments less effective. TARCEVA works by inhibiting receptor tyrosine kinase activity, the protein product of the EGFR gene. By interfering with cell signalling pathways involved in cell proliferation, inhibition of EGFR-associated tyrosine kinase represents a novel approach to the treatment of solid tumours. TARCEVA is one of several cancer drugs that target EGFR.

Cancers of the lung are of two types: NSCLC and small-cell lung cancer (SCLC). NSCLC is the most common, accounting for around 80% of all lung cancers. It is an aggressive disease, for which overall 5-years survival rates are less than 10%. New forms of treatment are urgently needed for this intractable form of cancer. TARCEVA was explored as a treatment for NSCLC in a series of phase III clinical trials in which it was used either alone or in combination with other anti-cancer agents. These trials followed promising results in phase II studies, which showed a clear survival advantage when TARCEVA was used as monotherapy in patients with chemo-refractory NSCLC. Of the 57 evaluable patients, 51% achieved disease stabilisation and 40% survived for at least 12 months. Survival benefit was subsequently confirmed in the primary registration trial, a phase III randomised, double-blind study in which TARCEVA was compared with placebo in 731 patients with NSCLC who had failed prior chemotherapy. Among patients treated with TARCEVA there was a 42% improvement in median survival and a 45% improvement in one-year survival. Statistically significant improvements were also seen in all secondary endpoints, which included time to symptom deterioration, progression-free survival and response rate. TARCEVA appears generally well tolerated. Only rash and diarrhea occurred with greater frequency in the TARCEVA arm of the primary registration trial in comparison to placebo, a finding consistent with the other trials. Rash is a common side effect of treatment with EGFR-inhibitors and affected 75% of patients in the TARCEVA NSCLC primary registration trial. It has been suggested that rash may serve as a biomarker of potential drug activity.

Because many solid tumours over express EGFR, TARCEVA has therapeutic potential in the treatment of cancers other than just NSCLC. Similar to lung cancer, pancreatic cancer has proved notoriously hard to treat and has an especially poor prognosis. Results of the 450-patient pancreatic cancer trial showed that when administered in combination with gemcitabine, TARCEVA® improved survival (primary endpoint). Combination therapy produced a statistically significant 23.5% improvement in overall survival in patients with locally advanced or metastatic pancreatic cancer compared with gemcitabine alone. Median and one-year survival in the combination treatment arm were 6.4 months and 25.6% respectively, which compared with 5.9 months and 19.7% respectively in those receiving gemcitabine plus placebo. Progression-free survival was also statistically significantly greater in the combination treatment arm. The results in pancreatic cancer showed importantly that TARCEVA had efficacy beyond NSCLC, its first indication. Other indications in which TARCEVA has produced objective evidence of anti-tumor activity in patients failing standard chemotherapy include ovarian cancer as well as cancers of the head and neck.

Vascular Endothelial Growth Factor Receptor (VEGFR)

In some embodiments, the methods of the invention may comprise administering to a patient with cancer an effective amount of a PARP inhibitor in combination with an inhibitor targeting a growth factor receptor, for example, the vascular endothelial growth factor receptor (VEGFR). For example, exemplary VEGF inhibitors include, but are not limited to: aflibercept (also known as: AVE 0005, AVE 005, AVE0005; Bayer Healthcare/Sanofi-Aventis), XL-999, apatinib (also known as: YN-968D1, YN968D1; Advenchen, Inc.), axitinib (also known as: AG-13736, AG-013736, Agouron/Pfizer), vatalanib, bevacizumab (also known as: AVASTIN, R 435, R435, RG435; Genentech), BIBF-1120 (also known as: Vargatef, Boehringer Ingelheim), brivanib (also known as: BMS-582664, BMS-540215, IDDBCP180722; Bristol-Myers Squibb Co), cediranib (also known as: RECENTIN, AZD-2171; AstraZeneca plc), semaxinib (also known as SU5416, Phamacia), fluocinolone (also known as: MEDIDUR; ILUVIEN; Alimera Sciences Inc.), lapatinib, lapatinib+pazopanib (also known as: TYKERB+ARMALA GlaxoSmithKline), linifanib (also known as: ABT-869, HT-1080, RG-3635, RG3635; Hoffmann-La Roche), midostaurin (also known as: 4-N benzoylstaurosporine, 4-N-benzoyl staurosporine, Benzoylstaurosporine, CGP 41251, N-benzoyl-staurosporine, PKC412, PKC412A; Novartis), motesanib (also known as: AMG-706; Amgen, Inc.), OTS-102 (OncoTherapy Science, Inc.), OSI-632 (OSI Pharmaceuticals Inc), AE-941 (also known as: Neovastat; Aeterna Laboratories), pazopanib (also known as: GW-786034, VOTRIENT, ARMALA, 786034, GW-786034B; GlaxoSmithKline), BMS-690514, pegaptanib (also known as: Macuverse (Macugen), EYE-001 (OcuPhor), (OSI; Eyetech/IOMED) NX-1838), ramucirumab (also known as: IMC-2C6, IMC-1121, IMC-1121B; ImClone Systems Inc.), ranibizumab (also known as: Y0317, LUCENTIS, RG-3645; Genentech, Inc., Novartis, Inc), ridoforolimus (also known as: AP-23573, AP-573, Ariad573; deforolimus, MK-8669; Ariad/Merck & Co), sorafenib (also known as: BAY-43-9006; IDDBCP150446, NEXAVAR, BAY-54-9085, Bayer AG, Onyx Pharmaceuticals, Inc.), sunitinib (also known as: sutene, PHA-290940AD, SU-010398, SU-011248, SU-11248J, SU-12662, SUTENT, SU-11248; SUGEN Inc./Pfizer Inc., Pharmacia Corp.), tivozanib (also known as: KRN-951, AV-951, AVEO Pharmaceuticals Inc), vandetanib (also known as: AZD6474, ZACTIMA, ZD6474; AstraZeneca plc), XL-647, VEGF-Trap-Eye (Bayer), alacizumab pegol, SU4312, and XL-184 (also known as: BMS-907351, Bristol-Myers Squibb Co/Exelixus, Inc.).

In some embodiments, the VEGF inhibitor is aflibercept, apatinib, BIBF-1120, brivani, fluocinolone, midostaurin, motesanib, OTS-102, OSI-632, AE-941, vatalanib, pazopanib, BMS-690514, ramucirumab, ridoforolimus, tivozanib, XL-647, XL-999, VEGF-Trap-Eye, alacizumab pegol, SU4312, or XL-184. In some embodiments, the VEGF inhibitor is aflibercept, vatalanib, apatinib, axitinib, bevacizumab, BIBF-1120, brivanib, cediranib, fluocinolone, lapatinib, lapatinib+pazopanib, linifanib, midostaurin, motesanib, semaxinib, OTS-102, OSI-632, AE-941, pazopanib, BMS-690514, pegaptanib, ramucirumab, ranibizumab, ridoforolimus, sorafenib, sunitinib, tivozanib, vandetanib, VEGF-Trap-Eye (Bayer), XL-647, XL-999, alacizumab pegol, SU4312, or XL-184. In some embodiments, the inhibitor is axitinib, bevacizumab, lapatinib, pazopanib, ranibizumab, sorafenib, SU4312, or sunitinib. In some embodiments, the inhibitor is selective for VEGF and PDGF (e.g., SU4312).

VEGF receptors are receptors for vascular endothelial growth factor (VEGF). Vascular endothelial growth factor (VEGF) is an important signaling protein involved in both vasculogenesis (the formation of the embryonic circulatory system) and angiogenesis (the growth of blood vessels from pre-existing vasculature). As its name implies, VEGF activity is restricted mainly to cells of the vascular endothelium, although it does have effects on a limited number of other cell types (e.g., stimulation monocyte/macrophage migration). In vitro, VEGF has been shown to stimulate endothelial cell mitogenesis and cell migration. VEGF also enhances microvascular permeability and is sometimes referred to as vascular permeability factor.

VEGF has been implicated with poor prognosis in breast cancer. Numerous studies show a decreased overall survival and disease-free survival in those tumors overexpressing VEGF. The overexpression of VEGF may be an early step in the process of metastasis, a step that is involved in the “angiogenic” switch. VEGF is also released in rheumatoid arthritis in response to TNF-α, increasing endothelial permeability and swelling and also stimulating angiogenesis (formation of capillaries). Once released, VEGF may elicit several responses. It may cause a cell to survive, move, or further differentiate. Hence, VEGF is a potential target for the treatment of cancer. The first anti-VEGF drug, a monoclonal antibody named bevacizumab, was approved in 2004. Approximately 10-15% of patients benefit from bevacizumab therapy, although biomarkers for bevacizumab efficacy are not yet known.

Anti-VEGF therapies are important in the treatment of certain cancers and in age-related macular degeneration. They can involve monoclonal antibodies such as bevacizumab (AVASTIN), antibody derivatives such as ranibizumab (LUCENTIS), or orally-available small molecules that inhibit the tyrosine kinases stimulated by VEGF: e.g., sunitinib (SUTENT), sorafenib (NEXAVAR), axitinib, and pazopanib.

Bevacizumab, a monoclonal antibody targeting VEGF that was approved for the treatment of colorectal cancer, extended survival in a clinical trial when used in combination with chemotherapy for selected patients with NSCLC with nonsquamous histology and lacking brain metastases or bleeding (Sandler A, Gray R, Perry M C et al. N Engl J Med 2006; 355:2542-2550). Several small-molecule VEGFR TKIs have activity in NSCLC, and additional trials are in progress (Sandler A, Gray R, Perry M C et al. N Engl J Med 2006; 355:2542-2550). These antiangiogenic agents are also being studied in small-cell lung cancer.

Insulin-Like Growth Factor Receptor

In some embodiments, the methods of the invention may comprise administering to a patient with cancer an effective amount of a PARP inhibitor in combination with an inhibitor targeting a growth factor receptor, for example, the insulin-like growth factor receptor (IGFIR). Exemplary IGF inhibitors include, but are not limited to: dalotuzumab (also known as: F-50035, MK-0646, h7C10, A2CHM; Pierre Fabre S A), AMG-479, picropodophyllotoxin (PPP), figitumumab (also known as: CP 751871, CP-751, 871; Pfizer, Inc.), rilotumumab, lanreotide (also known as: dermopeptin, somatuline, BIM-23014C, BN-52030, ipstyl, ITM-014, DC13-116, Angiopeptin; Ipsen, Inc.), OSI 906 (OSI Pharmaceuticals), and pasireotide (also known as: SOM 230, SOM230C (Novartis, Inc.). In some embodiments, the IGF inhibitor may be: dalotuzumab, AMG-479, rilotumumab, lanreotide, OSI 906, or pasireotide.

Activation of the type I insulin-like growth factor receptor (IGFIR) promotes proliferation and inhibits apoptosis in a variety of cell types. Transgenic mice expressing a constitutively active IGFIR or IGF-I develop mammary tumors and increased levels of IGFIR have been detected in primary breast cancers (Yanochko et. al. Breast Cancer Research 2006). It has also been shown that the insulin-like growth factor I receptor (IGFIR) and HER2 display important signaling interactions in breast cancer. Specific inhibitors of one of these receptors may cross-inhibit the activity of the other. Targeting both receptors give the maximal inhibition of their downstream extracellular signal-regulated kinase 1/2 and AKT signaling pathways. Hence, such drug combinations may be clinically useful and may be beneficial even in tumors in which single drugs are inactive, as exemplified by the effect of the HER2/IGFIR inhibitor combination in HER2 nonoverexpressing MCF7 cells (Chakraborty A K, et. al, Cancer Res. 2008 Mar. 1; 68(5):1538-45). One example of an IGF1R inhibitor is CP-751871. CP-751871 is a human monoclonal antibody that selectively binds to IGF1R, preventing IGF1 from binding to the receptor and subsequent receptor autophosphorylation. Inhibition of IGF1R autophosphorylation may result in a reduction in receptor expression on tumor cells that express IGF1R, a reduction in the anti-apoptotic effect of IGF, and inhibition of tumor growth. IGF is a receptor tyrosine kinase expressed on most tumor cells and is involved in mitogenesis, angiogenesis, and tumor cell survival.

Nerve Growth Factor Receptor (NGFR)

In some embodiments, the methods of the invention may comprise administering to a patient with cancer an effective amount of a PARP inhibitor in combination with an inhibitor targeting a growth factor receptor, for example, the nerve growth factor receptor (NGFR). Exemplary NGF inhibitors include, but are not limited to: CNTF (also known as: NTC-201E, NTC-501; Neurotech), K252a (also known as: (9S-(9α,10β,12α))-2,3,9,10,11,12-hexahydro-10-hydroxy-10-(methoxycarbonyl)-9-methyl-9,12-epoxy-1H-diindolo[1,2,3-fg:3′,2′,1′-kl]pyrrolo[3,4-i][1,6]benzodiazocin-1-one; LC Labs) and tanezumab (also known as: PF 4383119, PF-04383119, PF-4383119, RI 624, RN 624, RN624). In some embodiments, the NGF inhibitor is K252a.

Nerve growth factor (NGF) is a small secreted protein which induces the differentiation and survival of particular target neurons (nerve cells). NGF is critical for the survival and maintenance of sympathetic and sensory neurons. NGF is released from the target cells, binds to and activates its high affinity receptor (TrkA), and is internalized into the responsive neuron. NGF and its receptor are aberrantly expressed in the liver of the patients troubled with liver cirrhosis and/or hepatocellular carcinoma. Study has shown that nerve growth factor (NGF), the prototypic neurotrophin, can be targeted in breast cancer to inhibit tumor cell proliferation, survival, and metastasis (Eric Adriaenssens et al. Cancer Research 68, 346-351, Jan. 15, 2008).

NGF has antiproliferative and differentiating effects on adenomas of neuroendocrine origin. Cell lines derived from small-cell lung carcinoma (SCLC), a very aggressive neuroendocrine tumor, express NGF receptors. Chronic exposure of NCI-N-592 and GLC8 SCLC cell lines to NGF inhibits their proliferation rate both in vitro and in vivo, prevents their anchorage-independent clonal growth in soft agar, impairs their invasive capacity in vitro, and abolishes their tumorigenic potential in nude mice (Cristina Missale et al. PNAS Apr. 28, 1998 vol. 95 no. 9 5366-5371). The proliferative response of SCLC cell lines to nicotine was also remarkably impaired by in vitro NGF treatment. Furthermore, NGF treatment activates in SCLC cell lines the expression and secretion of NGF. NGF thus reverts SCLC cell lines to a noninvasive, nontumorigenic phenotype that does not respond to nicotine and produces NGF.

Hepatocyte Growth Factor Receptor (HGFR)

In some embodiments, the methods of the invention may comprise administering to a patient with cancer an effective amount of a PARP inhibitor in combination with an inhibitor targeting a growth factor receptor, for example, the hepatocyte growth factor receptor (HGFR). Exemplary HGF inhibitors include, but are not limited to: PF-2341066 (also known as: PF-02341066; Pfizer, Inc.), MetMab, PHA 665752 (Norcris Bioscience), and XL-184 (also known as: BMS-907351; Exelixis Inc/Bristol-Myers Squibb Co.). In some embodiments, the growth factor inhibitor is PHA 665752.

The hepatocyte growth factor receptor (HGFR), also called c-Met, is activated by HGF and stimulates proliferation of hepatocytes and other cell types. Mutated forms of the HGF receptor are associated with oncogenesis and metastasis, making the HGF receptor a potential therapeutic target for cancer drugs. Changes in cell motility, cell shape, adhesion, resistance to apoptosis, and anchorage independent growth all contribute to the role of c-Met in cancer. Overexpressed or activated hepatocyte growth factor receptor, encoded by the MET proto-oncogene, has been found in the majority of colorectal carcinomas (Rasola A et al. Oncogene. 2007 Feb. 15; 26(7):1078-87).

Hepatoma-Derived Growth Factor (HDGF)

In some embodiments, the methods of the invention may comprise administering to a patient with cancer an effective amount of a PARP inhibitor in combination with an inhibitor targeting a growth factor receptor, for example, the hepatoma-derived growth factor receptor (HDGFR).

Hepatoma-derived growth factor (HDGF) is a heparin-binding protein purified from the conditioned medium of the human well-differentiated hepatocellular carcinoma (HCC) cell line, HuH-7, which can proliferate autonomously in a serum-free chemically-defined medium (Nakamura et al., 1989, 1994). Hepatoma-derived growth factor is highly expressed in several cancer cells (Nakamura et al., 1994, 2002; Mori et al., 2004; Lepourcelet et al., 2005). This growth factor is also more highly expressed in various fetal organs than in adult organs (Oliver and Al-Awqati, 1998; Everett et al., 2000; Enomoto et al., 2002). In the fetus, HDGF is abundantly expressed in the liver, heart, kidney, lungs, and gut. Thus, HDGF is one of the developmentally regulated genes which are abundantly expressed in cancer cells.

PARP Inhibitors:

In some embodiments, the present invention provides a method of treating lung cancer, including all subtypes of lung cancer, by administering to a subject in need thereof at least one PARP inhibitor in combination with a growth factor inhibitor. In other embodiments, the present invention provides a method of treating non-small cell lung cancer (NSCLC) by administering to a subject in need thereof at least one PARP inhibitor in combination with at least one growth factor inhibitor described herein.

Not intending to be limited to any particular mechanism of action, the compounds described herein are believed to have anti-cancer properties due to the modulation of activity of a poly (ADP-ribose) polymerase (PARP). This mechanism of action is related to the ability of PARP inhibitors to bind PARP and decrease its activity. PARP catalyzes the conversion of β-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 June 2001, pp. 97-110(14)). Poly(ADP-ribose) polymerase 1 (PARP1) is a key molecule in the repair of DNA single-strand breaks (SSBs) (de Murcia J, et al. 1997, Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921).

BRCA1 and BRCA2 act as an integral component of the homologous recombination machinery (HR) (Narod S A, Foulkes W D (2004) Nat Rev Cancer 4:665-676; Gudmundsdottir K, Ashworth A (2006) Oncogene 25:5864-5874).

Cells defective in BRCA1 or BRCA2 have a defect in the repair of double-strand breaks (DSB) by the mechanism of homologous recombination (HR) by gene conversion (Farmer H, et al. (2005) Nature 434:917-921; Narod S A, Foulkes W D (2004) Nat Rev Cancer 4:665-676; Gudmundsdottir K, Ashworth A (2006) Oncogene 25:5864-5874; Helleday T, et al. (2008) Nat Rev Cancer 8:193-204). Deficiency in either of the breast cancer susceptibility proteins BRCA1 or BRCA2 induces profound cellular sensitivity to the inhibition of poly(ADP-ribose) polymerase (PARP) activity, resulting in cell cycle arrest and apoptosis. It has been reported that the critical role of BRCA1 and BRCA2 in the repair of double-strand breaks by homologous recombination (HR) is the underlying reason for this sensitivity, and the deficiency of RAD51, RAD54, DSS1, RPA1, NBS1, ATR, ATM, CHK1, CHK2, FANCD2, FANCA, or FANCC induces such sensitivity (McCabe N. et. al. Deficiency in the repair of DNA damage by homologous recombination and sensitivity to poly(ADP-ribose) polymerase inhibition, Cancer research 2006, vol. 66, 8109-8115). It has been proposed that PARP1 inhibition can be a specific therapy for cancers with defects in BRCA1/2 or other HR pathway components (Helleday T, et al. (2008) Nat Rev Cancer 8:193-204). Triple-negative tumors account for 15% of all breast cancers and frequently harbor defects in DNA double-strand break repair through homologous recombination (HR), such as BRCA1 dysfunction (Rottenberg S, et. al. Proc Natl Acad Sci USA. 2008 Nov. 4; 105(44):17079-84).

Inhibiting the activity of a PARP molecule includes reducing the activity of these molecules. The term “inhibits” and its grammatical conjugations, such as “inhibitory,” is not intended to require complete reduction in PARP activity. In some embodiments, such reduction is at least about 50%, at least about 75%, at least about 90%, or at least about 95% of the activity of the molecule in the absence of the inhibitory effect, e.g., in the absence of an inhibitor, such as a nitrobenzamide compound of the invention. In some embodiments, inhibition refers to an observable or measurable reduction in activity. In treatment some scenarios, the inhibition is sufficient to produce a therapeutic and/or prophylactic benefit in the condition being treated. The phrase “does not inhibit” and its grammatical conjugations does not require a complete lack of effect on the activity. For example, it refers to situations where there is less than about 20%, less than about 10%, and preferably less than about 5% of reduction in PARP activity in the presence of an inhibitor such as a nitrobenzamide compound of the invention.

Poly (ADP-ribose) polymerase (PARP) is an essential enzyme in DNA repair, thus playing a potential role in chemotherapy resistance. Targeting PARP potentially is thought to inhibit cell proliferation and/or interrupt DNA repair, thereby enhancing NDA damaging agent-mediated, taxane mediated, antimetabolite mediated, topoisomerase inhibitor-mediated, and growth factor receptor inhibitor-mediated, (e.g., EGFR inhibitor-mediated, FGFR inhibitor mediated, VEGFR inhibitor-mediated, HGFR inhibitor-mediated, PDFGR inhibitor-mediated, HDGFR inhibitor-mediated, or IGF1R inhibitor-mediated), and/or platinum complex mediated-DNA replication and/or repair in cancer cells. PARP inhibitors may also be highly active against cancers with impaired function of BRCA 1 and BRCA2 or those patients with other DNA repair pathway defects.

PARP inhibitors have potential therapeutic benefit when used independently in the treatment of various diseases such as, myocardial ischemia, stroke, head trauma, and neurodegenerative disease, and as an adjunct therapy with other agents including chemotherapeutic agents, radiation, oligonucleotides, or antibodies in cancer therapy. Without limiting the scope of the present embodiments, it shall be understood that various PARP inhibitors are known in the art and are all within the scope of the present embodiments. Some of the examples of PARP inhibitors are disclosed herein but they are not in any way limiting to the scope of the present description.

A great preponderance of PARP inhibitors have been designed as analogs of benzamides, which bind competitively with the natural substrate NAD in the catalytic site of PARP. The PARP inhibitors include, but are not limited to, benzamides, cyclic benzamides, quinolones and isoquinolones and benzopyrones (U.S. Pat. No. 5,464,871, U.S. Pat. No. 5,670,518, U.S. Pat. No. 6,004,978, U.S. Pat. No. 6,169,104, U.S. Pat. No. 5,922,775, U.S. Pat. No. 6,017,958, U.S. Pat. No. 5,736,576, and U.S. Pat. No. 5,484,951, all incorporated herein in their entirety). The PARP inhibitors include a variety of cyclic benzamide analogs (i.e., lactams) which are potent inhibitors at the NAD site. Other PARP inhibitors include, but are not limited to, benzimidazoles and indoles (EP 841924, EP 1127052, U.S. Pat. No. 6,100,283, U.S. Pat. No. 6,310,082, US 2002/156050, US 2005/054631, WO 05/012305, WO 99/11628, and US 2002/028815). A number of low-molecular-weight inhibitors of PARP have been used to elucidate the functional role of poly ADP-ribosylation in DNA repair. In cells treated with alkylating agents, the inhibition of PARP leads to a marked increase in DNA-strand breakage and cell killing (Durkacz et al, 1980, Nature 283: 593-596; and Berger, N. A., 1985, Radiation Research, 101: 4-14). Subsequently, such inhibitors have been shown to enhance the effects of radiation response by suppressing the repair of potentially lethal damage (Ben-Hur et al, 1984, British Journal of Cancer, 49 (Suppi. VI): 34-42; and Schlicker et al, 1999, Int. J. Radiat. Biol. 75: 91-100). PARP inhibitors have been reported to be effective in radio sensitizing hypoxic tumor cells (U.S. Pat. Nos. 5,032,617, 5,215,738 and 5,041,653). Furthermore, PARP knockout (PARP −/−) animals exhibit genomic instability in response to alkylating agents and γ-irradiation (Wang et al, 1995, Genes Dev., 9: 509-520; and Menissier de Murcia et al, 1997, Proc. Natl. Acad. Sci. USA, 94: 7303-7307).

Oxygen radical DNA damage that leads to strand breaks in DNA, which are subsequently recognized by PARP, is a major contributing factor to such disease states as shown by PARP inhibitor studies (Cosi et al, 1994, J. Neurosci. Res., 39: 38-46; and Said et al, 1996, Proc. Natl. Acad. Sci. U.S.A., 93: 4688-4692). It has also been demonstrated that efficient retroviral infection of mammalian cells is blocked by the inhibition of PARP activity. Such inhibition of recombinant retroviral vector infections was shown to occur in various different cell types (Gaken et al, 1996, J. Virology, 70(6): 3992-4000). Inhibitors of PARP have thus been developed for the use in anti-viral therapies and in cancer treatment (WO91/18591). Moreover, PARP inhibition has been speculated to delay the onset of aging characteristics in human fibroblasts (Rattan and Clark, 1994, Biochem. Biophys. Res. Comm., 201(2): 665-672). This may be related to the role that PARP plays in controlling telomere function (d'Adda di Fagagna et al, 1999, Nature Gen., 23(1): 76-80).

PARP inhibitors may possess the following structural characteristics: 1) amide or lactam functionality; 2) an NH proton of this amide or lactam functionality could be conserved for effective bonding; 3) an amide group attached to an aromatic ring or a lactam group fused to an aromatic ring; 4) optimal cis-configuration of the amide in the aromatic plane; and 5) constraining mono-aryl carboxamide into heteropolycyclic lactams (Costantino et al., 2001, J Med. Chem., 44:3786-3794). Virag et al., 2002, Pharmacol Rev., 54:375-429, 2002 summarizes various PARP inhibitors. Some of the examples of PARP inhibitors include, but are not limited to, isoquinolinone and dihydrolisoquinolinone (for example, U.S. Pat. No. 6,664,269, and WO 99/11624), nicotinamide, 3-aminobenzamide, monoaryl amides and bi-, tri-, or tetracyclic lactams, phenanthridinones (Perkins et al., 2001, Cancer Res., 61:4175-4183), 3,4-dihydro-5-methyl-isoquinolin-1(2H)-one and benzoxazole-4-carboxamide (Griffin et al., 1995, Anticancer Drug Des, 10:507-514; Griffin et al., 1998, J Med Chem, 41:5247-5256; and Griffin et al., 1996, Pharm Sci, 2:43-48), dihydroisoquinolin-1(2H)-nones, 1,6-naphthyridine-5(6H)-ones, quinazolin-4(3H)-ones, thieno[3,4-c]pyridin-4(5H)ones and thieno[3,4-d]pyrimidin-4(3H)ones, 1,5-dihydroxyisoquinoline, and 2-methyl-quinazolin-4[3H]-one (Yoshida et al., 1991, J Antibiot (Tokyo) 44:111-112; Watson et al., 1998, Bioorg Med. Chem., 6:721-734; and White et al., 2000, J Med. Chem., 43:4084-4097), 1,8-Napthalimide derivatives and (5H)phenanthridin-6-ones (Banasik et al., 1992, J Biol Chem, 267:1569-1575; Watson et al., 1998, Bioorg Med. Chem., 6:721-734; Soriano et al., 2001, Nat. Med., 7:108-113; Li et al., 2001, Bioorg Med Chem. Lett., 11:1687-1690; and Jagtap et al., 2002, Crit. Care Med., 30:1071-1082), tetracyclic lactams, 1,11b-dihydro-[2H]benzopyrano[4,3,2-de]isoquinolin-3-one, 1-methyl-4-phenyl-1,2,3,6-tetrahydropyridine (MPTP) (Zhang et al., 2000, Biochem Biophys Res Commun., 278:590-598; and Mazzon et al., 2001, Eur J Pharmacol, 415:85-94). Other examples of PARP inhibitors include, but are not limited to, those detailed in the patents: U.S. Pat. No. 5,719,151, U.S. Pat. No. 5,756,510, U.S. Pat. No. 6,015,827, U.S. Pat. No. 6,100,283, U.S. Pat. No. 6,156,739, U.S. Pat. No. 6,310,082, U.S. Pat. No. 6,316,455, U.S. Pat. No. 6,121,278, U.S. Pat. No. 6,201,020, U.S. Pat. Nos. 6,235,748, 6,306,889, U.S. Pat. No. 6,346,536, U.S. Pat. No. 6,380,193, U.S. Pat. No. 6,387,902, U.S. Pat. No. 6,395,749, U.S. Pat. No. 6,426,415, U.S. Pat. No. 6,514,983, U.S. Pat. No. 6,723,733, U.S. Pat. No. 6,448,271, U.S. Pat. No. 6,495,541, U.S. Pat. No. 6,548,494, U.S. Pat. No. 6,500,823, U.S. Pat. No. 6,664,269, U.S. Pat. No. 6,677,333, U.S. Pat. No. 6,903,098, U.S. Pat. No. 6,924,284, U.S. Pat. No. 6,989,388, U.S. Pat. No. 6,277,990, U.S. Pat. No. 6,476,048, and U.S. Pat. No. 6,531,464. Additional examples of PARP inhibitors include, but are not limited to, those detailed in the patent application publications: US 2004198693A1, US 2004034078A1, US 2004248879A1, US 2004249841A1, US 2006074073A1, US 2006100198A1, US 2004077667A1, US 2005080096A1, US 2005171101A1, US 2005054631A1, WO 05054201A1, WO 05054209A1, WO 05054210A1, WO 05058843A1, WO 06003146A1, WO 06003147A1, WO 06003148A1, WO 06003150A1, and WO 05097750A1.

In some embodiments, the PARP inhibitor is selected from the group consisting of benzamide, quinolone, isoquinolone, benzopyrone, cyclic benzamide, benzimidazole and indole, or metabolites of the PARP inhibitors. In one embodiment, the PARP inhibitor is 4-Iodo-3-nitrobenzamide (BA).

4-Iodo-3-nitrobenzamide (BA) is a small molecule that acts on tumor cells without exerting toxic effects in normal cells. BA is believed to achieve its anti-neoplastic effect by inhibition of PARP. BA is very lipophilic and distributes rapidly and widely into tissues, including the brain and cerebrospinal fluid (CSF). It is active against a broad range of cancer cells in vitro, including against drug resistant cell lines. The person skilled in the art will recognize that BA may be administered in any pharmaceutically acceptable form, e.g., as a pharmaceutically acceptable salt, solvate, or complex. Additionally, as BA is capable of tautomerizing in solution, the tautomeric form of BA is intended to be embraced by the term BA (or the equivalent 4-iodo-3-nitrobenzamide), along with the salts, solvates or complexes. In some embodiments, BA may be administered in combination with a cyclodextrin, such as hydroxypropylbetacyclodextrin. However, one skilled in the art will recognize that other active and inactive agents may be combined with BA; and recitation of BA will, unless otherwise stated, include all pharmaceutically acceptable forms thereof.

Basal-like breast cancers have a high propensity to metastasize to the brain; and BA is known to cross the blood-brain barrier. While not wishing to be bound by any particular theory, it is believed that BA achieves its anti-neoplastic effect by inhibiting the function of PARP. In some embodiments, the cancer is an adrenal cortical cancer, anal cancer, aplastic anemia, bile duct cancer, bladder cancer, bone cancer, bone metastasis, CNS tumors, peripheral CNS cancer, Castleman's Disease, cervical cancer, childhood Non-Hodgkin's lymphoma, colon and rectum cancer, esophagus cancer, Ewing's family of tumors, eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, vaginal cancer, vulvar cancer, Waldenstrom's macroglobulinemia or cancers of viral origin. In some embodiments, the cancer is lung cancer. In some embodiments, the lung cancer is a metastatic lung cancer. In some embodiments, the lung cancer is at stage I, II or III. In some embodiments, the lung cancer is at stage I. In some embodiments, the lung cancer is at stage II. In some embodiments, the lung cancer is at stage III. In some embodiments, the lung cancer is a non-small cell lung carcinoma (NSCLC). In some embodiments, the lung cancer is a small cell lung carcinoma (SCLC). In some embodiments, the lung cancer is deficient in homologous recombination DNA repair.

The dosage of PARP inhibitor may vary depending upon the patient age, height, weight, overall health, etc. In some embodiments, the dosage of BA is in the range of about 1 mg/kg to about 100 mg/kg, about 2 mg/kg to about 50 mg/kg, about 2 mg/kg, about 4 mg/kg, about 6 mg/kg, about 8 mg/kg, about 10 mg/kg, about 12 mg/kg, about 15 mg/kg, about 20 mg/kg, about 25 mg/kg, about 30 mg/kg, about 35 mg/kg, about 40 mg/kg, about 50 mg/kg, about 60 mg/kg, about 75 mg/kg, about 90 mg/kg, about 1 to about 25 mg/kg, about 2 to about 70 mg/kg, about 4 to about 100 mg, about 4 to about 25 mg/kg, about 4 to about 20 mg/kg, about 50 to about 100 mg/kg or about 25 to about 75 mg/kg. BA may be administered intravenously, e.g., by IV infusion over about 10 to about 300 minutes, about 30 to about 180 minutes, about 45 to about 120 minutes or about 60 minutes (i.e., about 1 hour). In some embodiments, BA may alternatively be administered orally. In this context, the term “about” has its normal meaning of approximately. In some embodiments, about means±10% or ±5%.

The synthesis of BA (4-iodo-3-nitrobenzamide) is described in U.S. Pat. No. 5,464,871, which is incorporated herein by reference in its entirety. BA may be prepared in concentrations of 10 mg/mL and may be packaged in a convenient form, e.g., in 10 mL vials.

BA Metabolites:

As used herein “BA” means 4-iodo-3-nitrobenzamide; “BNO” means 4-iodo-3-nitrosobenzamide; “BNHOH” means 4-iodo-3-hydroxyaminobenzamide.

Precursor compounds useful in the present invention are of Formula (Ia):

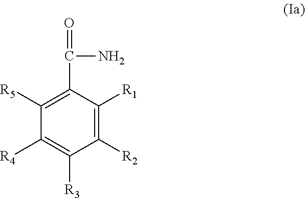

wherein R₁, R₂, R₃, R₄, and R₅ are, independently selected from the group consisting of hydrogen, hydroxy, amino, nitro, nitroso, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents is always nitro, and at least one substituent positioned adjacent to a nitro is always iodo, and pharmaceutically acceptable salts, solvates, isomers, tautomers, metabolites, analogs, or pro-drugs thereof. R₁, R₂, R₃, R₄, and R₅ can also be a halide such as chloro, fluoro, or bromo substituents. In some embodiments, at least one of the R₁, R₂, R₃, R₄, and R₅ substituents is always nitro or nitroso and at least one substituent positioned adjacent to the nitro or nitroso is always iodo. In some embodiments, the compound of formula Ia is a compound of formula IA or a metabolite or pharmaceutically acceptable salt, solvate, isomer, or tautomer thereof. In some embodiments, at least one of the R₁, R₂, R₃, R₄, and R₅ substituents is always nitro or nitroso and at least one substituent positioned adjacent to the nitro or nitroso is always iodo. In some embodiments, the compound of formula Ia is a compound of formula IA or pharmaceutically acceptable salt, solvate, isomer, or tautomer thereof.

A preferred precursor compound of Formula Ia is:

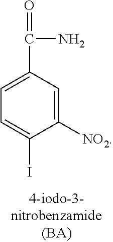

In some embodiments, the compound is 4-iodo-3-nitrobenazmide or a pharmaceutically acceptable salt, solvate, isomer, or tautomer thereof. In some embodiments, the compound is 4-iodo-3-nitrobenazmide or a metabolite (e.g., BNO), or pharmaceutically acceptable salt, solvate, isomer, or tautomer thereof.

Some metabolites useful in the present invention are of the Formula (IIa):

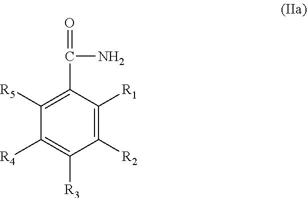

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, and R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄, and R₅ group that is a nitroso, hydroxyamino, hydroxy or amino group. In some embodiments, the compounds of (2) are such that the iodo the iodo group is always adjacent a R₁, R₂, R₃, R₄, and R₅ group that is a nitroso, hydroxyamino, or amino group.

The following compositions are preferred metabolite compounds, each represented by a chemical formula:

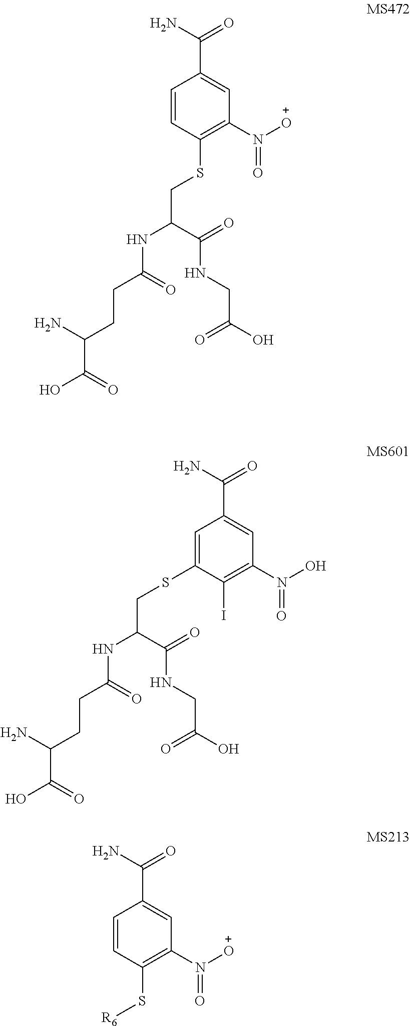

R₆ is selected from a group consisting of hydrogen, alkyl(C₁-C₈), alkoxy (C₁-C₈), isoquinolinones, indoles, thiazole, oxazole, oxadiazole, thiphene, or phenyl.

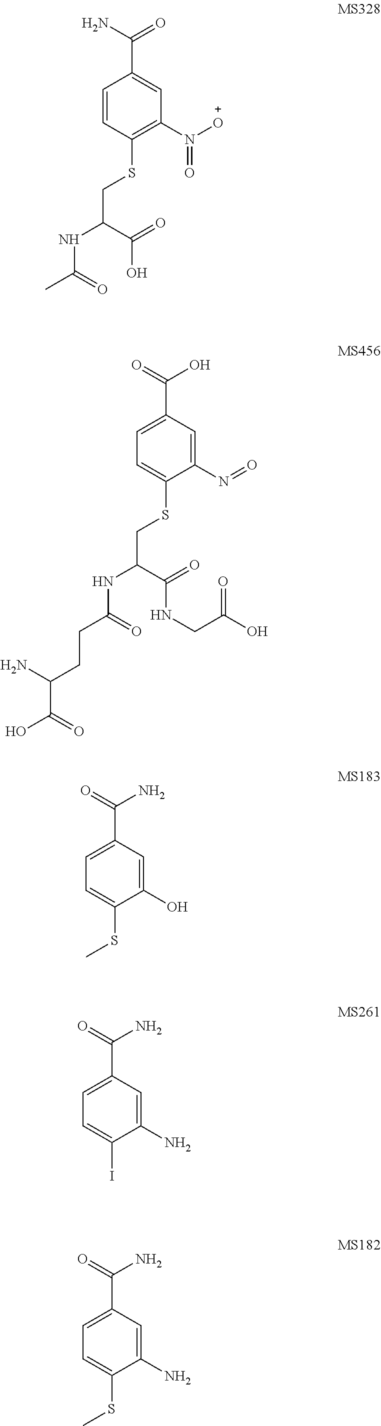

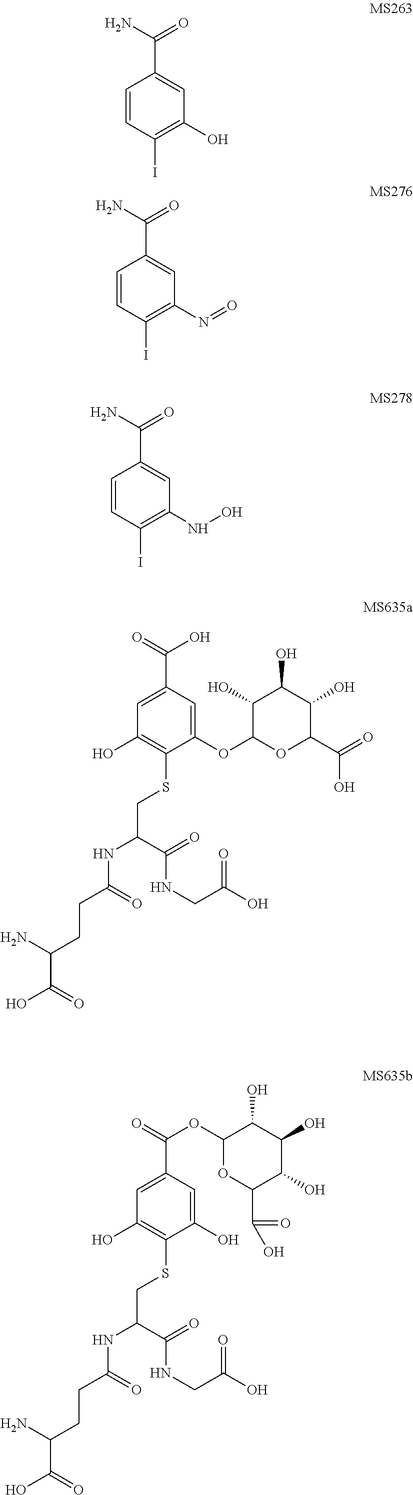

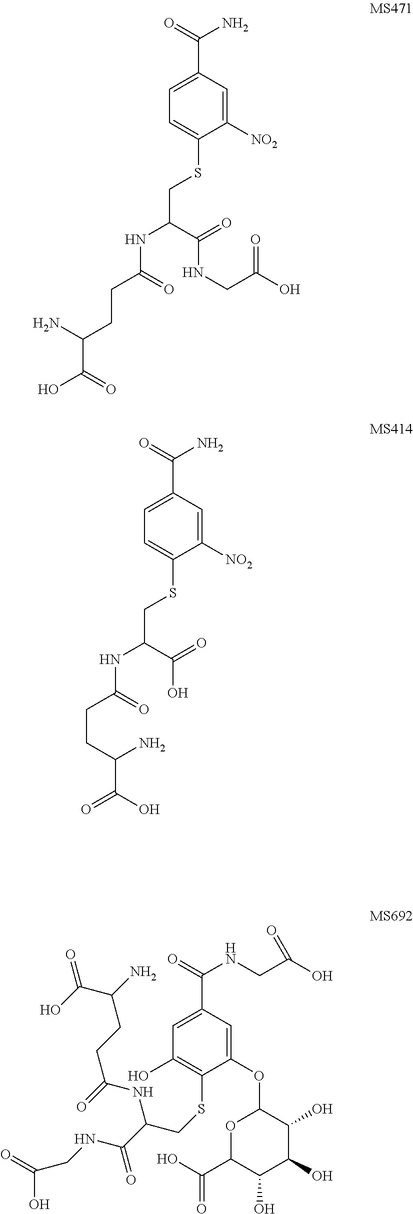

While not being limited to any one particular mechanism, the following provides an example for MS292 metabolism via a nitroreductase or glutathione conjugation mechanism:

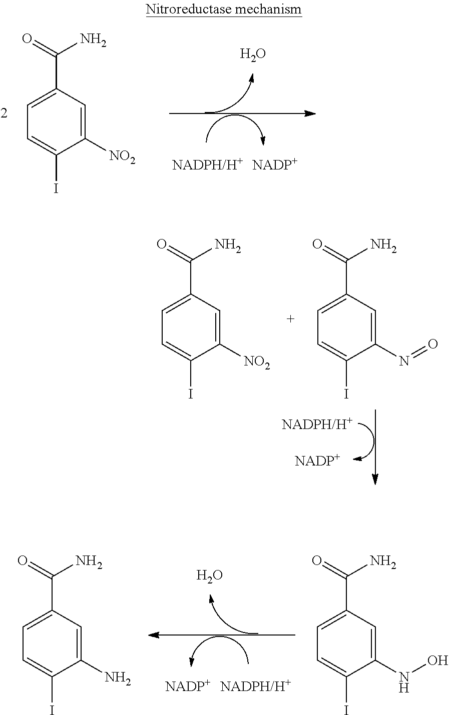

BA Glutathione Conjugation and Metabolism:

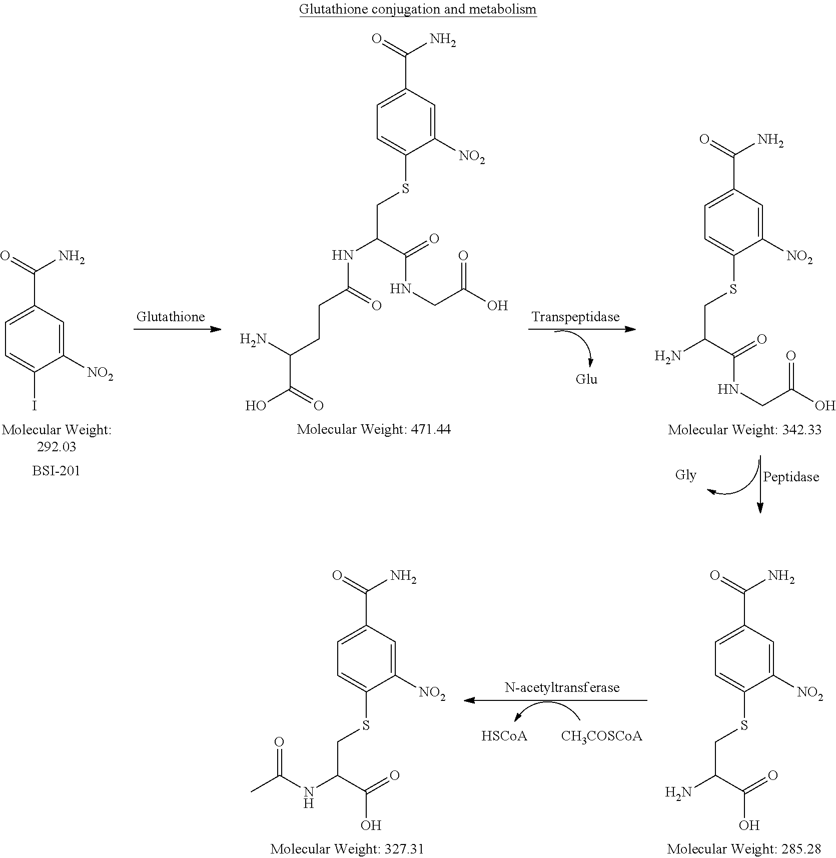

The present invention provides for the use of the aforesaid nitrobenzamide metabolite compounds for the treatment of various cancers, including lung cancer.

It has been reported that nitrobenzamide metabolite compounds have selective cytotoxicity upon malignant cancer cells but not upon non-malignant cancer cells. See Rice et al., Proc. Natl. Acad. Sci. USA 89:7703-7707 (1992), incorporated herein in it entirety. In one embodiment, the nitrobenzamide metabolite compounds utilized in the methods of the present invention may exhibit more selective toxicity towards tumor cells than non-tumor cells.

In some embodiments, the invention provides a method of treating lung cancer by administering to a subject in need thereof at least one PARP inhibitor in combination with at least one growth factor inhibitor. In some embodiments, the metabolites according to the invention are administered to a patient in need of such treatment in conjunction with chemotherapy with at least one antimetabolite (e.g., one of the citabines, such as gemcitabine) and at least one platinum complex (e.g., carboplatin, cisplatin, etc.) In other embodiments, the metabolites according to the invention are thus administered to a patient in need of such treatment in conjunction with chemotherapy with at least one taxane (e.g., paclitaxel or docetaxel) in addition to at least one platinum complex (e.g., carboplatin, cisplatin, etc.) The dosage range for such metabolites may be in the range of about 0.0004 to about 0.5 mmol/kg (millimoles of metabolite per kilogram of patient body weight), which dosage corresponds, on a molar basis, to a range of about 0.1 to about 100 mg/kg of BA. Other effective ranges of dosages for metabolites are 0.0024-0.5 mmol/kg and 0.0048-0.25 mmol/kg. Such doses may be administered on a daily, every-other-daily, twice-weekly, weekly, bi-weekly, monthly or other suitable schedule. Essentially the same modes of administration may be employed for the metabolites as for BA—e.g., oral, i.v., i.p., etc.

Other PARP inhibitors

Also contemplated by the present invention includes benzopyrone compounds of formula II, which may be used in combination with a growth factor inhibitor in the methods described herein. The benzopyrone compounds of formula II are,

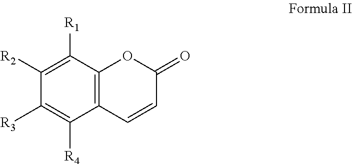

wherein R₁, R₂, R₃, and R₄ are independently selected from the group consisting of H, halogen, optionally substituted hydroxy, optionally substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite, or prodrug thereof (U.S. Pat. No. 5,484,951 is incorporated herein by reference in its entirety).

Some embodiments employ a compound having the chemical formula:

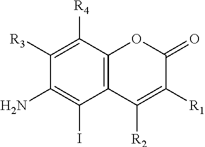

wherein R₁, R₂, R₃, or R₄ are each independently selected from the group consisting of hydrogen, hydroxy, amino, (C₁-C₆) alkyl, (C₁-C₆) alkoxy, (C₃-C₇) cycloalkyl, halo and phenyl and pharmaceutically acceptable salts thereof, wherein at least three of the four R₁, R₂, R₃, or R₄ substituents are always hydrogen.

Some embodiments employ a compound having the chemical formula:

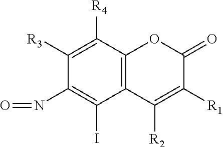

wherein R₁, R₂, R₃, or R₄ are each independently selected from the group consisting of hydrogen, hydroxy, amino, (C₁-C₆) alkyl, (C₁-C₆) alkoxy, (C₃-C₇) cycloalkyl, halo and phenyl and pharmaceutically acceptable salts thereof, wherein at least three of the four R₁, R₂, R₃, or R₄ substituents are always hydrogen.

Some embodiments employ a compound of the chemical formula:

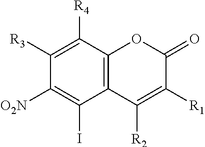

wherein R₁, R₂, R₃, or R₄, are each independently selected from the group consisting of hydrogen, hydroxy, amino, (C₁-C₆) alkyl, (C₁-C₆) alkoxy, (C₃-C₇) cycloalkyl, halo and phenyl, wherein at least three of the four R₁, R₂, R₃, or R₄ substituents are always hydrogen.

One embodiment relates to the following benzopyrone compound of formula II

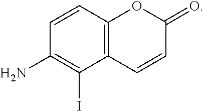

In yet another embodiment, the compound used in the methods described herein is

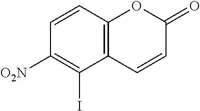

Further details regarding the benzopyrone compounds are in U.S. Pat. No. 5,484,951, which is herein incorporated by reference in its entirety.

It is likely that the most potent and effective PARP inhibitors (i.e., the likely candidates for drug development) are not yet available in the scientific literature but rather are undergoing clinical trials or may ultimately emerge in the various databases of published patents and pending patent applications. All such PARP inhibitors are within the scope of the present embodiments. In addition to selective, potent enzymatic inhibition of PARP, several additional approaches may be employed to inhibit the cellular activity of PARP in cells or in experimental animals. The inhibition of intracellular calcium mobilization protects against oxidant-induced PARP activation, NAD+depletion, and cell necrosis, as demonstrated in thymocytes (Virag et al., 1999, Mol. Pharmacol., 56:824-833) and in intestinal epithelial cells (Karczewski et al., 1999, Biochem Pharmacol., 57:19-26). Similar to calcium chelators, intracellular zinc chelators have been shown to protect against oxidant-mediated PARP activation and cell necrosis (Virag et al., 1999, Br J Pharmacol., 126:769-777). Intracellular purines (inosine, hypoxanthine), in addition to a variety of effects, may also exert biological actions as inhibitors of PARP (Virag et al., 2001, FASEB J., 15:99-107).

Combination Therapy

In certain embodiments of the present invention, the methods of the invention further comprise treating cancer, specifically lung cancer, by administering to a subject a PARP inhibitor with at least one growth factor inhibitor in combination with another anti-cancer therapy including but not limited to surgery, radiation therapy (e.g., X ray), gene therapy, immunotherapy, DNA therapy, adjuvant therapy, neoadjuvant therapy, viral therapy, RNA therapy, or nanotherapy.

Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, by a significant period of time. The conjugate and the other pharmacologically active agent may be administered to a patient simultaneously, sequentially or in combination. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

In some embodiments, the dosage form is a kit, e.g., with packaging and, optionally, instructions for use. For example, the at least one PARP inhibitor and at least one growth factor inhibitor may be provided together (e.g., in a single vial or tablet), or separately (e.g., in vials ready for dissolution with or without additional inactive agents such as one or more pharmaceutically acceptable carrier, diluent or excipients.

### Radiation Therapy

Radiation therapy (or radiotherapy) is the medical use of ionizing radiation as part of cancer treatment to control malignant cells. Radiotherapy may be used for curative or adjuvant cancer treatment. It is used as palliative treatment (where cure is not possible and the aim is for local disease control or symptomatic relief) or as therapeutic treatment (where the therapy has survival benefit and it can be curative). Radiotherapy is used for the treatment of malignant tumors and may be used as the primary therapy. It is also common to combine radiotherapy with surgery, chemotherapy, hormone therapy or some mixture of the three. Most common cancer types can be treated with radiotherapy in some way. The precise treatment intent (curative, adjuvant, neoadjuvant, therapeutic, or palliative) will depend on the tumour type, location, and stage, as well as the general health of the patient.

Radiation therapy is commonly applied to the cancerous tumor. The radiation fields may also include the draining lymph nodes if they are clinically or radiologically involved with tumor, or if there is thought to be a risk of subclinical malignant spread. It is necessary to include a margin of normal tissue around the tumor to allow for uncertainties in daily set-up and internal tumor motion.

Radiation therapy works by damaging the DNA of cells. The damage is caused by a photon, electron, proton, neutron, or ion beam directly or indirectly ionizing the atoms which make up the DNA chain. Indirect ionization happens as a result of the ionization of water, forming free radicals, notably hydroxyl radicals, which then damage the DNA. In the most common forms of radiation therapy, most of the radiation effect is through free radicals. Because cells have mechanisms for repairing DNA damage, breaking the DNA on both strands proves to be the most significant technique in modifying cell characteristics. Because cancer cells generally are undifferentiated and stem cell-like, they reproduce more, and have a diminished ability to repair sub-lethal damage compared to most healthy differentiated cells. The DNA damage is inherited through cell division, accumulating damage to the cancer cells, causing them to die or reproduce more slowly. Proton radiotherapy works by sending protons with varying kinetic energy to precisely stop at the tumor.

Gamma rays are also used to treat some types of cancer including lung cancer. In the procedure called gamma-knife surgery, multiple concentrated beams of gamma rays are directed on the growth in order to kill the cancerous cells. The beams are aimed from different angles to focus the radiation on the growth while minimizing damage to the surrounding tissues.

Radiosensitizers are known to increase the sensitivity of cancerous cells to the toxic effects of electromagnetic radiation. Many cancer treatment protocols currently employ radiosensitizers activated by the electromagnetic radiation of x-rays. Examples of x-ray activated radiosensitizers include, but are not limited to, the following: metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5-iododeoxyuridine (IUdR), bromodeoxycytidine, fluorodeoxyuridine (FudR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, photoffin, benzoporphyrin derivatives, NPe6, tin etioporphyrin SnET2, pheoborbide-alpha, bacteriochlorophyll-alpha, naphthalocyanines, phthalocyanines, zinc phthalocyanine, and therapeutically effective analogs and derivatives of the same.

### Gene Therapy Agents

Gene therapy agents insert copies of genes into a specific set of a patient's cells, and can target both cancer and non-cancer cells. The goal of gene therapy can be to replace altered genes with functional genes, to stimulate a patient's immune response to cancer, to make cancer cells more sensitive to chemotherapy, to place “suicide” genes into cancer cells, or to inhibit angiogenesis. Genes may be delivered to target cells using viruses, liposomes, or other carriers or vectors. This may be done by injecting the gene-carrier composition into the patient directly, or ex vivo, with infected cells being introduced back into a patient. Such compositions are suitable for use in the present invention.

### Adjuvant Therapy

Adjuvant therapy is a treatment given after the primary treatment to increase the chances of a cure. Adjuvant therapy may include chemotherapy, radiation therapy, hormone therapy, or biological therapy.

Because the principal purpose of adjuvant therapy is to kill any cancer cells that may have spread, treatment is usually systemic (uses substances that travel through the bloodstream, reaching and affecting cancer cells all over the body). For example, adjuvant therapy for lung cancer involves chemotherapy or hormone therapy, either alone or in combination.

Adjuvant chemotherapy is the use of drugs to kill cancer cells. For example, research has shown that using chemotherapy as adjuvant therapy for early stage lung cancer helps to prevent the original cancer from returning. Adjuvant chemotherapy is usually a combination of anticancer drugs, which has been shown to be more effective than a single anticancer drug.

While not being bound by theory, adjuvant hormone therapy deprives cancer cells of the female hormone estrogen, for example, which some breast cancer cells need to grow. Most often, adjuvant hormone therapy is treatment with the drug tamoxifen. For example, research has shown that when tamoxifen is used as adjuvant therapy for early stage breast cancer, it helps to prevent the original cancer from returning and also helps to prevent the development of new cancers in the other breast.

The ovaries are the main source of estrogen prior to menopause. For premenopausal women with estrogen-sensitive cancer, adjuvant hormone therapy may involve tamoxifen to deprive the cancer cells of estrogen. Drugs to suppress the production of estrogen by the ovaries are under investigation. Alternatively, surgery may be performed to remove the ovaries.

Radiation therapy is sometimes used as a local adjuvant treatment. Radiation therapy is considered adjuvant treatment when it is given before or after surgical treatment, e.g., a mastectomy. Such treatment is intended to destroy cancer cells that have spread to nearby parts of the body, such as the chest wall or lymph nodes. In the case of breast-sparing surgery, radiation therapy is part of primary therapy, not adjuvant therapy.

### Neoadjuvant Therapy

Neoadjuvant therapy refers to a treatment given before the primary treatment. Examples of neoadjuvant therapy include chemotherapy, radiation therapy, and hormone therapy. In treating breast cancer, neoadjuvant therapy allows patients with large breast cancer to undergo breast-conserving surgery. In treating lung cancer, neoadjuvant therapy refers to a treatment given before the primary surgical treatment. Examples of neoadjuvant therapy include chemotherapy and radiation therapy.

### Oncolytic Viral Therapy

Viral therapy for cancer utilizes a type of viruses called oncolytic viruses. An oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site.

There are two main approaches for generating tumor selectivity: transductional and non-transductional targeting. Transductional targeting involves modifying the specificity of viral coat protein, thus increasing entry into target cells while reducing entry to non-target cells. Non-transductional targeting involves altering the genome of the virus so it can only replicate in cancer cells. This can be done by either transcription targeting, where genes essential for viral replication are placed under the control of a tumor-specific promoter, or by attenuation, which involves introducing deletions into the viral genome that eliminate functions that are dispensable in cancer cells, but not in normal cells. There are also other, slightly more obscure methods.

Chen et al., (2001) used CV706, a prostate-specific adenovirus, in conjunction with radiotherapy on prostate cancer in mice. The combined treatment results in a synergistic increase in cell death, as well as a significant increase in viral burst size (the number of virus particles released from each cell lysis).

ONYX-015 has undergone trials in conjunction with chemotherapy. The combined treatment gives a greater response than either treatment alone, but the results have not been entirely conclusive. ONYX-015 has shown promise in conjunction with radiotherapy.

Viral agents administered intravenously can be particularly effective against metastatic cancers, which are especially difficult to treat conventionally. However, bloodborne viruses can be deactivated by antibodies and cleared from the blood stream quickly e.g., by Kupffer cells (extremely active phagocytic cells in the liver, which are responsible for adenovirus clearance). Avoidance of the immune system until the tumour is destroyed could be the biggest obstacle to the success of oncolytic virus therapy. To date, no technique used to evade the immune system is entirely satisfactory. It is in conjunction with conventional cancer therapies that oncolytic viruses show the most promise, since combined therapies operate synergistically with no apparent negative effects.

The specificity and flexibility of oncolytic viruses means they have the potential to treat a wide range of cancers including lung cancer with minimal side effects. Oncolytic viruses have the potential to solve the problem of selectively killing cancer cells.

### Nanotherapy

Nanometer-sized particles have novel optical, electronic, and structural properties that are not available from either individual molecules or bulk solids. When linked with tumor-targeting moieties, such as tumor-specific ligands or monoclonal antibodies, these nanoparticles can be used to target cancer-specific receptors, tumor antigens (biomarkers), and tumor vasculatures with high affinity and precision. The formulation and manufacturing process for cancer nanotherapy is disclosed in patent U.S. Pat. No. 7,179,484, and article M. N. Khalid, P. Simard, D. Hoarau, A. Dragomir, J. Leroux, Long Circulating Poly(Ethylene Glycol) Decorated Lipid Nanocapsules Deliver Docetaxel to Solid Tumors, Pharmaceutical Research, 23(4), 2006, all of which are herein incorporated by reference in their entireties.

### RNA Therapy

RNA including but not limited to siRNA, shRNA, microRNA may be used to modulate gene expression and treat cancers. Double stranded oligonucleotides are formed by the assembly of two distinct oligonucleotide sequences where the oligonucleotide sequence of one strand is complementary to the oligonucleotide sequence of the second strand; such double stranded oligonucleotides are generally assembled from two separate oligonucleotides (e.g., siRNA), or from a single molecule that folds on itself to form a double stranded structure (e.g., shRNA or short hairpin RNA). These double stranded oligonucleotides known in the art all have a common feature in that each strand of the duplex has a distinct nucleotide sequence, wherein only one nucleotide sequence region (guide sequence or the antisense sequence) has complementarity to a target nucleic acid sequence and the other strand (sense sequence) comprises nucleotide sequence that is homologous to the target nucleic acid sequence.

MicroRNAs (miRNA) are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression.

Certain RNA inhibiting agents may be utilized to inhibit the expression or translation of messenger RNA (“mRNA”) that is associated with a cancer phenotype. Examples of such agents suitable for use herein include, but are not limited to, short interfering RNA (“siRNA”), ribozymes, and antisense oligonucleotides. Specific examples of RNA inhibiting agents suitable for use herein include, but are not limited to, Candy, Sirna-027, fomivirsen, and angiozyme.

### Small Molecule Enzymatic Inhibitors

Certain small molecule therapeutic agents are able to target the tyrosine kinase enzymatic activity or downstream signal transduction signals of certain cell receptors such as epidermal growth factor receptor (“EGFR”) or vascular endothelial growth factor receptor (“VEGFR”). Such targeting by small molecule therapeutics can result in anti-cancer effects. Examples of such agents suitable for use herein include, but are not limited to, imatinib, gefitinib, erlotinib, lapatinib, canertinib, ZD6474, sorafenib (BAY 43-9006), ERB-569, and their analogues and derivatives, as well as additional growth factor inhibitors disclosed herein.

### Anti-Metastatic Agents

The process whereby cancer cells spread from the site of the original tumor to other locations around the body is termed cancer metastasis. Certain agents have anti-metastatic properties, designed to inhibit the spread of cancer cells. Examples of such agents suitable for use herein include, but are not limited to, marimastat, bevacizumab, trastuzumab, rituximab, erlotinib, MMI-166, GRN163L, hunter-killer peptides, tissue inhibitors of metalloproteinases (TIMPs), their analogues, derivatives and variants.

### Chemopreventative Agents

Certain pharmaceutical agents can be used to prevent initial occurrences of cancer, or to prevent recurrence or metastasis. Administration with such chemopreventative agents in combination with eflornithine-NSAID conjugates of the invention can act to both treat and prevent the recurrence of cancer. Examples of chemopreventative agents suitable for use herein include, but are not limited to, tamoxifen, raloxifene, tibolone, bisphosphonate, ibandronate, estrogen receptor modulators, aromatase inhibitors (letrozole, anastrozole), luteinizing hormone-releasing hormone agonists, goserelin, vitamin A, retinal, retinoic acid, fenretinide, 9-cis-retinoid acid, 13-cis-retinoid acid, all-trans-retinoic acid, isotretinoin, tretinoid, vitamin B6, vitamin B12, vitamin C, vitamin D, vitamin E, cyclooxygenase inhibitors, non-steroidal anti-inflammatory drugs (NSAIDs), aspirin, ibuprofen, celecoxib, polyphenols, polyphenol E, green tea extract, folic acid, glucaric acid, interferon-alpha, anethole dithiol ethione, zinc, pyridoxine, finasteride, doxazosin, selenium, indole-3-carbinal, alpha-difluoromethylornithine, carotenoids, beta-carotene, lycopene, antioxidants, coenzyme Q10, flavonoids, quercetin, curcumin, catechins, epigallocatechin gallate, N-acetylcysteine, indole-3-carbinol, inositol hexaphosphate, isoflavones, glucanic acid, rosemary, soy, saw palmetto, and calcium. An additional example of chemopreventative agents suitable for use in the present invention is cancer vaccines. These can be created through immunizing a patient with all or part of a cancer cell type that is targeted by the vaccination process.

In some embodiments, the therapeutic agents for the treatment include antibodies or reagents that bind to PARP, and thereby lower the level of PARP in a subject. In other embodiments, cellular expression can be modulated in order to affect the level of PARP and/or PARP activity in a subject. Therapeutic and/or prophylactic polynucleotide molecules can be delivered using gene transfer and gene therapy technologies. Still other agents include small molecules that bind to or interact with the PARP and thereby affect the function thereof, and small molecules that bind to or interact with nucleic acid sequences encoding PARP, and thereby affect the level of PARP. These agents may be administered alone or in combination with other types of treatments known and available to those skilled in the art for treating diseases. In some embodiment, the PARP inhibitors for the treatment can be used either therapeutically, prophylactically, or both. The PARP inhibitors may either directly act on PARP or modulate other cellular constituents which then have an effect on the level of PARP. In some embodiments, the PARP inhibitors inhibit the activity of PARP.

Clinical Efficacy:

### Classification of Lung Cancer

Lung cancer is currently the most frequently diagnosed major cancer and the most common cause of cancer mortality in males worldwide. The main types of lung cancer are small cell lung carcinoma and non-small cell lung carcinoma. This distinction is important because the treatment varies. Non-small cell lung carcinoma (NSCLC) is sometimes treated with surgery, while small cell lung carcinoma (SCLC) usually responds better to chemotherapy and radiation (Vaporciyan, A A. et al. 2000 Cancer Medicine. B C Decker. pp. 1227-1292), en.wikipedia.org/wiki/Lung_cancer-cite_note-Cancer_Medicine-3#cite_note-Cancer_Medicine-3 Non-small cell lung cancer (NSCLC) consists mainly of squamous-cell carcinoma, adenocarcinoma, and large-cell carcinoma. NSCLC represents around 80% of all lung cancers (Bunn P A and Thatcher N, The Oncologist 2008; 13(suppl 1): 1-4). The World Health Organization/International Association for the Study of Lung Cancer Histological Classification of Lung and Pleural Tumours is disclosed in Table 1 in Brambilla E. et al. The new World Health Organization classification of lung tumours, Eur Respir J2001; 18:1059-1068, which is herein incorporated by reference in its entirety.

Lung tumours are divided into two broad categories of small cell carcinoma (SCLC 20-25% of cases) and non-small cell lung cancer (NSCLC 70-80% of cases) based on clinical behaviour and histological appearance. Other rarer tumour types include carcinoids (typical or atypical), carcinosarcomas, pulmonary blastomas, giant and spindle cell carcinomas. NSCLC is further divided histologically into three main disease subtypes of: squamous cell carcinoma, adenocarcinoma and large cell carcinoma.

Lung tumors are classified primarily on their cytological origin. The relative frequency of subtypes varies in different geographical regions and the figures cited therefore represent broad approximations. Clinically, the most important division is between SCLC and NSCLC. Small cell tumours generally metastasize early in the course of the disease, but are relatively responsive to chemotherapeutic drugs: they are therefore managed in a different way to non-small cell lesions.

Lung cancers are not one disease and generally represent heterogeneous tumors, consisting of cells with different histological and genetic subtypes. This intra-tumour heterogeneity of lung cancer has led to the conclusion that lung carcinomas arise from a multipotent stem cell-like (or stem cell) component of the bronchial epithelium.

SCLC is seldom surgically resectable, usually widespread at presentation and is generally both more chemosensitive and radiosensitive.

NSCLC: Treatment is based on the stage of the disease at presentation (which may be assessed by, for example, thoracic CT, PET scan, brain MRI). Stage I-II are usually resected and locally advanced stages (III) are often treated by combined modality treatments (e.g., neoadjuvant chemotherapy, resection if stage IIIA or radiotherapy). If overt distant metastases are detected, therapy is often palliative and chemotherapy has been shown to improve median survival and quality of life.

For example, see Pass H. I., et al. (2000). Lung Cancer: Principles and Practice. Lippincott Williams & Wilkins, Philadelphia (pubs), pp. 453-517; Dongiovanni D., et al. (Feb. 1, 2008). “Gefitinib (ZD1839): Therapy in selected patients with non-small cell lung cancer (NSCLC)?” Lung Cancer; Pao W. et al. (2004). “EGF receptor gene mutations are common in lung cancers from “never smokers” and are associated with sensitivity of tumors to gefitinib and erlotinib,” Proc Nall Acad Sci USA 101:13306-11. PMID 15329413 (Full text); Sordella R. et al. (2004). “Gefitinib-sensitizing EGFR mutations in lung cancer activate anti-apoptotic pathways,” Science 305:1163-7. PMID 15284455; Rossi S. ed. (2004). Australian Medicines Handbook 2004. Adelaide: Australian Medicines Handbook. ISBN 0-9578521-4-2; Sasaki H. et al. (December 2007). “EGFR exon 20 insertion mutation in Japanese lung cancer,” Lung Cancer 58(3):324-8; Thurlbeck W M and Churg A M (eds) (1995). Pathology of the Lung: second edition. Thieme Medical Publishers, Inc. NY; pp. 437-551; Garber M. E. et al. (2001). “Diversity of gene expression in adenocarcinoma of the lung,” Proc Nall Acad Sci USA 98:13784-13789. Medline 11707590; Bhattacharjee A. et al. (2001). “Classification of human lung carcinomas by mRNA expression profiling reveals distinct adenocarcinoma subclasses,” Proc Natl Acad Sci USA 98:13790-513795. Medline 11707567; Heighway J. et al. (2002). “Expression profiling of primary non-small cell lung cancer for target identification,” Oncogene 21:7749-7763. Medline 12400018; Kiyohara C. et al. (2002). “Genetic polymorphisms and lung cancer susceptibility: a review,” Lung Cancer 37:241-256. Medline 12234692; Hwang S. J., et al. (2003). “Lung cancer risk in germline p53 mutation carriers: association between an inherited cancer predisposition, cigarette smoking, and cancer risk,” Hum Genet. 2003; 113:238-243. Medline 12802680; Borczuk A. C. et al. (2003). “Non-small-cell lung cancer molecular signatures recapitulate lung developmental pathways,” Am J Pathol 163:1949-1960. Medline 14578194; Mitsuuchi Y. et al. (2002). “Cytogenetics and molecular genetics of lung cancer,” Am J Med Genet. 115:183-188. Medline 12407699; Zabarovsky E. R. et al. (2002). “Tumor suppressor genes on chromosome 3p involved in the pathogenesis of lung and other cancers,” Oncogene 21:6915-6935. Medline 12362274.

SCLC: If the tumour is confined to one hemithorax, a combined modality therapy chemo- and radiotherapy is indicated: in more advanced disease (overt distant metastases in brain, liver, bones, surrenal glands or other organs) chemotherapy will be palliative though an excellent remission might be obtained in more than half of the patients.

### Staging of Lung Cancer:

The stages represent the nature and extent of spread of a neoplasm and, thus, the therapeutic options and prognosis in individual patients. Stages also provide a standard by which various therapies can be compared. A combination of clinical, laboratory, radiologic, and pathologic investigations are used to stage various neoplasms.

Different staging systems have been developed. The most widely used scheme for staging NSCLC is the TNM classification. The UICC and the AJCC initially introduced this system in 1972 with staging and end-results reporting. This scheme has been modified and refined over the years. The TNM classification in the publications of the UICC and the AJCC are identical. They are formulated together but appear in separate books—namely, the UICC TNM Classification of Malignant Tumors and the AJCC Cancer Staging Manual. TNM is a dual system with a pretreatment Clinical classification (cTNM or TNM) and a postsurgical histopathologic Pathological classification (pTNM). Both classifications are retained unaltered in the patient's record. The former is used for the choice of treatment; the latter is used for the estimation of prognosis and the possible selection of adjuvant therapy.

The TNM staging system takes into account the degree of spread of the primary tumor, represented by T; the extent of regional lymph node involvement, represented by N; and the presence or absence of distant metastases, represented by M. The TNM system is used for all lung carcinomas except SCLCs, which are staged separately. In the TNM systems, 4 stages are further subdivided into I-III and A or B subtypes. These stages have important therapeutic and prognostic implications.

### Non-Small Cell Lung Cancer T Stages

Tis: Cancer is found only in the layer of cells lining the air passages. It has not invaded other lung tissues. This stage is also known as carcinoma in situ.

T1: The cancer is no larger than 3 centimeters (slightly less than 1¼ inches), has not spread to the visceral pleura (membranes that surround the lungs) and does not affect the main branches of the bronchi.

T2: The cancer has one or more of the following features: it is larger than 3 cm, it involves a main bronchus, but is not closer than 2 cm (about ¾ inch) to the point where the trachea (windpipe) branches into the left and right main bronchi; it has spread to the visceral pleura; the cancer may partially clog the airways, but this has not caused the entire lung to collapse or develop pneumonia.

T3: The cancer has one or more of the following features: it has spread to the chest wall, the diaphragm (breathing muscle that separates the chest from the abdomen), the mediastinal pleura (membranes surrounding the space between the two lungs), or parietal pericardium (membranes of the sac surrounding the heart). It involves a main bronchus and is closer than 2 cm (about ¾ inch) to the point where the trachea (windpipe) branches into the left and right main bronchi, but does not involve this area. It has grown into the airways enough to cause one lung to entirely collapse or to cause pneumonia of the entire lung.

T4: The cancer has one or more of the following features: it has spread to the mediastinum (space behind the chest bone and in front of the heart), the heart, the trachea (windpipe), the esophagus (tube connecting the throat to the stomach), the backbone or the point where the windpipe branches into the left and right main bronchi. Two or more separate tumor nodules are present in the same lobe. There is a malignant pleural effusion (fluid containing cancer cells in the space surrounding the lung).

Non-Small Cell Lung Cancer N Stages

N0: No spread to lymph nodes

N1: Spread to lymph nodes within the lung, hilar lymph nodes (located around the area where the bronchus enters the lung). Metastases affect lymph nodes only on the same sides as the cancerous lung.

N2: Spread to lymph nodes around the point where the windpipe branches into the left and right bronchi or to lymph nodes in the medistinum (space behind the chest bone and in front of the heart). Affected lymph nodes are on the same side of the cancerous lung.

N3: Spread to lymph nodes near the collarbone on either side, to hilar or mediastinal lymph nodes on the side opposite the cancerous lung.

Non-Small Cell Lung Cancer M Stages

M0: No distant spread

M1: Distant spread is present. Sites considered distant include other lobes of the lungs, lymph nodes further than those mentioned in N stages, and other organs or tissues such as the liver, bones, or brain.

### Staging of Small Cell Lung Cancer

For small cell lung cancers, a two-stage system is most often used. These are “limited stage” and “extensive stage”. Limited stage usually means that the cancer is only in one lung and in lymph nodes on the same side of the chest.

Spread of the cancer to the other lung, to lymph nodes on the other side of the chest, or to distant organs indicates extensive disease. Many doctors consider small cell lung cancer that has spread to the fluid around the lung to be an extensive stage.

Small cell lung cancer is staged in this way because it helps separate tumors that can be treated more effectively with radiation therapy from those which cannot. About two-thirds of the people with small cell lung cancer will have extensive disease when their cancer is first found.

Clinical efficacy may be measured by any method known in the art. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD≧6 months. The CBR for the combination therapy of a PARP inhibitor with a growth factor inhibitor such as EGFR inhibitor may be compared to that of the monotherapy with the growth factor inhibitor such as, for example, IRESSA alone. In some embodiments, CBR of the combination therapy is at least about 60%. In some embodiments, CBR is at least about 30%, at least about 40%, or at least about 50%. In some embodiments, the CBR is about 60% or higher. In some embodiments, the therapeutic effect includes reduction in size of a lung tumor, reduction in metastasis, complete remission, partial remission, stable disease, or a pathologic complete response.

### Additional Characterization of Lung Cancer

In some embodiments disclosed herein, the methods include pre-determining that a cancer is treatable by PARP modulators. Some such methods comprise identifying a level of PARP in a lung cancer sample of a patient, determining whether the level of PARP expression in the sample is greater than a pre-determined value, and, if the PARP expression is greater than said predetermined value, treating the patient with a combination of a taxane (e.g., paclitaxel), a platinum complex (e.g., carboplatin) and a PARP inhibitor such as BA. In other embodiments, the methods comprise identifying a level of PARP in a non-small cell lung cancer sample of a patient, determining whether the level of PARP expression in the sample is greater than a pre-determined value, and, if the PARP expression is greater than said predetermined value, treating the patient with a PARP inhibitor such as BA. In other embodiments, the methods include pre-determining that a cancer is treatable by PARP modulators. Some such methods comprise identifying a level of PARP in a lung cancer sample of a patient, determining whether the level of PARP expression in the sample is greater than a pre-determined value, and, if the PARP expression is greater than said predetermined value, treating the patient with a combination of a growth factor inhibitor, such as an EGFR inhibitor e.g., IRESSA, and a PARP inhibitor such as BA.

Numerous cancers have been found in BRCA1 and BRCA2 mutation carriers including lung cancer, breast cancer, leukemia, brain cancer, skin cancer, lymphoma, and colon cancer. These tumor cells have lost a specific mechanism that repair damaged DNA. BRCA1 plays a significant role in non-small cell lung cancer (NSCLC). Not only can it be used to predict outcome for patients with NSCLC, but it may also prove to be a valuable tool in choosing the best therapy for them (Rosell R, et al. PLoS ONE. 2007; 2(11): el 129). BRCA1 and BRCA2 are important for DNA double-strand break repair by homologous recombination, and mutations in these genes predispose to breast and other cancers. PARP is involved in base excision repair, a pathway in the repair of DNA single-strand breaks. BRCA1 or BRCA2 dysfunction sensitizes cells to the inhibition of PARP enzymatic activity, resulting in chromosomal instability, cell cycle arrest and subsequent apoptosis (Jones C, Plummer E R. PARP inhibitors and cancer therapy—early results and potential applications. Br J Radial. 2008 October; 81 Spec No 1:S2-5; Drew Y, Calvert H. The potential of PARP inhibitors in genetic breast and ovarian cancers. Ann N Y Acad. Sci. 2008 September; 1138:136-45; Farmer H, et.al. Targeting the DNA repair defect in BRCA mutant cells as a therapeutic strategy. Nature. 2005 Apr. 14; 434(7035):917-21).

Patients deficient in BRCA genes can have up-regulated levels of PARP. PARP up-regulation may be an indicator of defective DNA-repair pathways and unrecognized BRCA-like genetic defects. Assessment of PARP gene expression and impaired DNA repair especially defective homologous recombination DNA repair can be used as an indicator of tumor sensitivity to PARP inhibitor. Hence, in some embodiments, treatment of lung cancer can be enhanced by identifying early onset of cancer in BRCA and homologous recombination DNA repair deficient patients by measuring the level of PARP. The BRCA and homologous recombination DNA repair deficient patients treatable by PARP inhibitors can be identified if PARP is up-regulated. Further, such homologous recombination DNA repair deficient patients can be treated with PARP inhibitors.

In some embodiments, a sample is collected from a patient having a lung lesion or growth suspected of being cancerous. While such sample may be any available biological tissue, in most cases the sample will be a portion of the suspected lung lesion, whether obtained by minimally invasive biopsy or by therapeutic surgery. Such sample may also include all or part of one or more lymph nodes extracted during the therapeutic surgery. PARP expression may then be analyzed. In some embodiments, if the PARP expression is above a predetermined level (e.g., is up-regulated vis-à-vis normal tissue) the patient may be treated with a PARP inhibitor in combination with an antimetabolite and a platinum agent. In other embodiments, if the PARP expression is above a predetermined level (e.g., is up-regulated vis-à-vis normal tissue) the patient may be treated with a PARP inhibitor, including a PARP inhibitor, such as BA, in combination with a growth factor inhibitor, such as EGFR inhibitor. It is thus to be understood that, while some embodiments described herein are directed to treatment of lung cancer, in some embodiments the lung cancer need not have these characteristics so long as the threshold PARP up-regulation is satisfied.

In some embodiments, tumors that are homologous recombination deficient are identified by evaluating levels of PARP expression. If up-regulation of PARP is observed, such tumors can be treated with PARP inhibitors and growth factor inhibitors. Another embodiment is a method for treating a homologous recombination deficient cancer comprising evaluating level of PARP expression and, if overexpression is observed, the cancer is treated with a PARP inhibitor and a growth factor inhibitor.

Screening patients for various factors before treatment may allow predictions about the clinical benefit of the treatment. For example, DNA in pleural effusion fluid can be used to detect EGFR mutations (Kimura H, Fujiwara Y, Sone T et al. Br J Cancer 2006; 95:1390-1395). Another potential independent prognostic factor in patients with NSCLC is glucose metabolic activity, which closely reflects response to gefitinib therapy. As such, fluorodeoxyglucose (FDG)-positron emission tomography, an imaging method that uses the higher glycolytic rate of tumor cells, may be a valuable clinical tool; increased FDG uptake is an independent prognostic factor in patients with International Union against Cancer stage I/II NSCLC, and less distinctively so for stage III tumors (Su H, Bodenstein C, Dumont R A et al. Clin Cancer Res 2006; 12:5659-5667; Eschmann S M, Friedel G, Paulsen F et al. Eur J Nucl Med Mol Imaging 2006; 33:263-269).

### Sample Collection, Preparation and Separation

Biological samples may be collected from a variety of sources from a patient including a body fluid sample, or a tissue sample. Samples collected can be human normal and tumor samples, nipple aspirants. The samples can be collected from individuals repeatedly over a longitudinal period of time (e.g., about once a day, once a week, once a month, biannually or annually). Obtaining numerous samples from an individual over a period of time can be used to verify results from earlier detections and/or to identify an alteration in biological pattern as a result of, for example, disease progression, drug treatment, etc.

Sample preparation and separation can involve any of the procedures, depending on the type of sample collected and/or analysis of PARP. Such procedures include, by way of example only, concentration, dilution, adjustment of pH, removal of high abundance polypeptides (e.g., albumin, gamma globulin, and transferin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transportions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC) etc.

Identifying the Level of PARP

The poly (ADP-ribose) polymerase (PARP) is also known as poly (ADP-ribose) synthase and poly ADP-ribosyltransferase. PARP catalyzes the formation of mono- and poly (ADP-ribose) polymers which can attach to cellular proteins (as well as to itself) and thereby modify the activities of those proteins. The enzyme plays a role in regulation of transcription, cell proliferation, and chromatin remodeling (for review see: D. D'amours et al. “Poly (ADP-ribosylation reactions in the regulation of nuclear functions,” Biochem. J. 342: 249-268 (1999)).

PARP comprises an N-terminal DNA binding domain, an automodification domain and a C-terminal catalytic domain and various cellular proteins interact with PARP. The N-terminal DNA binding domain contains two zinc finger motifs. Transcription enhancer factor-1 (TEF-1), retinoid X receptor α, DNA polymerase α, X-ray repair cross-complementing factor-1 (XRCC1) and PARP itself interact with PARP in this domain. The automodification domain contains a BRCT motif, one of the protein-protein-interaction modules. This motif is originally found in the C-terminus of BRCA1 (breast cancer susceptibility protein 1) and is present in various proteins related to DNA repair, recombination and cell-cycle checkpoint control. POU-homeodomain-containing octamer transcription factor-1 (Oct-1), Yin Yang (YY)1 and ubiquitin-conjugating enzyme 9 (ubc9) could interact with this BRCT motif in PARP.

More than 15 members of the PARP family of genes are present in the mammalian genome. PARP family proteins and poly(ADP-ribose) glycohydrolase (PARG), which degrades poly(ADP-ribose) to ADP-ribose, could be involved in a variety of cell regulatory functions including DNA damage response and transcriptional regulation and may be related to carcinogenesis and the biology of cancer in many respects.

Several PARP family proteins have been identified. Tankyrase has been found as an interacting protein of telomere regulatory factor 1 (TRF-1) and is involved in telomere regulation. Vault PARP (VPARP) is a component in the vault complex, which acts as a nuclear-cytoplasmic transporter. PARP-2, PARP-3 and 2,3,7,8-tetrachlorodibenzo-p-dioxin inducible PARP (TiPARP) have also been identified. Therefore, poly (ADP-ribose) metabolism could be related to a variety of cell regulatory functions.

A member of this gene family is PARP-1. The PARP-1 gene product is expressed at high levels in the nuclei of cells and is dependent upon DNA damage for activation. Without being bound by any theory, it is believed that PARP-1 binds to DNA single or double stranded breaks through an amino terminal DNA binding domain. The binding activates the carboxy terminal catalytic domain and results in the formation of polymers of ADP-ribose on target molecules. PARP-1 is itself a target of poly ADP-ribosylation by virtue of a centrally located automodification domain. The ribosylation of PARP-1 causes dissociation of the PARP-1 molecules from the DNA. The entire process of binding, ribosylation, and dissociation occurs very rapidly. It has been suggested that this transient binding of PARP-1 to sites of DNA damage results in the recruitment of DNA repair machinery or may act to suppress the recombination long enough for the recruitment of repair machinery.

The source of ADP-ribose for the PARP reaction is nicotinamide adenosine dinucleotide (NAD). NAD is synthesized in cells from cellular ATP stores and thus high levels of activation of PARP activity can rapidly lead to depletion of cellular energy stores. It has been demonstrated that induction of PARP activity can lead to cell death that is correlated with depletion of cellular NAD and ATP pools. PARP activity is induced in many instances of oxidative stress or during inflammation. For example, during reperfusion of ischemic tissues reactive nitric oxide is generated and nitric oxide results in the generation of additional reactive oxygen species including hydrogen peroxide, peroxynitrate and hydroxyl radical. These latter species can directly damage DNA and the resulting damage induces activation of PARP activity. Frequently, it appears that sufficient activation of PARP activity occurs such that the cellular energy stores are depleted and the cell dies. A similar mechanism is believed to operate during inflammation when endothelial cells and pro-inflammatory cells synthesize nitric oxide which results in oxidative DNA damage in surrounding cells and the subsequent activation of PARP activity. The cell death that results from PARP activation is believed to be a major contributing factor in the extent of tissue damage that results from ischemia-reperfusion injury or from inflammation.

In some embodiments, the level of PARP in a sample from a patient is compared to predetermined standard sample. The sample from the patient is typically from a diseased tissue, such as cancer cells or tissues. The standard sample can be from the same patient or from a different subject. The standard sample is typically a normal, non-diseased sample. However, in some embodiments, such as for staging of disease or for evaluating the efficacy of treatment, the standard sample is from a diseased tissue. The standard sample can be a combination of samples from several different subjects. In some embodiments, the level of PARP from a patient is compared to a pre-determined level. This pre-determined level is typically obtained from normal samples. As described herein, a “pre-determined PARP level” may be a level of PARP used to, by way of example only, evaluate a patient that may be selected for treatment, evaluate a response to a PARP inhibitor treatment, evaluate a response to a combination of a PARP inhibitor and a second therapeutic agent treatment, and/or diagnose a patient for cancer, inflammation, pain and/or related conditions. A pre-determined PARP level may be determined in populations of patients with or without cancer. The pre-determined PARP level can be a single number, equally applicable to every patient, or the pre-determined PARP level can vary according to specific subpopulations of patients. For example, men might have a different pre-determined PARP level than women; non-smokers may have a different pre-determined PARP level than smokers. Age, weight, and height of a patient may affect the pre-determined PARP level of the individual. Furthermore, the pre-determined PARP level can be a level determined for each patient individually. The pre-determined PARP level can be any suitable standard. For example, the pre-determined PARP level can be obtained from the same or a different human for whom a patient selection is being assessed. In one embodiment, the pre-determined PARP level can be obtained from a previous assessment of the same patient. In such a manner, the progress of the selection of the patient can be monitored over time. In addition, the standard can be obtained from an assessment of another human or multiple humans, e.g., selected groups of humans. In such a manner, the extent of the selection of the human for whom selection is being assessed can be compared to suitable other humans, e.g., other humans who are in a similar situation to the human of interest, such as those suffering from similar or the same condition(s).

In some embodiments of the present invention the change of PARP from the pre-determined level is about 0.5 fold, about 1.0 fold, about 1.5 fold, about 2.0 fold, about 2.5 fold, about 3.0 fold, about 3.5 fold, about 4.0 fold, about 4.5 fold, or about 5.0 fold. In some embodiments is fold change is less than about 1, less than about 5, less than about 10, less than about 20, less than about 30, less than about 40, or less than about 50. In other embodiments, the changes in PARP level compared to a predetermined level is more than about 1, more than about 5, more than about 10, more than about 20, more than about 30, more than about 40, or more than about 50. Preferred fold changes from a pre-determined level are about 0.5, about 1.0, about 1.5, about 2.0, about 2.5, and about 3.0.

The analysis of PARP levels in patients is particularly valuable and informative, as it allows the physician to more effectively select the best treatments, as well as to utilize more aggressive treatments and therapy regimens based on the up-regulated or down-regulated level of PARP. More aggressive treatment, or combination treatments and regimens, can serve to counteract poor patient prognosis and overall survival time. Armed with this information, the medical practitioner can choose to provide certain types of treatment such as treatment with PARP inhibitors, and/or more aggressive therapy.

In monitoring a patient's PARP levels, over a period of time, which may be days, weeks, months, and in some cases, years, or various intervals thereof, the patient's body fluid sample, e.g., serum or plasma, can be collected at intervals, as determined by the practitioner, such as a physician or clinician, to determine the levels of PARP, and compared to the levels in normal individuals over the course or treatment or disease. For example, patient samples can be taken and monitored every month, every two months, or combinations of one, two, or three month intervals according to the invention. In addition, the PARP levels of the patient obtained over time can be conveniently compared with each other, as well as with the PARP values, of normal controls, during the monitoring period, thereby providing the patient's own PARP values, as an internal, or personal, control for long-term PARP monitoring.

### Techniques for Analysis of PARP

The analysis of the PARP may include analysis of PARP gene expression, including an analysis of DNA, RNA, analysis of the level of PARP and/or analysis of the activity of PARP including a level of mono- and poly-ADP-ribozylation. Without limiting the scope of the present invention, any number of techniques known in the art can be employed for the analysis of PARP and they are all within the scope of the present invention. Some of the examples of such detection technique are given below but these examples are in no way limiting to the various detection techniques that can be used in the present invention.

Gene Expression Profiling: Methods of gene expression profiling include methods based on hybridization analysis of polynucleotides, polyribonucleotides methods based on sequencing of polynucleotides, polyribonucleotides and proteomics-based methods. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS), Comparative Genome Hybridisation (CGH), Chromatin Immunoprecipitation (ChIP), Single nucleotide polymorphism (SNP) and SNP arrays, Fluorescent in situ Hybridization (FISH), Protein binding arrays and DNA microarray (also commonly known as gene or genome chip, DNA chip, or gene array), RNAmicroarrays.

Reverse Transcriptase PCR (RT-PCR): One of the most sensitive and most flexible quantitative PCR-based gene expression profiling methods is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. For example, the starting material can be typically total RNA isolated from human tumors or tumor cell lines, and corresponding normal tissues or cell lines, respectively. Thus RNA can be isolated from a variety of normal and diseased cells and tissues, for example tumors, including breast, lung, colorectal, prostate, brain, liver, kidney, pancreas, spleen, thymus, testis, ovary, uterus, etc., or tumor cell lines. If the source of mRNA is a primary tumor, mRNA can be extracted, for example, from frozen or archived fixed tissues, for example paraffin-embedded and fixed (e.g., formalin-fixed) tissue samples. General methods for mRNA extraction are well known in the art and are disclosed in standard textbooks of molecular biology, including Ausubel et al., Current Protocols of Molecular Biology, John Wiley and Sons (1997).

In particular, RNA isolation can be performed using purification kit, buffer set and protease from commercial manufacturers, according to the manufacturer's instructions. RNA prepared from tumor can be isolated, for example, by cesium chloride density gradient centrifugation. As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. The derived cDNA can then be used as a template in the subsequent PCR reaction.

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe. Real time PCR is compatible both with quantitative competitive PCR, where internal competitor for each target sequence is used for normalization, and with quantitative comparative PCR using a normalization gene contained within the sample, or a housekeeping gene for RT-PCR.

Fluorescence Microscopy: Some embodiments of the invention include fluorescence microscopy for analysis of PARP. Fluorescence microscopy enables the molecular composition of the structures being observed to be identified through the use of fluorescently-labeled probes of high chemical specificity such as antibodies. It can be done by directly conjugating a fluorophore to a protein and introducing this back into a cell. Fluorescent analogue may behave like the native protein and can therefore serve to reveal the distribution and behavior of this protein in the cell. Along with NMR, infrared spectroscopy, circular dichroism and other techniques, protein intrinsic fluorescence decay and its associated observation of fluorescence anisotropy, collisional quenching and resonance energy transfer are techniques for protein detection. The naturally fluorescent proteins can be used as fluorescent probes. The jellyfish aequorea victoria produces a naturally fluorescent protein known as green fluorescent protein (GFP). The fusion of these fluorescent probes to a target protein enables visualization by fluorescence microscopy and quantification by flow cytometry.

By way of example only, some of the probes are labels such as, fluorescein and its derivatives, carboxyfluoresceins, rhodamines and their derivatives, atto labels, fluorescent red and fluorescent orange: cy3/cy5 alternatives, lanthanide complexes with long lifetimes, long wavelength labels—up to 800 nm, DY cyanine labels, and phycobili proteins. By way of example only, some of the probes are conjugates such as, isothiocyanate conjugates, streptavidin conjugates, and biotin conjugates. By way of example only, some of the probes are enzyme substrates such as, fluorogenic and chromogenic substrates. By way of example only, some of the probes are fluorochromes such as, FITC (green fluorescence, excitation/emission=506/529 nm), rhodamine B (orange fluorescence, excitation/emission=560/584 nm), and nile blue A (red fluorescence, excitation/emission=636/686 nm). Fluorescent nanoparticles can be used for various types of immunoassays. Fluorescent nanoparticles are based on different materials, such as, polyacrylonitrile, and polystyrene etc. Fluorescent molecular rotors are sensors of micro environmental restriction that become fluorescent when their rotation is constrained. Few examples of molecular constraint include increased dye (aggregation), binding to antibodies, or being trapped in the polymerization of actin. IEF (isoelectric focusing) is an analytical tool for the separation of ampholytes, mainly proteins. An advantage for IEF-gel electrophoresis with fluorescent IEF-marker is the possibility to directly observe the formation of gradient. Fluorescent IEF-marker can also be detected by UV-absorption at 280 nm (20° C.).

A peptide library can be synthesized on solid supports and, by using coloring receptors, subsequent dyed solid supports can be selected one by one. If receptors cannot indicate any color, their binding antibodies can be dyed. The method can not only be used on protein receptors, but also on screening binding ligands of synthesized artificial receptors and screening new metal binding ligands as well. Automated methods for HTS and FACS (fluorescence activated cell sorter) can also be used. A FACS machine originally runs cells through a capillary tube and separate cells by detecting their fluorescent intensities.

Immunoassays: Some embodiments of the invention include immunoassay for the analysis of PARP. In immunoblotting like the western blot of electrophoretically separated proteins a single protein can be identified by its antibody. Immunoassay can be competitive binding immunoassay where analyte competes with a labeled antigen for a limited pool of antibody molecules (e.g., radioimmunoassay, EMIT). Immunoassay can be non-competitive where antibody is present in excess and is labeled. As analyte antigen complex is increased, the amount of labeled antibody-antigen complex may also increase (e.g., ELISA). Antibodies can be polyclonal if produced by antigen injection into an experimental animal, or monoclonal if produced by cell fusion and cell culture techniques. In immunoassay, the antibody may serve as a specific reagent for the analyte antigen.

Without limiting the scope and content of the present invention, some of the types of immunoassays are, by way of example only, RIAs (radioimmunoassay), enzyme immunoassays like ELISA (enzyme-linked immunosorbent assay), EMIT (enzyme multiplied immunoassay technique), microparticle enzyme immunoassay (MEIA), LIA (luminescent immunoassay), and FIA (fluorescent immunoassay). These techniques can be used to detect biological substances in the nasal specimen. The antibodies—either used as primary or secondary ones—can be labeled with radioisotopes (e.g., 125I), fluorescent dyes (e.g., FITC) or enzymes (e.g., HRP or AP) which may catalyse fluorogenic or luminogenic reactions.

Biotin, or vitamin H is a co-enzyme which inherits a specific affinity towards avidin and streptavidin. This interaction makes biotinylated peptides a useful tool in various biotechnology assays for quality and quantity testing. To improve biotin/streptavidin recognition by minimizing steric hindrances, it can be necessary to enlarge the distance between biotin and the peptide itself. This can be achieved by coupling a spacer molecule (e.g., 6-nitrohexanoic acid) between biotin and the peptide.

The biotin quantitation assay for biotinylated proteins provides a sensitive fluorometric assay for accurately determining the number of biotin labels on a protein. Biotinylated peptides are widely used in a variety of biomedical screening systems requiring immobilization of at least one of the interaction partners onto streptavidin coated beads, membranes, glass slides or microtiter plates. The assay is based on the displacement of a ligand tagged with a quencher dye from the biotin binding sites of a reagent. To expose any biotin groups in a multiply labeled protein that are sterically restricted and inaccessible to the reagent, the protein can be treated with protease for digesting the protein.

EMIT is a competitive binding immunoassay that avoids the usual separation step. A type of immunoassay in which the protein is labeled with an enzyme, and the enzyme-protein-antibody complex is enzymatically inactive, allowing quantitation of unlabelled protein. Some embodiments of the invention include ELISA to analyze PARP. ELISA is based on selective antibodies attached to solid supports combined with enzyme reactions to produce systems capable of detecting low levels of proteins. It is also known as enzyme immunoassay or EIA. The protein is detected by antibodies that have been made against it, that is, for which it is the antigen. Monoclonal antibodies are often used.

The test may require the antibodies to be fixed to a solid surface, such as the inner surface of a test tube, and a preparation of the same antibodies coupled to an enzyme. The enzyme may be one (e.g., β-galactosidase) that produces a colored product from a colorless substrate. The test, for example, may be performed by filling the tube with the antigen solution (e.g., protein) to be assayed. Any antigen molecule present may bind to the immobilized antibody molecules. The antibody-enzyme conjugate may be added to the reaction mixture. The antibody part of the conjugate binds to any antigen molecules that are bound previously, creating an antibody-antigen-antibody “sandwich”. After washing away any unbound conjugate, the substrate solution may be added. After a set interval, the reaction is stopped (e.g., by adding 1 N NaOH) and the concentration of colored product formed is measured in a spectrophotometer. The intensity of color is proportional to the concentration of bound antigen.

ELISA can also be adapted to measure the concentration of antibodies, in which case, the wells are coated with the appropriate antigen. The solution (e.g., serum) containing antibody may be added. After it has had time to bind to the immobilized antigen, an enzyme-conjugated anti-immunoglobulin may be added, consisting of an antibody against the antibodies being tested for. After washing away unreacted reagent, the substrate may be added. The intensity of the color produced is proportional to the amount of enzyme-labeled antibodies bound (and thus to the concentration of the antibodies being assayed).

Some embodiments of the invention include radioimmunoassays to analyze PARP. Radioactive isotopes can be used to study in vivo metabolism, distribution, and binding of small amount of compounds. Radioactive isotopes of ¹H, ¹²C, ³¹P, ³²S, and ¹²⁷I in body are used such as ³H, ¹⁴C, ³²P, ³⁵S, and ¹²⁵I. In receptor fixation method in 96 well plates, receptors may be fixed in each well by using antibody or chemical methods and radioactive labeled ligands may be added to each well to induce binding. Unbound ligands may be washed out and then the standard can be determined by quantitative analysis of radioactivity of bound ligands or that of washed-out ligands. Then, addition of screening target compounds may induce competitive binding reaction with receptors. If the compounds show higher affinity to receptors than standard radioactive ligands, most of radioactive ligands would not bind to receptors and may be left in solution. Therefore, by analyzing quantity of bound radioactive ligands (or washed-out ligands), testing compounds' affinity to receptors can be indicated.

The filter membrane method may be needed when receptors cannot be fixed to 96 well plates or when ligand binding needs to be done in solution phase. In other words, after ligand-receptor binding reaction in solution, if the reaction solution is filtered through nitrocellulose filter paper, small molecules including ligands may go through it and only protein receptors may be left on the paper. Only ligands that strongly bound to receptors may stay on the filter paper and the relative affinity of added compounds can be identified by quantitative analysis of the standard radioactive ligands.

Some embodiments of the invention include fluorescence immunoassays for the analysis of PARP. Fluorescence based immunological methods are based upon the competitive binding of labeled ligands versus unlabeled ones on highly specific receptor sites. The fluorescence technique can be used for immunoassays based on changes in fluorescence lifetime with changing analyte concentration. This technique may work with short lifetime dyes like fluorescein isothiocyanate (FITC) (the donor) whose fluorescence may be quenched by energy transfer to eosin (the acceptor). A number of photoluminescent compounds may be used, such as cyanines, oxazines, thiazines, porphyrins, phthalocyanines, fluorescent infrared-emitting polynuclear aromatic hydrocarbons, phycobiliproteins, squaraines and organo-metallic complexes, hydrocarbons and azo dyes.

Fluorescence based immunological methods can be, for example, heterogenous or homogenous. Heterogenous immunoassays comprise physical separation of bound from free labeled analyte. The analyte or antibody may be attached to a solid surface. The technique can be competitive (for a higher selectivity) or noncompetitive (for a higher sensitivity). Detection can be direct (only one type of antibody used) or indirect (a second type of antibody is used). Homogenous immunoassays comprise no physical separation. Double-antibody fluorophore-labeled antigen participates in an equilibrium reaction with antibodies directed against both the antigen and the fluorophore. Labeled and unlabeled antigen may compete for a limited number of anti-antigen antibodies.

Some of the fluorescence immunoassay methods include simple fluorescence labeling method, fluorescence resonance energy transfer (FRET), time resolved fluorescence (TRF), and scanning probe microscopy (SPM). The simple fluorescence labeling method can be used for receptor-ligand binding, enzymatic activity by using pertinent fluorescence, and as a fluorescent indicator of various in vivo physiological changes such as pH, ion concentration, and electric pressure. TRF is a method that selectively measures fluorescence of the lanthanide series after the emission of other fluorescent molecules is finished. TRF can be used with FRET and the lanthanide series can become donors or acceptors. In scanning probe microscopy, in the capture phase, for example, at least one monoclonal antibody is adhered to a solid phase and a scanning probe microscope is utilized to detect antigen/antibody complexes which may be present on the surface of the solid phase. The use of scanning tunneling microscopy eliminates the need for labels which normally is utilized in many immunoassay systems to detect antigen/antibody complexes.

Protein identification methods: By way of example only, protein identification methods include low-throughput sequencing through Edman degradation, mass spectrometry techniques, peptide mass fingerprinting, de novo sequencing, and antibody-based assays. The protein quantification assays include fluorescent dye gel staining, tagging or chemical modification methods (i.e., isotope-coded affinity tags (ICATS), combined fractional diagonal chromatography (COFRADIC)). The purified protein may also be used for determination of three-dimensional crystal structure, which can be used for modeling intermolecular interactions. Common methods for determining three-dimensional crystal structure include x-ray crystallography and NMR spectroscopy. Characteristics indicative of the three-dimensional structure of proteins can be probed with mass spectrometry. By using chemical crosslinking to couple parts of the protein that are close in space, but far apart in sequence, information about the overall structure can be inferred. By following the exchange of amide protons with deuterium from the solvent, it is possible to probe the solvent accessibility of various parts of the protein.

In one embodiment, fluorescence-activated cell-sorting (FACS) is used to identify PARP expressing cells. FACS is a specialized type of flow cytometry. It provides a method for sorting a heterogenous mixture of biological cells into two or more containers, one cell at a time, based upon the specific light scattering and fluorescent characteristics of each cell. It provides quantitative recording of fluorescent signals from individual cells as well as physical separation of cells of particular interest. In yet another embodiment, microfluidic based devices are used to evaluate PARP expression.

Mass spectrometry can also be used to characterize PARP from patient samples. The two methods for ionization of whole proteins are electrospray ionization (ESI) and matrix-assisted laser desorption/ionization (MALDI). In the first, intact proteins are ionized by either of the two techniques described above, and then introduced to a mass analyser. In the second, proteins are enzymatically digested into smaller peptides using an agent such as trypsin or pepsin. Other proteolytic digest agents are also used. The collection of peptide products are then introduced to the mass analyser. This is often referred to as the “bottom-up” approach of protein analysis.

Whole protein mass analysis is conducted using either time-of-flight (TOF) MS, or Fourier transform ion cyclotron resonance (FT-ICR). The instrument used for peptide mass analysis is the quadrupole ion trap. Multiple stage quadrupole-time-of-flight and MALDI time-of-flight instruments also find use in this application.

Two methods used to fractionate proteins, or their peptide products from an enzymatic digestion. The first method fractionates whole proteins and is called two-dimensional gel electrophoresis. The second method, high performance liquid chromatography is used to fractionate peptides after enzymatic digestion. In some situations, it may be necessary to combine both of these techniques.

There are two ways mass spectroscopy can be used to identify proteins. Peptide mass uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein is present in the original sample.

Tandem MS is also a method for identifying proteins. Collision-induced dissociation is used in mainstream applications to generate a set of fragments from a specific peptide ion. The fragmentation process primarily gives rise to cleavage products that break along peptide bonds.

A number of different algorithmic approaches have been described to identify peptides and proteins from tandem mass spectrometry (MS/MS), peptide de novo sequencing and sequence tag based searching. One option that combines a comprehensive range of data analysis features is PEAKS. Other existing mass spec analysis software include: Peptide fragment fingerprinting SEQUEST, Mascot, OMSSA and X!Tandem).

Proteins can also be quantified by mass spectrometry. Typically, stable (e.g., non-radioactive) heavier isotopes of carbon (C13) or nitrogen (N15) are incorporated into one sample while the other one is labeled with corresponding light isotopes (e.g., C¹² and N¹⁴). The two samples are mixed before the analysis. Peptides derived from the different samples can be distinguished due to their mass difference. The ratio of their peak intensities corresponds to the relative abundance ratio of the peptides (and proteins). The methods for isotope labeling are SILAC (stable isotope labeling with amino acids in cell culture), trypsin-catalyzed O18 labeling, ICAT (isotope coded affinity tagging), ITRAQ (isotope tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument.

N-terminal sequencing aids in the identification of unknown proteins, confirm recombinant protein identity and fidelity (reading frame, translation start point, etc.), aid the interpretation of NMR and crystallographic data, demonstrate degrees of identity between proteins, or provide data for the design of synthetic peptides for antibody generation, etc. N-terminal sequencing utilizes the Edman degradative chemistry, sequentially removing amino acid residues from the N-terminus of the protein and identifying them by reverse-phase HPLC. Sensitivity can be at the level of 100s femtomoles and long sequence reads (20-40 residues) can often be obtained from a few 10s picomoles of starting material. Pure proteins (>90%) can generate easily interpreted data, but insufficiently purified protein mixtures may also provide useful data, subject to rigorous data interpretation. N-terminally modified (especially acetylated) proteins cannot be sequenced directly, as the absence of a free primary amino-group prevents the Edman chemistry. However, limited proteolysis of the blocked protein (e.g., using cyanogen bromide) may allow a mixture of amino acids to be generated in each cycle of the instrument, which can be subjected to database analysis in order to interpret meaningful sequence information. C-terminal sequencing is a post-translational modification, affecting the structure and activity of a protein. Various disease situations can be associated with impaired protein processing and C-terminal sequencing provides an additional tool for the investigation of protein structure and processing mechanisms.

### Formulations and Administration

In some embodiments are provided pharmaceutical compositions of at least one PARP inhibitor and at least one growth factor inhibitor, or a pharmaceutically acceptable salt, isomer, solvate or tautomer of thereof. In some embodiment, the pharmaceutical formulation includes one or more pharmaceutically acceptable carrier, diluent or excipient. For example, such as those described herein. In some embodiments, the PARP inhibitor is 4-iodo-3-nitrobenzamide, or a pharmaceutical acceptable salt, isomer, solvate or tautomer thereof. In some embodiments, the one or more pharmaceutically acceptable carrier, diluent or excipient acts as a solubilizer to increase the solubility of the one or more PARP inhibitor and/or the one or more growth factor inhibitor in the pharmaceutical composition, versus the same compounds in water.

The present invention also relates to pharmaceutical compositions comprising an aromatic nitrobenzamide compound or its metabolites in combination with a growth factor inhibitor and a solubilizer wherein the solubilizer comprises an oligosaccharide. A preferred embodiment of an oligosaccharide is a cyclic oligosaccharide, such as cyclodextrin. More specifically, the invention relates to pharmaceutical compositions comprising the nitro compound 4-iodo-3-nitrobenzamide or a salt, solvate, isomer, tautomer, metabolite, analog, or prodrug thereof and a cyclodextrin.

The present invention also relates to pharmaceutical compositions comprising an aromatic nitrobenzamide compound or its metabolites in combination with a growth factor inhibitor and a solubilizer, where the solubilizer comprises a surfactant. More specifically, it relates to pharmaceutical compositions comprising the nitro compound 4-iodo-3-nitrobenzamide or a salt, solvate, isomer, tautomer, metabolite, analog, or prodrug thereof and a surfactant having enhanced solubility.

The present invention also relates to pharmaceutical compositions comprising an aromatic nitrobenzamide compound or its metabolites in combination with a growth factor inhibitor and a solubilizer where the solubilizer comprises a co-solvent. More specifically, it relates to pharmaceutical compositions comprising the nitro compound 4-iodo-3-nitrobenzamide or a salt, solvate, isomer, tautomer, metabolite, analog, or prodrug thereof and a co-solvent having enhanced solubility.

The present invention also relates to pharmaceutical compositions comprising an aromatic nitrobenzamide compound or its metabolites in combination with a growth factor inhibitor and a mixture of (1) a cylodextrin and a surfactant, (2) a cyclodextrin and a co-solvent, (3) a surfactant and a co-solvent, or (4) a cyclodextrin, a surfactant, and a co-solvent having enhanced solubility. More specifically, it relates to pharmaceutical compositions comprising the nitro compound 4-iodo-3-nitrobenzamide or a salt, solvate, isomer, tautomer, metabolite, analog, or prodrug thereof and a mixture of (1) a cylodextrin and a surfactant, (2) a cyclodextrin and a co-solvent, (3) a surfactant and a co-solvent, or (4) a cyclodextrin, a surfactant, and a co-solvent having enhanced solubility. A preferred formulation is with 25% beta-cyclodextrin (e.g. hydroxypropyl-beta-cyclodextrin) and 10 mM phosphate at pH 7.4. The formulations of PARP inhibitors for treating cancer have been disclosed in U.S. patent application Ser. No. 12/015,403 (Publication No. US 2008-0176946 A1), which is herein incorporated by reference in its entirety.

The methods of treatment as disclosed herein can be e.g.; via oral administration, transmucosal administration, buccal administration, nasal administration, inhalation, parental administration, intravenous, subcutaneous, intramuscular, sublingual, transdermal administration, ocular administration, and rectal administration.

Pharmaceutical compositions of PARP inhibitors suitable for use in treatment following the identification of a disease treatable by PARP inhibitors in a subject, include compositions wherein the active ingredient is contained in a therapeutically or prophylactically effective amount, i.e., in an amount effective to achieve therapeutic or prophylactic benefit. The actual amount effective for a particular application will depend, inter alia, on the condition being treated and the route of administration. Determination of an effective amount is well within the capabilities of those skilled in the art. The pharmaceutical compositions comprise the PARP inhibitors, one or more pharmaceutically acceptable carriers, diluents or excipients, and optionally additional therapeutic agents, for example, at least one growth factor inhibitor and, optionally, additional therapeutic agents. The compositions can be formulated for sustained or delayed release.

The compositions can be administered by injection, topically, orally, transdermally, rectally, or via inhalation. The oral form in which the therapeutic agent is administered can include powder, tablet, capsule, solution, or emulsion. The effective amount can be administered in a single dose or in a series of doses separated by appropriate time intervals, such as hours. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Suitable techniques for preparing pharmaceutical compositions of the therapeutic agents are well known in the art.

A preferred dose of 4-iodo-3-nitrobenzamide (BA) is 4 mg/kg IV over one hour, twice weekly, beginning on day 1 (doses of BA are preferably separated by at least 2 days). BA treatment is preferably given twice weekly as an IV infusion for three consecutive weeks in each 28-day cycle. Other preferred doses include 0.5, 1.0, 1.4, 2.8 and 4 mg/kg either as a monotherapy or a combination therapy.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular PARP inhibitor, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

### EXAMPLES

### Example 1

### Combination of BA with EGFR Inhibitor

### Cell Culture

Lung adenocarcinoma HCC827 cells are cultured in Dulbecco Modified Eagle Medium with 10% fetal bovine serum. HCC827 cells contain the E746_A750del mutation of EGFR and are examined for the effects of gefitinib (IRESSA) in combination with a PARP inhibitor, 4-iodo-3-nitrobenzamide (BA), on the growth of HCC827 cells. Cells are plated at a seeding density 10⁵ per P100 or at 10⁴ per P60 in growth media and incubated 12-18 h at 37° C., 5% CO₂. BA and the EGFR inhibitor, IRESSA, are added as a single dose for 72 hours. DMSO is used as a control. Cells are irradiated with 3Gy and 5Gy gamma-irradiation using γ-Irradiator Gammacell 40 Exactor (MDS Nordion, Canada). Following treatment, cells are analyzed with BrdU ELISA assay (Roche Applied Science), FACS based cell cycle assay or TUNEL.

### Compounds

BA is dissolved directly from dry powder in DMSO (cat #472301, Sigma-Aldrich) for each separate experiment, then the entire volume of the stock solution is used to prepare 111 nM, 313 nM and 1 uM working concentrations in cell culture medium to avoid any possibility of precipitation and the corresponding loss of compound. Control experiments are carried out with the matching volume/concentration of the vehicle (DMSO); in these controls, the cells show no changes in their growth or cell cycle distribution.

### PI Exclusion, Cell Cycle and TUNEL Assays (FACS)

After the addition of drugs, irradiation and incubation, cells are taken for counting and PI (Propidium Iodide) exclusion assay. One part of the cells is centrifuged and resuspended in 0.5 ml ice-cold PBS containing 5 μg/ml of PI. The other part of the cells is fixed in ice-cold 70% ethanol and stored in a freezer overnight. For cell cycle analysis, cells are stained with propidium iodide (PI) as described hereinabove. Cellular DNA content is determined by flow cytometry using BD LSRII FACS, and the percentages of cells in G1, S or G2/M are determined using ModFit software.

To detect apoptosis, the cells are labeled with the “In Situ Cell Death Detection Kit, Fluorescein” (Roche Diagnostics Corporation, Roche Applied Science, Indianapolis, Ind.). Briefly, fixed cells are centrifuged and washed once in phosphate-buffered saline (PBS) containing 1% bovine serum albumin (BSA), then resuspended in 2 ml permeabilization buffer (0.1% Triton X-100 and 0.1% sodium citrate in PBS) for 25 min at room temperature and washed twice in 0.2 ml PBS/1% BSA. The cells are resuspended in 50 μl TUNEL reaction mixture (TdT enzyme and labeling solution) and incubated for 60 min at 37° C. in a humidified dark atmosphere in an incubator. The labeled cells are washed once in PBS/1% BSA, then resuspended in 0.5 ml ice-cold PBS containing 1 μg/ml 4′,6-diamidino-2-phenylindole (DAPI) for at least 30 min. All cell samples are analyzed with a BD LSR II (BD Biosciences, San Jose, Calif.). All flow cytometry analyses are carried out using triplicate samples containing at least 30,000 cells each (typical results of independent experiments are shown). The coefficient of variance in all the experiments is equal or less than 0.01.

### Bromodeoxyuridine (BrdU) Labeling Assay and FACS-Based Cell Cycle Analysis

50 μl of BrdU (Sigma Chemical Co., St. Louis, Mo.) stock solution (1 mM) is added to achieve final concentration of 10 μM BrdU. Then cells are incubated for 30 min at 37° C. and fixed in ice-cold 70% ethanol and stored at 4° C. overnight. Fixed cells are centrifuged and washed once in 2 ml PBS, then resuspended in 0.7 ml of denaturation solution (0.2 mg/ml pepsin in 2 N HCl) for 15 min at 37° C. in the dark, then 1.04 ml 1M Tris buffer (Trizma base, Sigma Chemical Co.) is added to terminate the hydrolysis. Cells are washed in 2 ml PBS and resuspended in 100-μl (1:100 dilution) of anti-BrdU antibody (DakoCytomation, Carpinteria, Calif.) in TBFP permeable buffer (0.5% Tween-20, 1% bovine serum albumin and 1% fetal bovine serum in PBS), incubated for 25 min at room temperature in the dark and washed in 2 ml PBS. The primary antibody-labeled cells are resuspended in 100 μl ALEXA FLUOR® F(ab′)2 fragment of goat anti-mouse IgG (H+L) (1:200 dilution, 2 mg/mL, Molecular Probes, Eugene, Oreg.) in TBFP buffer and incubated for 25 min at room temperature in the dark and washed in 2 ml PBS, then resuspended in 0.5 ml ice-cold PBS containing 1 μg/ml 4′,6-diamidino-2-phenylindole (DAPI) for at least 30 min. All cell samples are analyzed with a BD LSR II (BD Biosciences, San Jose, Calif.). All flow cytometry analyses are carried out using triplicate samples containing at least 30,000 cells each (typical results of independent experiments are shown). The coefficient of variance in all the experiments is equal or less than 0.01.

### Results

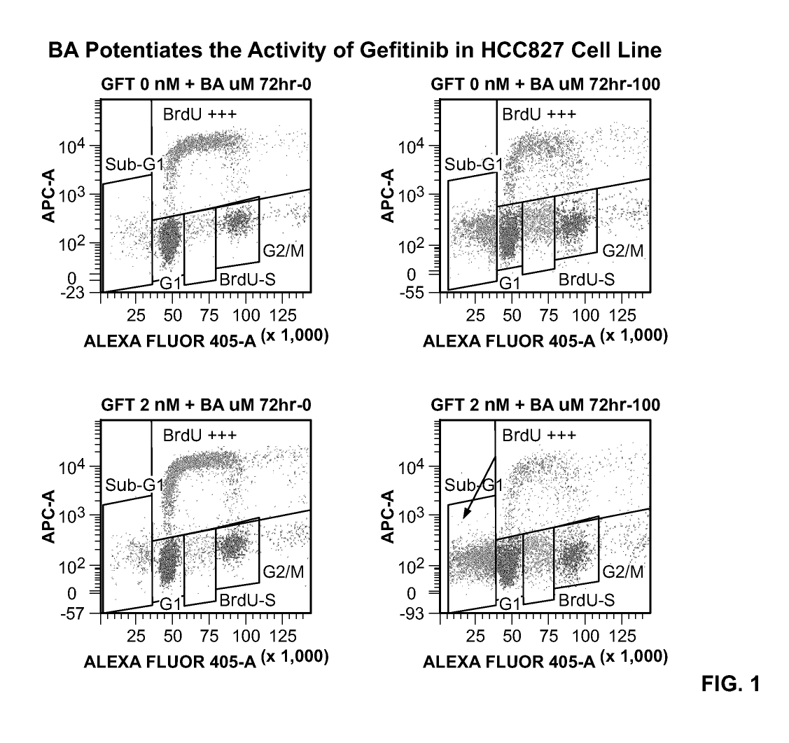

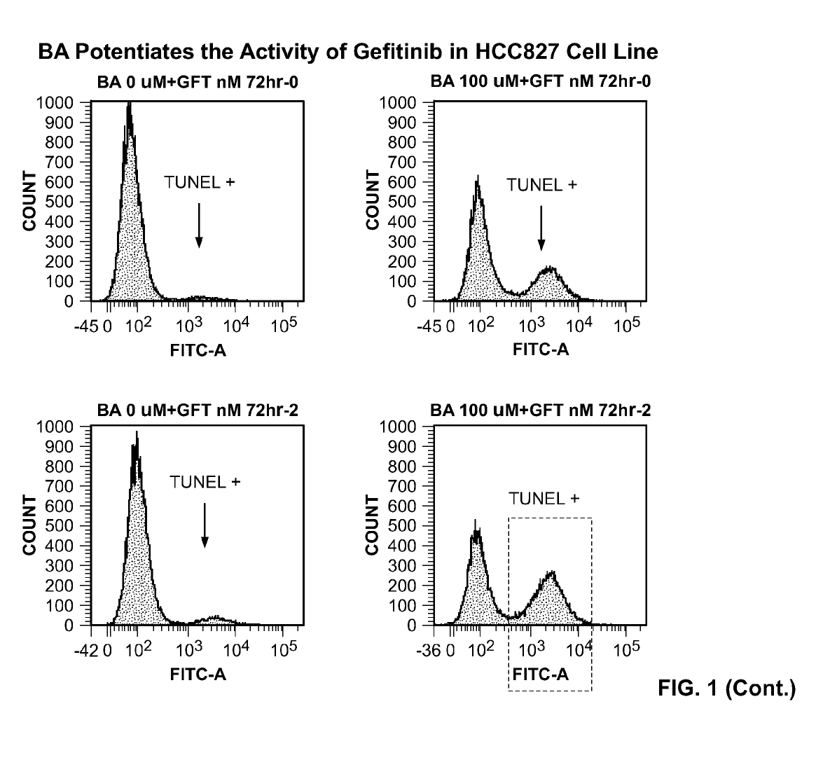

The HCC827 non-small cell lung cancer (NSCLC) cell line is a well characterized model for analysis of EGFR inhibitors. As shown in FIG. 1, BA potentiates the activity of the EGFR inhibitor, IRESSA, in the HCC827 cell line. The response of lung cancer cells HCC827 to the combination of BA with IRESSA is summarized in Table 1.

TABLE 1
Summary of the response of lung cancer cells HCC827 to the
combinations of BA with IRESSA (gefitinib)

| | G1 | S | G2/M | Viable Cell | Sub-G1 | TUNEL (+) |
|---|---|---|---|---|---|---|
| GFT 0 nM + | | | | | | |
| BA uM | | | | | | |
| 0 | 65.9 | 24.3 | 6.3 | 100.0 | 1.9 | 3.2 |
| 50 | 46.3 | 40.6 | 8.8 | 65.0 | 2.4 | 5.7 |
| 100 | 43.8 | 19.9 | 12.5 | 25.0 | 15.8 | 32.8 |
| GFT 2 nM + | | | | | | |
| BA uM | | | | | | |
| 0 | 51.0 | 34.4 | 9.2 | 56.0 | 3.4 | 6.2 |
| 50 | 52.3 | 28.9 | 9.4 | 37.0 | 6.6 | 13.3 |
| 100 | 38.9 | 12.6 | 12.3 | 15.0 | 27.4 | 46.5 |

### Example 1A

### Combination of BA with EGFR Inhibitor

The effects of the BA and its nitroso metabolite (BNO) on cell proliferation and the cell cycle of the HCC827 NSLC tumor cell line in combination with gefitinib were investigated.

BA and BNO were tested in the presence of the gefitinib (LC Laboratories G-4408, BGF-103) as shown in the schedule indicated in the Table 2.

TABLE 2

| Part | Agent or agents | BA | BNO | Cell line |
|---|---|---|---|---|
| 1. | EGFR inhibitor (Gefitinib) | +/− | +/− | HCC827 Non-Small Cell |
| | | | | Lung Carcinoma |

First, the IC50 for the EGFR inhibitor was determined for the HCC827 cell line.

Second, two concentrations of BA (100 μM and 50 μM) and BNO (25 μM and 50 μM) were tested in combination with gefitinib. The gefitinib in this experiment was tested in the concentrations corresponding IC50 for the HCC827 cell line. The compounds were simultaneously added to the cells for 72 hours.

The two lowest active doses of BA and BNO in combination gefitinib were tested for their effects on the cell cycle and cell death. This analysis was carried out by FACS assay based on DNA content, BrdU incorporation and Terminal deoxynucleotidyl transferase dUTP nick end labeling (TUNEL).

### Material and Methods

Cell Culture

HCC827 non-small cell lung carcinoma cells were obtained from ATCC, Rockville, Md. HCC827 non-small cell lung carcinoma cells were cultured in Roswell Park Memorial Institute 1640 culture medium (RPMI1640) with 10% fetal calf serum (FC2). Cells were plated at 2×10⁵ per P100 or at 10⁴ per P60 (for assays requiring up to 3 days of culture), in the presence of different concentrations of BA and BNO, gefitinib or DMSO (vehicle control) or prior the addition of the BA/BNO were irradiated with 3Gy and 5Gy gamma-irradiation using Gammacell 40 Exactor (MDS Nordion, Canada). Central dose rate of approximately 1.30 Gy/minute (130 rad/minute). Each of the two special form Cesium sources has a nominal activity of 66.6 TBq (1800 Ci). Together they produce a central dose rate of 1.30 Gy/minute (130 rad/minute)±15% in the sample container. Typical dose uniformity is ±7% over a 260 mm (10.2 in.) diameter and a 100 mm (3.9 in) height.

Following treatment, the number of vital cells was measured. Percentage of the vital (proliferating) cells under different treatments was calculated relative to the control (untreated) cells.

Immunofluorescence

For the immunostaining cells were grown to 40% confluency and treated with different concentrations and combinations of the compounds as described above on chambered cover slides. Cells were washed with PBS and extracted with 0.5% Triton X-100 in PHEM buffer (60 mM PIPES, 25 mM HEPES, 10 mM EGTA, and 4 mM MgSO₄) for 5 mM at 37° C. Cells were then fixed with 4% paraformaldehyde in PHEM buffer for 20 min at 37° C., washed three times with 0.2% Triton X-100 in PBS, and incubated with blocking solution containing 3% bovine serum albumin in PHEM buffer for 1 h. Cells were incubated with primary antibodies in 3% bovine serum albumin in PHEM buffer for 1 h, washed three times with PBS plus 0.2% Triton X-100, and further incubated with fluorescent secondary antibodies (Molecular Probes, Inc.) at 1:500 dilution. DNA was stained with 4′,6-diamidino-2-phenylindole (DAPI). Cells were washed three times with PBS, mounted, and viewed with a 63× objective on a Zeiss LSM 510 META confocal microscope. Images were acquired with a CCD camera using the LSM 510 META Detector, and LSM 510 software and further processed with Adobe Photoshop.

### Compounds

BA and BNO were dissolved directly from dry powder to 20 mM stock solution in DMSO (cat #472301, Sigma-Aldrich) for each separate experiment, then the entire volume of the stock solution was used to prepare 10, 50 and 100 μM working concentrations in cell culture medium to avoid any possibility of precipitation and the corresponding loss of compound. Control experiments were carried out with the matching volume/concentration of the vehicle (DMSO); in these controls, the cells showed no changes in their growth or cell cycle distribution.

PI Exclusion and Cell Cycle Assays (FACS)

After the addition of the compounds as described above, irradiation and incubation, cells were trypsinized and aliquots of the samples were taken for counting and PI (Propidium Iodide) exclusion assay. One part of the cells was centrifuged and resuspended in 0.5 ml ice-cold PBS containing 5 ug/ml of PI. The other part of the cells was fixed in ice-cold 70% ethanol and stored in a freezer overnight. For cell cycle analysis, cells were stained with propidium iodide (PI) by standard procedures. Cellular DNA content was determined by flow cytometry using BD LSRII FACS, and the percentages of cells in G1, S or G2/M were determined using ModFit software.

All cell samples were analyzed as described in [5,6] using a BD LSR II or BD Aria (BD Biosciences, San Jose, Calif.). All flow cytometry analyses were carried out using triplicate samples containing at least 30,000 cells each and the final percentages of the cell cycle distribution were normalized to singlets viable cell population, excluding cell debris and polyploid cells according established procedure [4,5]. Typical results of 3 or more independent experiments are shown in Table 3. The coefficient of variance in all the experiments was equal or less than 0.01.

Bromodeoxyuridine (BrdU) Labeling Assay, TUNEL, and FACS Analysis

50 μl of BrdU (Sigma Chemical Co., St. Louis, Mo.) stock solution (1 mM) was added to achieve final concentration of 10 μM BrdU. Then cells were incubated for 30 min at 37° C. and fixed in ice-cold 70% ethanol and stored at 4° C. overnight. Fixed cells were centrifuged and washed once in 2 ml PBS, then resuspended in 0.7 ml of denaturation solution (0.2 mg/ml pepsin in 2 N HCl) for 15 min at 37° C. in the dark, then 1.04 ml 1M Tris buffer (Trizma base, Sigma Chemical Co.) was added to terminate the hydrolysis. Cells were washed in 2 ml PBS and resuspended in 100-μ1(1:100 dilution) of anti-BrdU antibody (DakoCytomation, Carpinteria, Calif.) in TBFP permeable buffer (0.5% Tween-20, 1% bovine serum albumin and 1% fetal bovine serum in PBS), incubated for 25 min at room temperature in the dark and washed in 2 ml PBS. The primary antibody-labeled cells were resuspended in 100 μl ALEXA FLUOR® F(ab)₂, fragment of goat anti-mouse IgG (H+L) (1:200 dilution, 2 mg/mL, Molecular Probes, Eugene, Oreg.) in TBFP buffer and incubated for 25 min at room temperature in the dark and washed in 2 ml PBS, then resuspended in 0.5 ml ice-cold PBS containing 1 μg/ml 4′,6-diamidino-2-phenylindole (DAPI) for at least 30 min [3, 6].

The cells were labeled for apoptosis detection with the “In Situ Cell Death Detection Kit, Fluorescein” (Roche Diagnostics Corporation, Roche Applied Science, Indianapolis, Ind.) and analyzed based on the modified protocol from Dr. Darzynkiewicz laboratory [4, 5]. Briefly, fixed cells were centrifuged and washed once in phosphate-buffered saline (PBS) containing 1% bovine serum albumin (BSA), then resuspended in 2 ml permeabilization buffer (0.1% Triton X-100 and 0.1% sodium citrate in PBS) for 25 min at room temperature and washed twice in 0.2 ml PBS/1% BSA. The cells were resuspended in 50 μl TUNEL reaction mixture (TdT enzyme and labeling solution) and incubated for 60 min at 37° C. in a humidified dark atmosphere in an incubator. The labeled cells were washed once in PBS/1% BSA, then resuspended in 0.5 ml ice-cold PBS containing 1 μg/ml 4′,6-diamidino-2-phenylindole (DAPI) for at least 30 min. All cell samples were analyzed with a BD LSR II (BD Biosciences, San Jose, Calif.) and the final percentages of the cell cycle distribution were normalized to singlets viable cell population, excluding cell debris and polyploid cells according established procedure [4, 5]. All flow cytometry analyses were carried out using triplicate samples containing at least 30,000 cells each (typical results of 3 or more independent experiments are shown). The coefficient of variance in all the experiments was equal or less than 0.01.

See also Roche Cell Proliferation ELISA, BrdU (chemiluminescence) Instruction Manual (Cat. No. 1 669, 915, version 4, August 2003).

### Results

HCC827 non-small cell lung carcinoma cells were tested for suitability for the FACS analysis based on DNA content and BrdU assay. The cell line was found suitable for this analysis.

Two different concentrations of each of BA and BNO were selected based on preliminary results of the proliferation and survival analysis. The concentrations of BA and BNO producing detectable effect were identified to be used in following analyses in combination with two doses of gefitinib (Table 2).

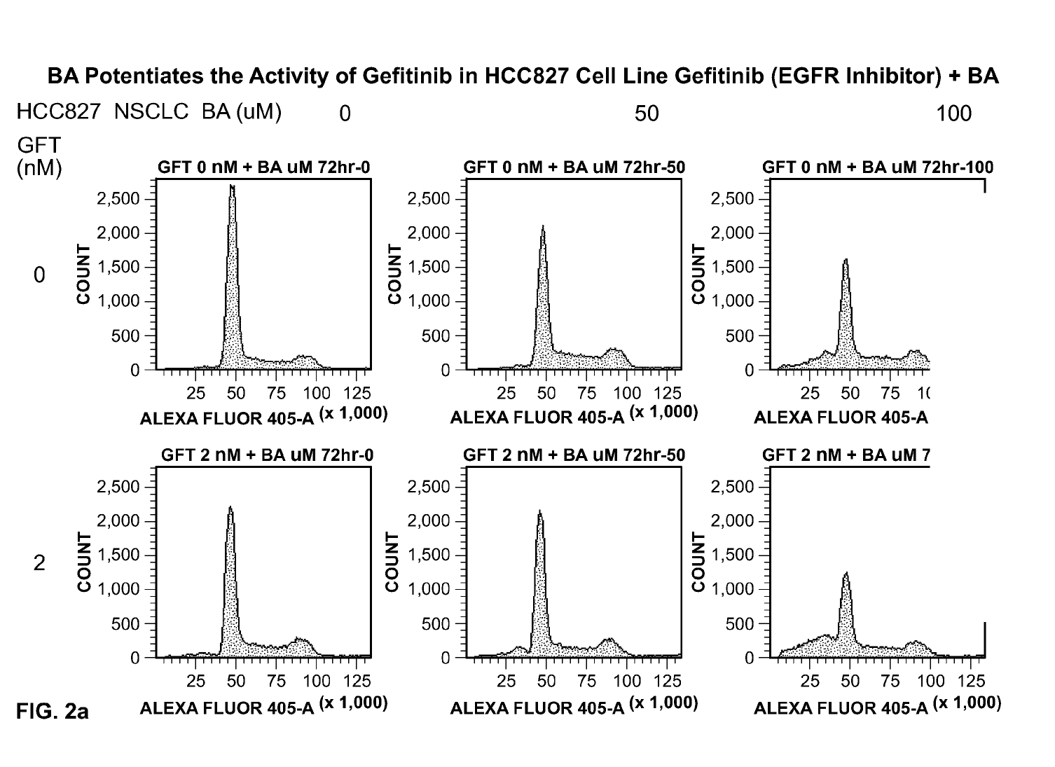

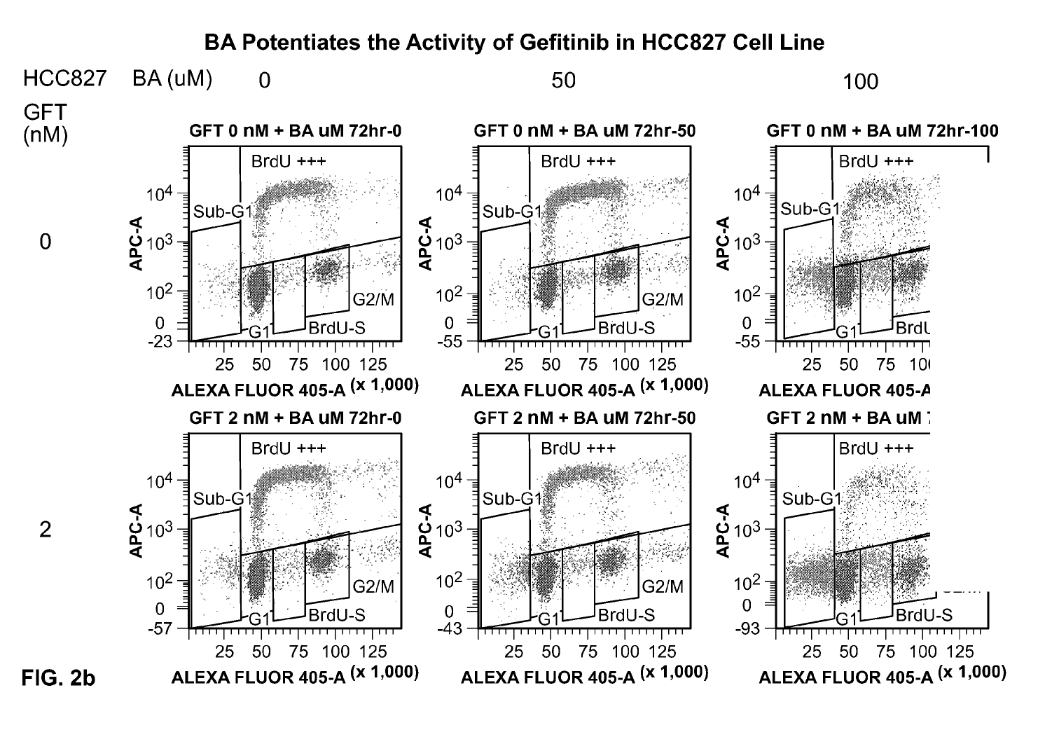

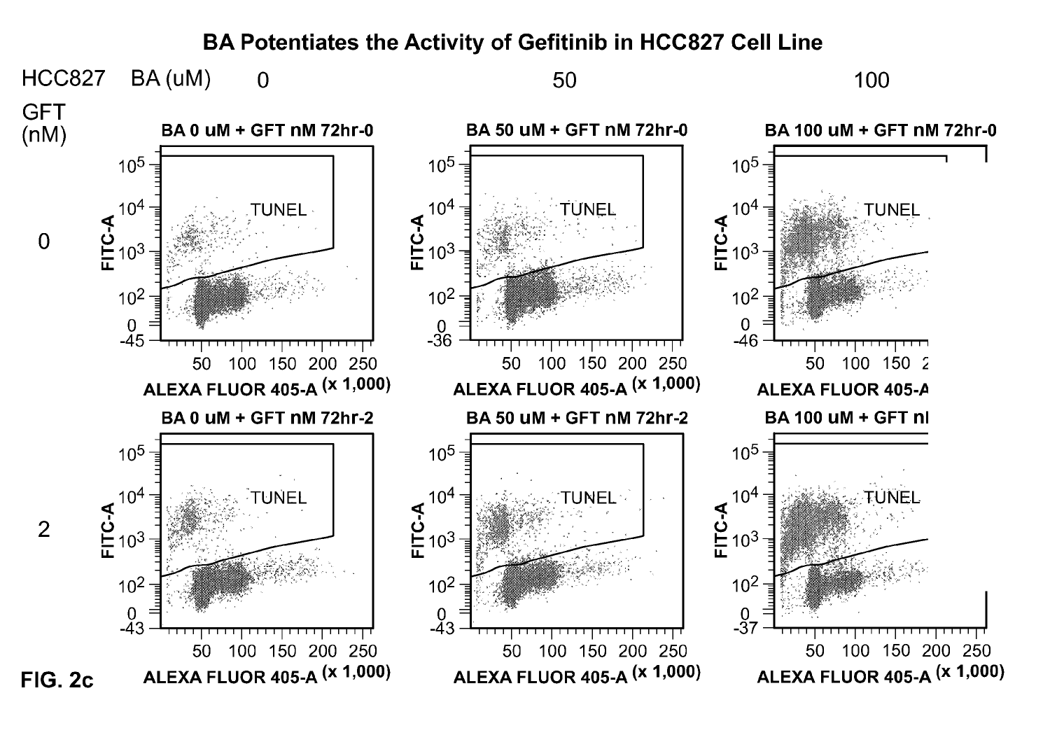

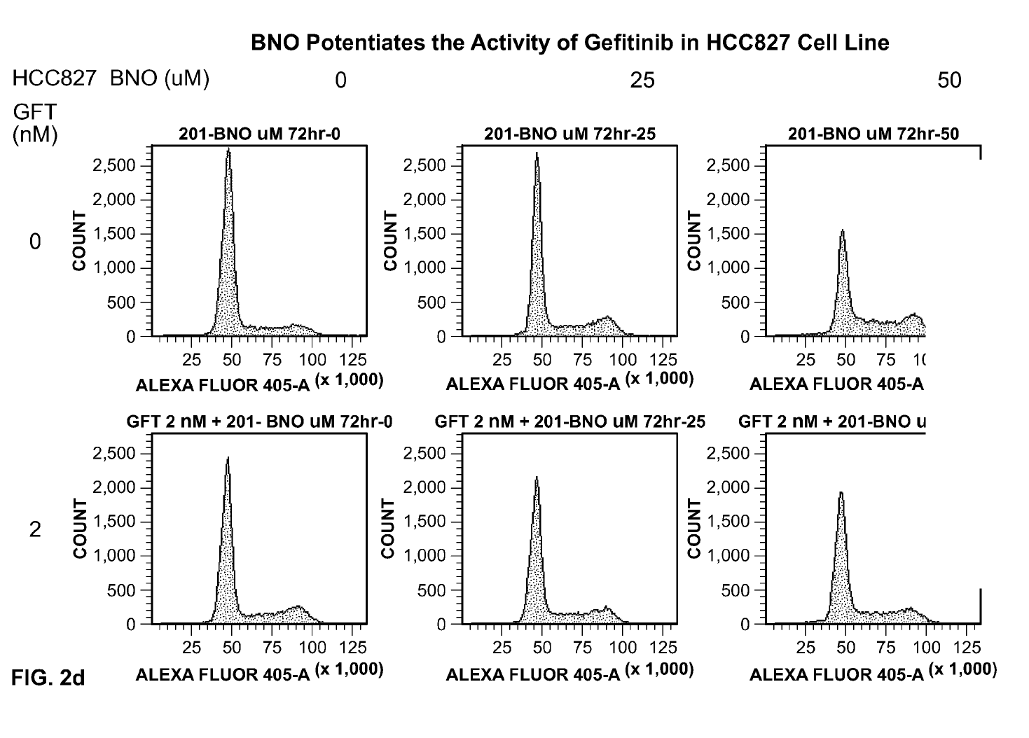

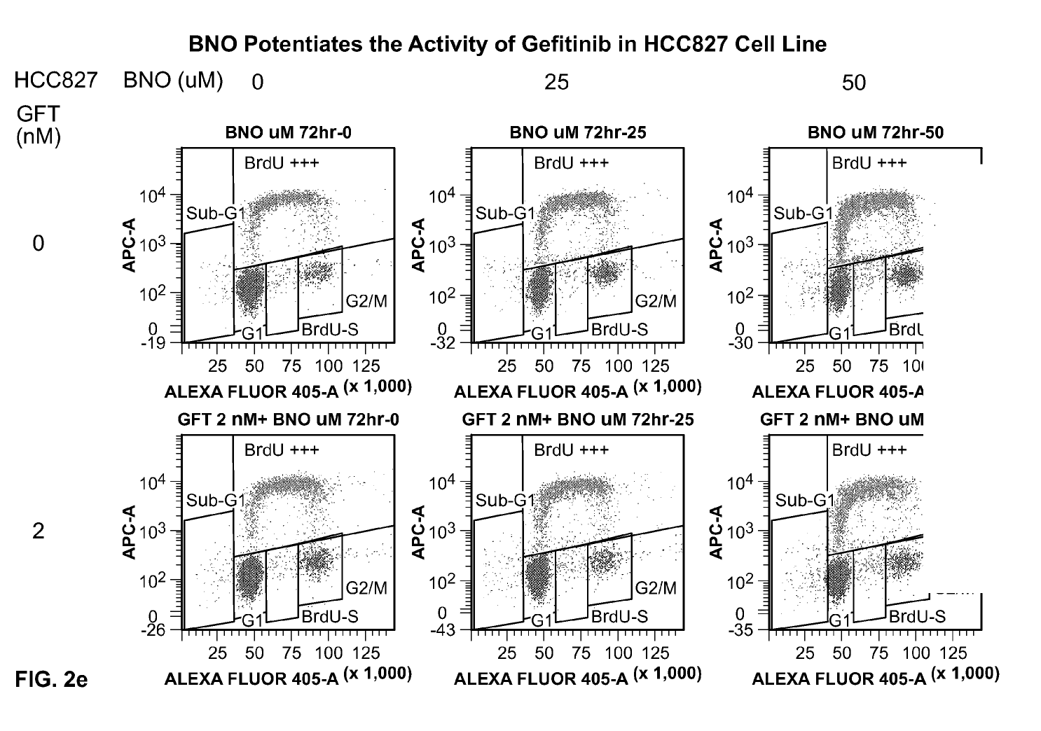

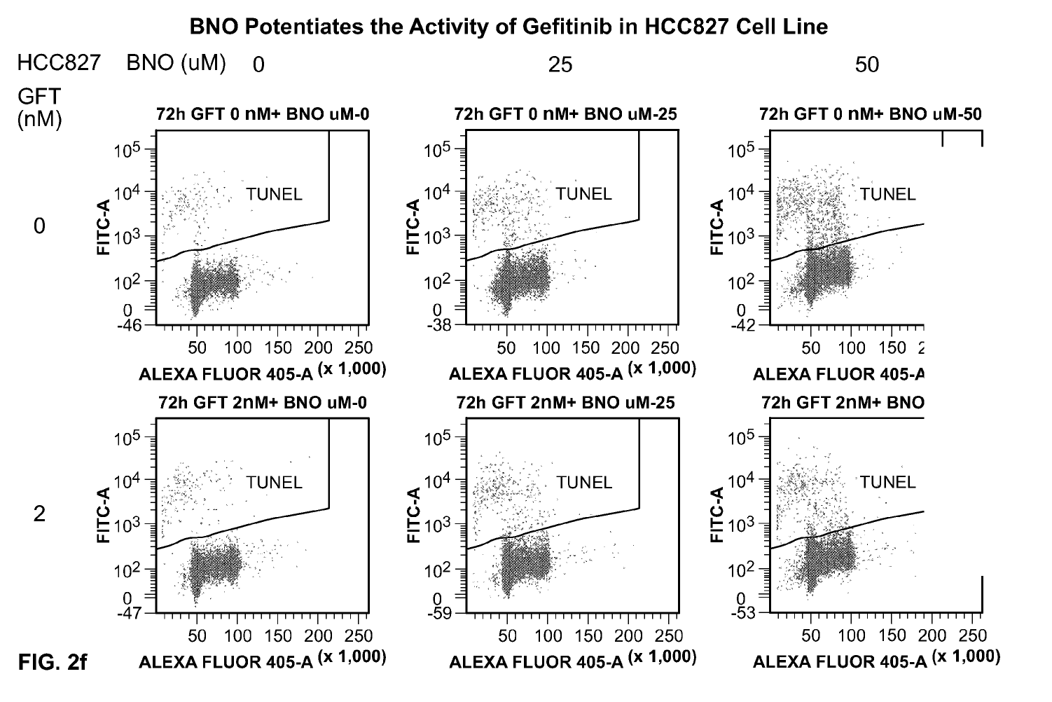

The active dose combinations were tested for their effects on cell survival, cell cycle distribution and BrdU incorporation by FACS analysis. The cell cycle distributions, as determined by DNA content and BrdU staining analyses for various stages of the cell cycle, are shown in FIG. 2 (a-f) and the quantification of the results presented in Table 3.

TABLE 3

| | | | | | | |
|---|---|---|---|---|---|---|
| | | | BrdU | | | TUNEL |
| Live Cell | Sub-G1 | G1 | (−) S | S | G2/M | (+) |

Gefitinib (EGFR inhibitor) + BA
HCC827 (NSCLC)

| | | | | | | | |
|---|---|---|---|---|---|---|---|
| GFT 0 nM + | | | | | | | |
| BA uM | | | | | | | |
| 0 | 100 | 1.94 | 65.85 | 1.63 | 24.26 | 6.32 | 3.2 |
| 50 | 65 | 2.36 | 46.26 | 1.95 | 40.62 | 8.82 | 5.7 |
| 100 | 25 | 15.79 | 43.76 | 8.05 | 19.92 | 12.49 | 32.8 |
| GFT 2 nM + | | | | | | | |
| BA uM | | | | | | | |
| 0 | 56 | 3.40 | 51.03 | 2.06 | 34.36 | 9.16 | 6.2 |
| 50 | 37 | 6.62 | 52.33 | 2.79 | 28.85 | 9.41 | 13.3 |
| 100 | 15 | 27.44 | 38.88 | 8.84 | 12.58 | 12.27 | 46.5 |

Gefitinib (EGFR inhibitor) + BNO
HCC827 (NSCLC)

| | | | | | | | |
|---|---|---|---|---|---|---|---|
| GFT 0 nM + | | | | | | | |
| BNO uM | | | | | | | |
| 0 | 100 | 0.90 | 72.82 | 1.10 | 20.96 | 4.21 | 1.3 |
| 25 | 76 | 1.10 | 61.10 | 1.30 | 29.30 | 7.20 | 2.9 |
| 50 | 40 | 2.10 | 38.74 | 3.50 | 44.24 | 11.41 | 7.7 |
| GFT 2 nM + | | | | | | | |
| BNO uM | | | | | | | |
| 0 | 57 | 1.01 | 64.69 | 1.21 | 25.75 | 7.34 | 1.5 |
| 25 | 34 | 1.41 | 61.45 | 1.61 | 28.71 | 6.83 | 3.3 |
| 50 | 30 | 1.91 | 56.14 | 2.62 | 31.09 | 8.25 | 4.5 |

### CONCLUSIONS

In non-small lung carcinoma cells, BA potentiates antitumor activity of gefitinib. FACS analysis and Tunel says have shown that BA has enhanced cell cycle arrest and induced apoptosis in gefitinib treated HCC827 cells.

### REFERENCES FOR EXAMPLES 1 AND 1a

1. Shi Y, Xiang R, Horváth C, Wilkins J A. Quantitative analysis of membrane proteins from breast cancer cell lines BT474 and MCF7 using multistep solid phase mass tagging and 2D LC/MS. J Proteome Res. 2005; (4):1427-33.
2. Sasaki K, Murakami T, Ogino T, Takahashi M, Kawasaki S. Flow cytometric estimation of cell cycle parameters using a monoclonal antibody to bromodeoxyuridine. Cytometry. 1986; (4):391-5.
3. Gorczyca W., Gong J., Darzynkiewicz Z. Detection of DNA strand breaks in individual apoptotic cells by the in situ terminal deoxynucleotidyl transferase and nick translation assays. Cancer Res. 1993; 3(8):1945-51.
4. Linn S C, Van't Veer L J, Clinical relevance of the triple-negative breast cancer concept: genetic basis and clinical utility of the concept. Eur J Cancer. 2009; 45 Suppl 1:11-26.
5. Chang B D, Broude E V, Fang J, Kalinichenko T V, Abdryashitov R, Poole J C, Roninson IB.p21Waf1/Cip1/Sdi1-induced growth arrest is associated with depletion of mitosis-control proteins and leads to abnormal mitosis and endoreduplication in recovering cells. Oncogene. 2000, 9(17):2165-70.
6. Chang B D, Watanabe K, Broude E V, Fang J, Poole J C, Kalinichenko T V, Roninson I B. Effects of p21Waf1/Cip1/Sdi1 on cellular gene expression: implications for carcinogenesis, senescence, and age-related diseases. Proc Natl Acad Sci USA. 2000; 97(8):4291-6.

### Example 2

### Measurement of Proliferation of the Lung Cell Line HCC827 Following Treatment with Ba, Alone and in Combination with Inhibitors of EGFR, FGFR, IGFR, HGFR, PDGFR, VEGFR, and NGFR

Lung epithelial adenocarcinoma cell line HCC827 was treated at multiple concentrations (100 μM and 50 μM) either alone or in combination with inhibitors of EGFR, FGFR, IGFR, HGFR, PDGFR, VEGFR, and NGFR. Each of the compounds was also tested on the cells as a single agent. The DMSO concentration was kept constant at 0.3% throughout all treatments. Following 72 hours of treatment, the effect of the treatments on the cell's rate of proliferation was measured using the CellTiter 96® Aqueous Cell Proliferation Assay which is a MTS-based assay similar to MTT. The assay was performed according supplier's instructions, see “CellTiter 96® Aqueous Non-Radioactive cell Proliferation Assay: Instructions for Use of Products G5421, G5430, G5440, G1111 and G1112,” Promega.com, Part#TB 169, 5/09, and references cited therein, all of which are incorporated by reference herein).

The CellTiter 96® AQueous Non-Radioactive Cell Proliferation Assay(a) is a colorimetric method for determining the number of viable cells in proliferation or chemosensitivity assays. The CellTiter 96® AQueous Assay is composed of solutions of a tetrazolium compound [3-(4,5-dimethylthiazol-2-yl)-5-(3-carboxymethoxyphenyl)-2-(4-sulfophenyl)-2H-tetrazolium, inner salt; MTS(a)] and an electron coupling reagent (phenazine methosulfate; PMS). MTS is bioreduced by cells into a formazan product that is soluble in tissue culture medium (Barltrop, J. A. et al. (1991) 5-(3-carboxymethoxyphenyl)-2-(4,5-dimethylthiazoly)-3-(4-sulfophenyl)tetrazolium, inner salt (MTS) and related analogs of 3-(4,5-dimethylthiazolyl)-2,5-diphenyltetrazolium bromide (MTT) reducing to purple water soluble formazans as cell-viability indicators. Bioorg. Med. Chem. Lett. 1, 611-4.). The absorbance of the formazan at 490 nm can be measured directly from 96-well assay plates without additional processing (Cory, A. H. et al. (1991) Use of an aqueous soluble tetrazolium/formazan assay for cell growth assays in culture. Cancer Comm. 3, 207-12; Riss, T. L. and Moravec, R. A. (1992) Comparison of MTT, XTT, and a novel tetrazolium compound MTS for in vitro proliferation and chemosensitivity assays. Mol. Biol. Cell (Suppl.) 3, 184a.). The conversion of MTS into aqueous, soluble formazan is accomplished by dehydrogenase enzymes found in metabolically active cells. The quantity of formazan product as measured by the amount of 490 nm absorbance is directly proportional to the number of living cells in culture.

Abbreviations: EGFR: Epidermal growth factor receptor; FGFR: Fibroblast growth factor receptor; IGFR: Insulin-like growth factor 1 receptor; HGFR: Hepatocyte growth factor receptor; PDGFR: Platelet-derived growth factor receptor; VEGFR: Vascular endothelial growth factor receptor; and NGFR: Nerve growth factor receptor.

### Materials:

Cell Line: HCC827 (CRL-2868, ATCC), an epithelial adenocarcinoma cell line

Gefitinib, EGFR inhibitor, Tocris Bioscience Cat#3000; MW=446.9

PD 173074, FGFR inhibitor (selective for FGFR1 and FGFR3), Tocris Bioscience Cat#3044; MW=523.67

Picropodophyllotoxin, IGFR, Tocris Bioscience Cat#2956

PHA 665752, HGFR inhibitor, Tocris Bioscience Cat#2693

DMPQ dihydrochloride, PDGFR inhibitor (selective inhibitor of human vascular β-type platelet derived growth factor receptor tyrosine kinase (β-type PDGFR tyrosine kinase)), Tocris Bioscience Cat#1222

SU4312, VEGFR inhibitor, Tocris Bioscience Cat#1459

K-252a, NGFR, inhibitor, LC Laboratories Cat# K2151

### Results and Conclusions:

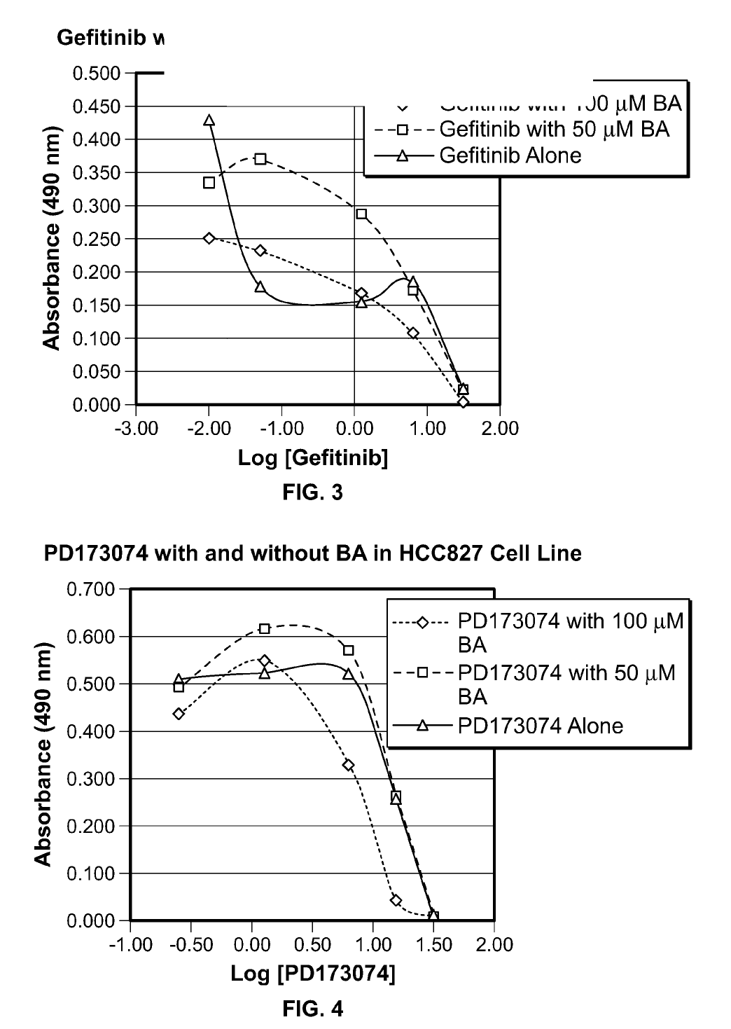

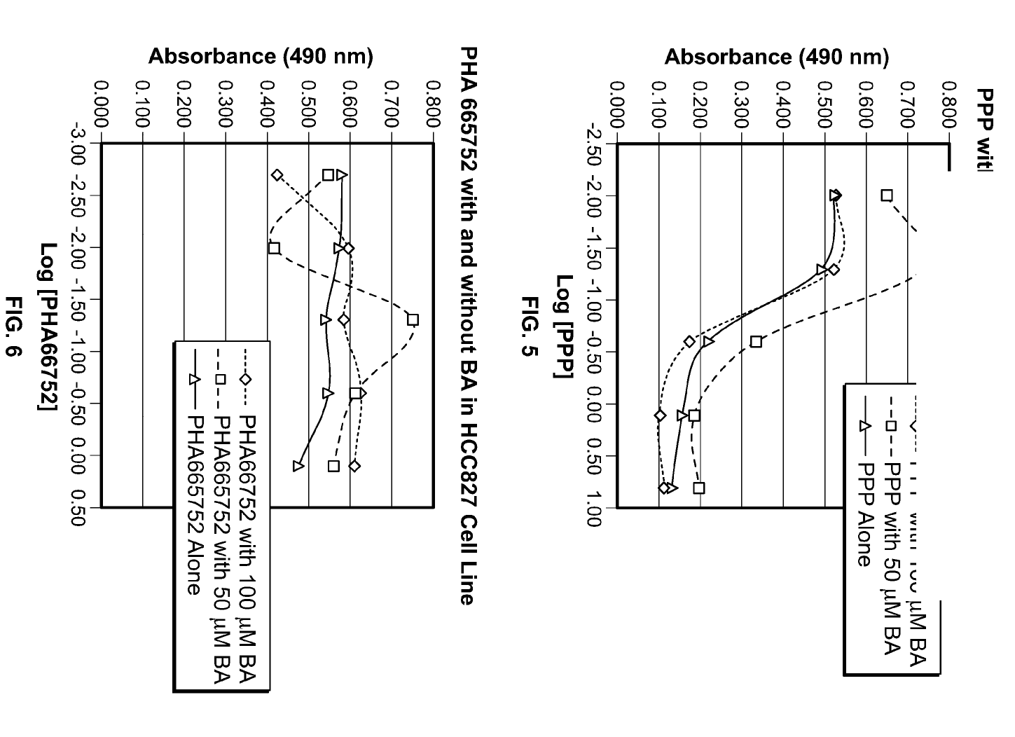

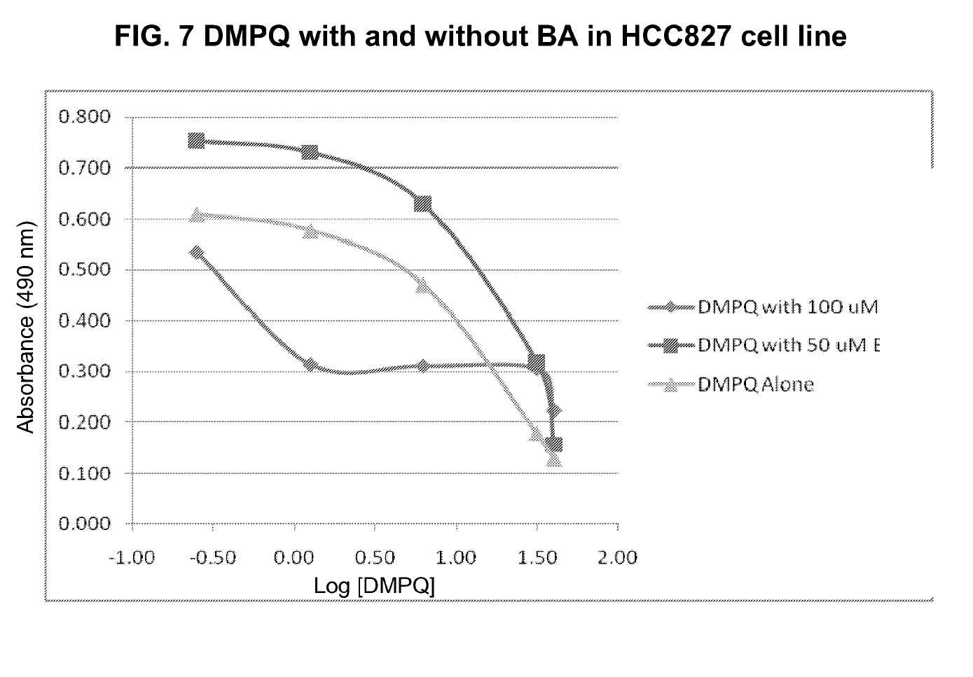

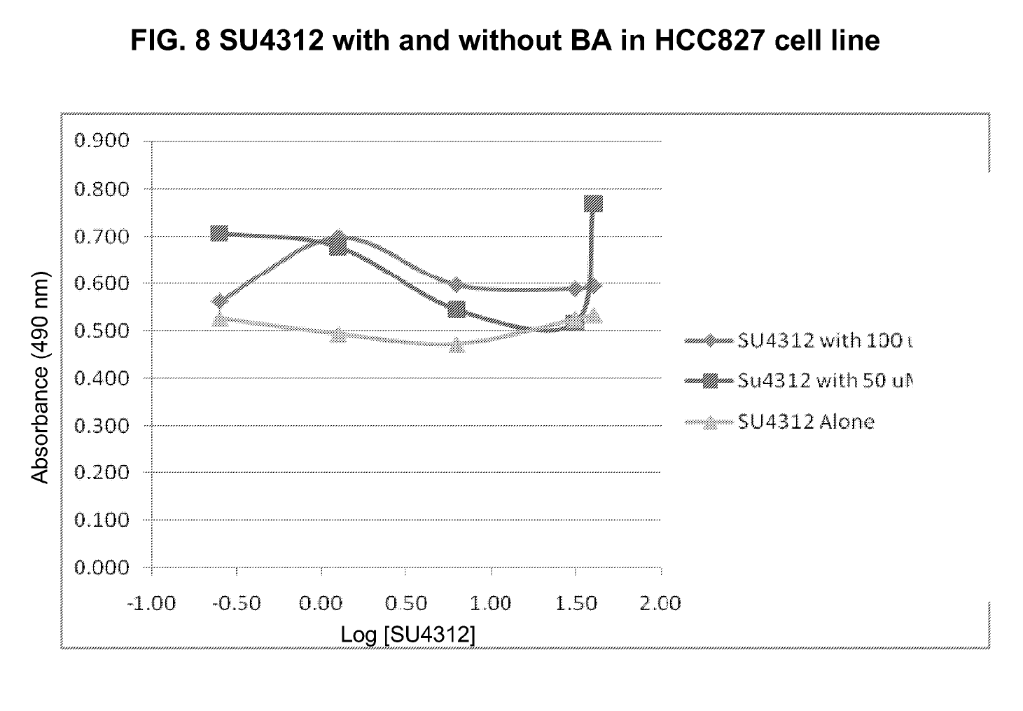

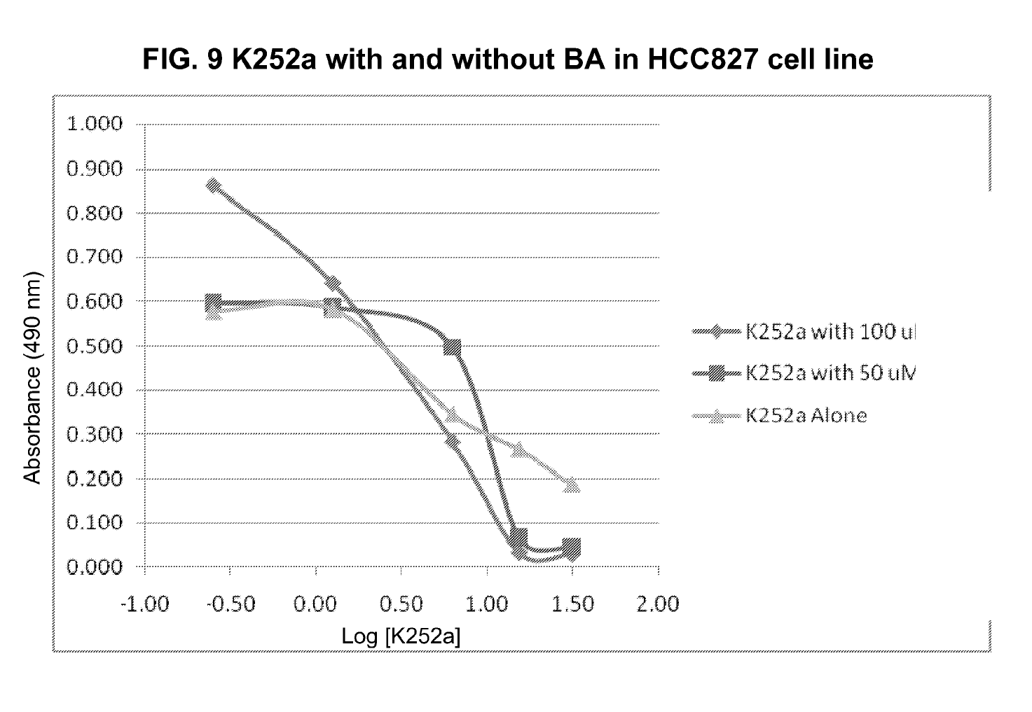

Data obtained from the CellTiter 96® Aqueous Cell Proliferation Assay are shown in FIGS. 3-9 for gefitinib (FIG. 3; an EGFR inhibitor), PD 173074 (FIG. 4, an FGFR inhibitor), picropodophyllotoxin (PPP) (FIG. 5; an IGF1R inhibitor, an IGF receptor subtype), PHA 665752 (FIG. 6, an HGFR inhibitor), DMPQ dihydrochloride (FIG. 7, a PDGFR inhibitor (specifically PDGFR-beta)), SU4312 (FIG. 8, a VEGFR inhibitor), and K252a (FIG. 9, an NGFR inhibitor) with and without BA.

Based on the results described herein, BA demonstrated potentiation of antiproliferative effects of growth factor receptors inhibitors, such as PD173074, PPP, DMPQ, K252a in non-small lung carcinoma HCC827 cells. The data suggest that these combinations can be potent tools to intercept growth factors stimulated tumor cell proliferation, motility and protection from apoptosis.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating lung cancer in a patient, comprising administering to the patient having lung cancer at least one nitrobenzamide compound in combination with at least one growth factor inhibitor wherein said nitrobenzamide compound is of Formula (Ia), or a metabolite thereof: 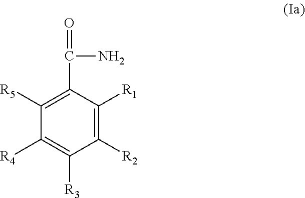
wherein R₁, R₂, R₃, R₄, and R₅ are, independently selected from the group consisting of hydrogen, hydroxy, amino, nitro, nitroso, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents is always nitro, and at least one substituent positioned adjacent to a nitro is always iodo, or a pharmaceutically acceptable salt thereof, and,
wherein the growth factor inhibitor is selected from the group consisting of AEE788, GW-974, BIBW 2992, catumaxomab, EGF vaccine, icotinib, leflunomide, necitumumab, neratinib, pertuzumab, PF-299804, zalutumumab, CNTF, tanezumab, dalotuzumab, AMG-479, rilotumumab, lanreotide, OSI 906, pasireotide, PF-2341066, MetMab, XL-184, aflibercept, apatinib, BIBF-1120, PAM-1, XL-999, brivanib, fluocinolone, midostaurin, motesanib, OTS-102, OSI-632, vatalanib, pazopanib, BMS-690514, ramucirumab, ridoforolimus, tivozanib, alacizumab pegol, PD173074, PHA 665752, DMQ, SU4312, K252a, XL-647, VEGF-Trap-Eye, pirfenidone, masitinib, and nilotinib.

2. The method of claim 1, wherein at least one therapeutic effect is obtained, said at least one therapeutic effect being reduction in size of a lung tumor, reduction in metastasis, complete remission, partial remission, stable disease, or a pathologic complete response.

3. The method of claim 1, wherein an improvement of clinical benefit rate (CBR=CR (complete remission)+PR (partial remission)+SD (stable disease)≧6 months) is obtained as compared to treatment with the growth factor inhibitor administered without the nitrobenzamide compound.

4. The method of claim 3, wherein the improvement of clinical benefit rate is about 60% or higher.

5. The method of claim 1, wherein the nitrobenzamide compound is 4-iodo-3-nitrobenzamide, or pharmaceutically acceptable salt thereof.

6. The method of claim 1, wherein the growth factor is an epidermal growth factor receptor (EGFR) inhibitor selected from the group consisting of BIBW 2992, catumaxomab, XL-647, EGF vaccine, icotinib, leflunomide, necitumumab, neratinib, GW-974, PF-299804, and zalutumumab.

7. The method of claim 1, wherein the growth factor inhibitor is a nerve growth factor receptor (NGFR) inhibitor selected from the group consisting of CNTF, K252a, and tanezumab.

8. The method of claim 1, wherein the growth factor inhibitor is an insulin-like growth factor I (IGF1) receptor inhibitor selected from the group consisting of dalotuzumab, AMG-479, rilotumumab, lanreotide, OSI 906, and pasireotide.

9. The method of claim 1, wherein the growth factor inhibitor is a hepatocyte growth factor receptor (HGFR) inhibitor selected from the group consisting of PF-2341066, MetMab, PHA 665752, and XL-184.

10. The method of claim 1, wherein the growth factor inhibitor is a vascular endothelial growth factor receptor (VEGFR) inhibitor selected from the group consisting of aflibercept, apatinib, BIBF-1120, brivani, fluocinolone, midostaurin, motesanib, OTS-102, OSI-632, vatalanib, pazopanib, BMS-690514, ramucirumab, ridoforolimus, tivozanib, XL-647, VEGF-Trap-Eye, alacizumab pegol, SU4312, and XL-184.

11. The method of claim 1, wherein the growth factor inhibitor is a fibroblast growth factor receptor (FGFR) inhibitor selected from the group consisting of BIBF-1120, brivanib, PAM-1, pirfenidone, PD 173074, and masitib.

12. The method of claim 1, wherein the growth factor inhibitor is a platelet derived growth factor receptor (PDGFR) inhibitor selected from the group consisting of BIBF-1120, leflunomide, masitinib, motesanib, nilotinib, pazopanib, pirfenidone, DMPQ, SU4312, and tivozanib.

13. The method of claim 1, wherein the growth factor inhibitor is a platelet derived growth factor receptor (PDGFR) inhibitor and is pazopanib.

14. The method of claim 1, wherein the growth factor inhibitor is AEE788.

15. The method of claim 1, further comprising surgery, radiation therapy, chemotherapy, gene therapy, DNA therapy, viral therapy, DNA therapy, adjuvant therapy, neoadjuvant therapy, RNA therapy, immunotherapy, nanotherapy or a combination thereof.

16. The method of claim 1, wherein the lung cancer is a metastatic lung cancer.

17. The method of claim 1, wherein the lung cancer is at stage I, stage II, or stage III.

18. The method of claim 1, wherein the lung cancer is a non-small cell lung carcinoma (NSCLC).

19. The method of claim 18, wherein the non-small cell lung carcinoma is a squamous cell carcinoma, adenocarcinoma, or large cell carcinoma.

20. The method of claim 1, wherein the lung cancer is a small cell lung carcinoma (SCLC).

21. The method of claim 1, wherein the lung cancer is deficient in homologous recombination DNA repair.

22. The method of claim 1, wherein the growth factor inhibitor is administered as a parenteral injection or infusion.

23. The method of claim 1, wherein the nitrobenzamide compound is 4-iodo-3-nitrobenzamide, which is administered orally, or as a parenteral injection or infusion, or by inhalation.

24. The method of claim 1, further comprising administering to the patient one or more of the group consisting of a cyclodextrin, a surfactant, and a co-solvent in combination with the nitrobenzamide compound.

25. The method of claim 24, wherein the cyclodextrin is selected from the group consisting of hydroxypropyl-β-cyclodextrin, hydroxypropyl-γ-cyclodextrin, and sulfobutyl ether-β-cyclodextrin, or a combination thereof.

26. The method of claim 18, wherein at least one therapeutic effect is obtained, said at least one therapeutic effect being reduction in size of a non-small cell lung tumor, reduction in metastasis, complete remission, partial remission, stable disease, or a pathologic complete response.

27. The method of claim 18, wherein an improvement of clinical benefit rate (CBR=CR (complete remission)+PR (partial remission)+SD (stable disease)≧6 months) is obtained as compared to treatment with the growth factor inhibitor administered without the nitrobenzamide compound.

28. The method of claim 27, wherein the improvement of clinical benefit rate is about 60% or higher.

29. The method of claim 18, wherein the nitrobenzamide compound is 4-iodo-3-nitrobenzamide, or a pharmaceutical salt thereof, or a metabolite thereof.

30. The method of claim 18, wherein the growth factor is an epidermal growth factor receptor (EGFR) inhibitor selected from the group consisting of BIBW 2992, catumaxomab, EGF vaccine, icotinib, leflunomide, necitumumab, neratinib, and zalutumumab.

31. The method of claim 18, wherein the growth factor inhibitor is pazopanib.

32. The method of claim 18, wherein the growth factor inhibitor is AEE788.

33. The method of claim 18, further comprising surgery, radiation therapy, chemotherapy, gene therapy, DNA therapy, viral therapy, DNA therapy, adjuvant therapy, neoadjuvant therapy, RNA therapy, immunotherapy, nanotherapy or a combination thereof.

34. The method of claim 18, wherein the non-small cell lung cancer is a metastatic non-small cell lung cancer.

35. The method of claim 1, wherein the non-small cell lung carcinoma is a squamous cell carcinoma, adenocarcinoma, or large cell carcinoma.

36. The method of claim 18, wherein the non-small cell lung cancer is deficient in homologous recombination DNA repair.

37. The method of claim 18, wherein the growth factor inhibitor is administered as a parenteral injection or infusion.

38. The method of claim 18, wherein the nitrobenzamide compound is 4-iodo-3-nitrobenzamide, or pharmaceutically acceptable salt thereof.

39. The method of claim 29, wherein the 4-iodo-3-nitrobenzamide, or pharmaceutically acceptable salt thereof, is administered orally, or as a parenteral injection or infusion, or by inhalation.
